(19) United States
(12) Patent Application Publication
(10) Pub. No.: US 2023/0310453 A1
(43) Pub. Date: Oct. 5, 2023

(54) METHODS FOR EXTRACTING NEUTROPHIL SERINE PROTEASES AND TREATING DIPEPTIDYL PEPTIDASE 1-MEDIATED CONDITIONS

(71) Applicant: INSMED, INC., Bridgewater, NJ (US)

(72) Inventors: Jessica BASSO, Bridgewater, NJ (US); Jimin ZHANG, Bridgewater, NJ (US); David C. CIPOLLA, Bridgewater, NJ (US)

(21) Appl. No.: 18/017,261

(22) PCT Filed: Jul. 19, 2021
(86) PCT No.: PCT/US2021/042199

§ 371 Date: Jan. 20, 2023

Related U.S. Application Data
(60) Provisional application No. 63/215,599, filed on Jun. 28, 2021.
(60) Provisional application No. 63/053,939, filed on Jul. 20, 2020.

(51) Int. Cl. A61K 31/553; A61K 9/00; A61P 11/00; C12N 9/64; G01N 33/573

(52) U.S. Cl. CPC A61K 31/553; A61K 9/0053; A61P 11/00; C12N 9/6424; C12Y 304/21076; G01N 33/573; G01N 2333/96433

(57) ABSTRACT

Methods are provided for extracting one or more neutrophil serine proteases (NSPs), e.g., neutrophil elastase (NE), proteinase 3 (PR3), cathepsin G (CatG), neutrophil serine protease 4 (NSP4), or a combination thereof, from a sample comprising white blood cells (WBCs) obtained from a subject. The extraction methods feature the use of nonionic surfactants and two or more cycles of repeated lysis of WBCs and their residuals. Also provided are methods for treating dipeptidyl peptidase 1 (DPP1)-mediated conditions in a patient with compositions comprising certain N-(1-cyano-2-phenylethyl)-1,4-oxazepane-2-carboxamide compounds of formula (I), including pharmaceutically acceptable salts thereof,

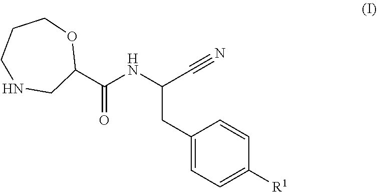

that reversibly inhibit (DPP1) activity. The treatment methods provided herein use the concentration of active NSPs extracted from a patient's WBC sample as a biomarker to guide the selection of, or adjustment to, an effective dosage of the compounds of formula (I).

### CROSS REFERENCE TO RELATED APPLICATIONS

This application is a National Stage of International Patent Application Number PCT/US2021/042199, filed Jul. 19, 2021, which claims priority from U.S. Provisional Application No. 63/053,939, filed Jul. 20, 2020, and U.S. Provisional Application No. 63/215,599, filed Jun. 28, 2021, the disclosure of each of which is incorporated by reference herein in its entirety.

### BACKGROUND OF THE INVENTION

Neutrophil serine proteases (NSPs) reside inside the azurophilic granules of neutrophils. With broad substrate specificity, NSPs are important for the functioning of neutrophils, playing key roles in immune protection against bacterial infections and in the regulation of inflammatory conditions. Known NSPs include neutrophil elastase (NE), proteinase 3 (PR3), cathepsin G (CatG), and neutrophil serine protease 4 (NSP4). The classic NSPs, i.e., NE, PR3, and CatG, are synthesized during the pro-myelocytic stage of neutrophil differentiation as inactive zymogens, which are activated by the cysteine protease dipeptidyl peptidase 1 (DPP1; also known as cathepsin C) via proteolytic processing at the amino terminus. Discovered recently, NSP4 has 39% identity with NE and PR3 and exhibits restricted expression in neutrophilic granulocytes and bone-marrow precursor cells. Like NE, PR3, and CatG, NSP4 is converted into an active protease by DPP1 via proteolytic processing at the amino terminus. See Pham et al., Nature Reviews Immunology, 6:541-550 (2006); Perera et al, PNAS, 109:6229-6234 (2012), each of which is incorporated herein by reference in its entirety for all purposes.

Because NSPs are implicated in various disease pathways, the ability to effectively measure the concentration of active NSPs from blood samples could provide insight into disease progression and serve as a biomarker. The present invention addresses this and other needs.

### SUMMARY OF THE INVENTION

In one aspect, the present application relates to a method of extracting one or more neutrophil serine proteases (NSPs) from a sample comprising white blood cells (WBCs) obtained from a subject. The method includes: contacting the sample with a first aqueous medium comprising at least 0.01% (v/v) of a first nonionic surfactant to obtain a first cell lysate comprising a first NSP extract, and a first WBC residual, wherein the first NSP extract comprises the one or more NSPs, separating the first cell lysate from the first WBC residual, to provide a first separated cell lysate comprising the first NSP extract, contacting the first WBC residual with a second aqueous medium comprising at least 0.01% (v/v) of a second nonionic surfactant to obtain a second cell lysate comprising a second NSP extract, and a second WBC residual, wherein the second NSP extract comprises the one or more NSPs, and separating the second cell lysate from the second WBC residual to provide a second separated cell lysate comprising the second NSP extract.

In some embodiments of the method, additional repeated lysis steps are carried out. In one embodiment, the method further comprises contacting the second WBC residual with a third aqueous medium comprising at least 0.01% (v/v) of a third nonionic surfactant to obtain a third cell lysate comprising a third NSP extract, and a third WBC residual, wherein the third NSP extract comprises the one or more NSPs, and separating the third cell lysate from the third WBC residual to provide a third separated cell lysate comprising the third NSP extract. In a further embodiment, the method comprises contacting the third WBC residual with a fourth aqueous medium comprising at least 0.01% (v/v) of a fourth nonionic surfactant to obtain a fourth cell lysate comprising a fourth NSP extract, and a fourth WBC residual, wherein the fourth NSP extract comprises the one or more NSPs, and separating the fourth cell lysate from the fourth WBC residual to provide a fourth separated cell lysate comprising the fourth NSP extract. In even a further embodiment, the method comprises contacting the fourth WBC residual with a fifth aqueous medium comprising at least 0.01% (v/v) of a fifth nonionic surfactant to obtain a fifth cell lysate comprising a fifth NSP extract, and a fifth WBC residual, wherein the fifth NSP extract comprises the one or more NSPs, and separating the fifth cell lysate from the fifth WBC residual to provide a fifth separated cell lysate comprising the fifth NSP extract. In still a further embodiment, the method comprises contacting the fifth WBC residual with a sixth aqueous medium comprising at least 0.01% (v/v) of a sixth nonionic surfactant to obtain a sixth cell lysate comprising a sixth NSP extract, and a sixth WBC residual, wherein the sixth NSP extract comprises the one or more NSPs, and separating the sixth cell lysate from the sixth WBC residual to provide a sixth separated cell lysate comprising the sixth NSP extract.

In one embodiment of the method, the nonionic surfactant present in any one of the aqueous media is a nonionic polyoxyethylene surfactant, e.g., octylphenoxypolyethoxyethianol, 2-[4-(2,4,4-trimethylpentan-2-yl)phenoxy]ethanol, polyoxyethylene (9) nonylphenylether (branched), or polyoxyethylene (20) sorbitan monolaurate. In a further embodiment, the surfactant present in any one of the aqueous media is octylphenoxypolyethoxyethanol.

In one embodiment of the method where two or more repeated lysis steps are carried out, the nonionic surfactant present in an aqueous medium used for each lysis step is the same. In another embodiment, the nonionic surfactants present in at least two of the aqueous media used are different. Regardless of the identity of the surfactants present in the aqueous media, in one embodiment, the concentration of the nonionic surfactant present in an aqueous medium used for each lysis step is the same. In another embodiment, the concentrations of the nonionic surfactants in at least two of the aqueous media used are different.

In one embodiment of the method, all of the aqueous media used (e.g., all of the first aqueous medium, the second aqueous medium, the third aqueous medium, etc., depending on the number of repeated lysis steps performed) are the same aqueous medium. In another embodiment, at least two of the aqueous media are different.

In one embodiment of the method, the first, second, third, fourth, fifth, or sixth aqueous medium, or a combination thereof comprises at least 0.02% (v/v) of the respective first, second, third, fourth, fifth, or sixth nonionic surfactant. In another embodiment, the first, second, third, fourth, fifth, or sixth aqueous medium, or a combination thereof comprises at least 0.05% (v/v) of the respective first, second, third, fourth, fifth, or sixth nonionic surfactant. In another embodiment, the first, second, third, fourth, fifth, or sixth aqueous medium, or a combination thereof comprises from about 0.02% (v/v) to about 1.5% (v/v) of the respective first, second, third, fourth, fifth, or sixth nonionic surfactant. In another embodiment, the first, second, third, fourth, fifth, or sixth aqueous medium, or a combination thereof comprises from about 0.03% (v/v) to about 1% (v/v) of the respective first, second, third, fourth, fifth, or sixth nonionic surfactant. In another embodiment, the first, second, third, fourth, fifth, or sixth aqueous medium, or a combination thereof comprises from about 0.04% (v/v) to about 0.8% (v/v) of the respective first, second, third, fourth, fifth, or sixth nonionic surfactant. In another embodiment, the first, second, third, fourth, fifth, or sixth aqueous medium, or a combination thereof comprises from about 0.05% (v/v) to about 0.6% (v/v) of the respective first, second, third, fourth, fifth, or sixth nonionic surfactant. In another embodiment, the first, second, third, fourth, fifth, or sixth aqueous medium, or a combination thereof comprises about 0.05% (v/v) of the respective first, second, third, fourth, fifth, or sixth nonionic surfactant. In a further embodiment, the respective first, second, third, fourth, fifth, or sixth nonionic surfactant, or a combination thereof, is a nonionic polyoxyethylene surfactant. In a further embodiment, the nonionic polyoxyethylene surfactant is octylphenoxypolyethoxyethanol. In a further embodiment, the first, second, third, fourth, fifth, or sixth aqueous medium, or a combination thereof comprises about 0.05% (v/v) of octylphenoxypolyethoxyethanol, about 0.75 M NaCl, and about 50 mM HEPES.

In one embodiment of the method, the sample comprising WBCs is contacted with the first aqueous medium at a temperature of from about 0° C. to about 10° C. In another embodiment, a WBC residual (e.g., a first, second, third, fourth, or fifth WBC residual) is contacted with a corresponding aqueous medium at a temperature of from about 0° C. to about 10° C.

In one embodiment of the method, contacting the sample comprising WBCs with the first aqueous medium includes mixing the sample with the first aqueous medium. In a further embodiment, mixing the sample with the first aqueous medium includes agitating the sample with the first aqueous medium. In another embodiment, contacting a WBC residual (e.g., a first, second, third, fourth, or fifth WBC residual) with a corresponding aqueous medium includes mixing the WBC residual with the corresponding aqueous medium. In a further embodiment, mixing the WBC residual with the corresponding aqueous medium includes agitating the WBC residual with the corresponding aqueous medium. The agitating mentioned above can be carried out by pipetting, vortexing, shaking, stirring, or using a paddle, such as a United States Pharmacopeia (USP) apparatus 2.

In one embodiment of the method, contacting the sample with a first aqueous medium comprises adding an aqueous wash solution to the sample to form a mixture of the aqueous wash solution and the sample, centrifuging the mixture of the aqueous wash solution and the sample to provide a supernatant (i.e., wash fraction) and a pellet comprising the WBCs, collecting the supernatant, and contacting the pellet with the first aqueous medium. In one embodiment, the aqueous wash solution is a phosphate buffered saline solution. In another embodiment, the aqueous wash solution is a saline solution comprising about 0.9% NaCl. In another embodiment, the aqueous wash solution comprises a Tris-based alkaline buffer and NaCl. In a further embodiment, the aqueous wash solution comprises about 100 mM Tris and about 100 mM NaCl with a pH of about 7.5. In a further embodiment, the supernatant (i.e., wash fraction) comprises the one or more NSPs, and the method further comprises measuring a concentration of an active form of the one or more NSPs of the supernatant.

In one embodiment of the method, the method further comprises measuring a concentration of an active form of the one or more NSPs of individual separated cell lysates (e.g., a first, second, third, fourth, fifth, or sixth separated cell lysate). Alternatively or additionally, the method comprises combining two or more separated cell lysates to provide a pooled cell lysate comprising a pooled NSP extract that contains the one or more NSPs, optionally followed by measuring a concentration of an active form of the one or more NSPs of the pooled cell lysate comprising the pooled NSP extract. In an exemplary embodiment, the method comprises combining all of the separated cell lysates to provide a single pooled cell lysate. In a further embodiment, the concentration of an active form of the one or more NSPs of the single pooled cell lysate is measured.

In one embodiment of the method, the one or more NSPs comprise neutrophil elastase (NE), proteinase 3 (PR3), cathepsin G (CatG), neutrophil serine protease 4 (NSP4), or a combination thereof. In another embodiment, the one or more NSPs comprise NE. In another embodiment, the one or more NSPs comprise PR3. In another embodiment, the one or more NSPs comprise CatG. In another embodiment, the one or more NSPs comprise NSP4.

In one embodiment of the method, the subject is a human subject.

In another aspect, the present disclosure relates to a method of treating a DPP1-mediated condition in a patient in need thereof. The method includes:
(a) measuring a baseline concentration of an active form of one or more NSPs extracted from a first sample comprising white blood cells obtained from the patient,
(b) orally administering to the patient daily for a first administration period of about 2 weeks to about 16 weeks, a pharmaceutical composition comprising a first daily dosage of about 10 mg to about 40 mg of a compound of formula (I), or a pharmaceutically acceptable salt thereof,

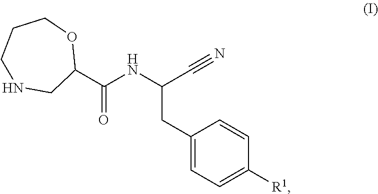

wherein,
R₁ is

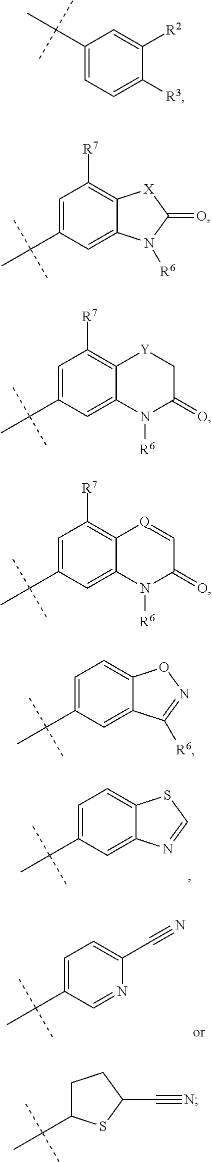

R² is hydrogen, F, Cl, Br, OSO₂C₁₋₃alkyl, or C₁₋₃alkyl;
R³ is hydrogen, F, Cl, Br, CN, CF₃, SO₂C₁₋₃alkyl, CONH₂ or SO₂NR⁴R⁵,
wherein R⁴ and R⁵ together with the nitrogen atom to which they are attached form an azetidine, pyrrolidine or piperidine ring;
X is O, S or CF₂;
Y is O or S;
Q is CH or N;
R⁶ is C₁₋₃alkyl, wherein the C₁₋₃alkyl is optionally substituted by 1, 2 or 3 F and optionally by one substituent selected from OH, OC₁₋₃alkyl, N(C₁₋₃alkyl)₂, cyclopropyl, or tetrahydropyran; and
R⁷ is hydrogen, F, Cl or CH₃;
(c) measuring a concentration of the active form of the one or more NSPs extracted from a second sample comprising white blood cells, wherein the second sample is obtained from the patient during the first administration period, or about one week or less subsequent to the first administration period,
(d) comparing the concentration from the second sample with the baseline concentration from the first sample; and
if the concentration from the second sample is reduced by about 10% or more as compared to the baseline concentration from the first sample, then orally administering to the patient daily for a second administration period the same daily dosage as the first daily dosage of the compound of formula (I), or a pharmaceutically acceptable salt thereof, or
if the concentration from the second sample is not reduced by about 10% or more as compared to the baseline concentration from the first sample, then orally administering to the patient daily for a second administration period a second daily dosage of the compound of formula (I), or a pharmaceutically acceptable salt thereof, wherein the second daily dosage is about 1.5 times to about 7 times the first daily dosage.

In some embodiments of the method, the first daily dosage of the compound of formula (I), or a pharmaceutically acceptable salt thereof is about 10 mg to about 25 mg, about 10 mg to about 15 mg, about 10 mg to about 12 mg, about 16 mg to about 25 mg, or about 20 mg to about 25 mg.

In some embodiments of the method, the second daily dosage is about 1.5 times to about 6 times, about 1.5 times to about 5 times, about 1.5 times to about 4 times, about 1.5 times to about 3 times, or about 1.5 times to about 2 times the first daily dosage.

In some embodiments of the method, the first administration period is about 2 weeks to about 12 weeks, about 2 weeks to about 8 weeks, about 3 weeks to about 6 weeks, about 3 weeks to about 5 weeks, e.g., about 3, 4, 5, 6, 7, 8, 9, 10, 11, or 12 weeks.

In some embodiments of the method, the second sample is obtained from the patient during the first administration period. For example, the second sample may be obtained from the patient at the end of the first administration period, or about 1, 2, 3, 4, 5, 6, or 7 days before the end of the first administration period. In one embodiment, the first administration period is about 4 weeks, and the second sample is obtained from the patient at about 4 weeks during the first administration period.

In some embodiments of the method, the second sample is obtained from the patient about one week subsequent to the first administration period. In other embodiments, the second sample is obtained from the patient about 1, 2, 3, 4, 5, 6, or 7 days subsequent to the first administration period.

In one embodiment of the method, the one or more NSPs comprise NE. In a further embodiment, if the concentration of the active form of NE from the second sample is reduced by about 19% or more as compared to the baseline concentration of the active form of NE from the first sample, then orally administering daily for the second administration period the same daily dosage as the first daily dosage of the compound of formula (I), or a pharmaceutically acceptable salt thereof, or if the concentration of the active form of NE from the second sample is not reduced by about 19% or more as compared to the baseline concentration of the active form of NE from the first sample, then orally administering daily for the second administration period the second daily dosage of the compound of formula (I), or a pharmaceutically acceptable salt thereof.

In some embodiments of the method, the second administration period is at least 1 month, e.g., from about 1 month to about 24 months, from about 1 month to about 12 months, from about 5 months to about 24 months, from about 5 months to about 18 months, or from about 5 months to about 15 months, from about 3 months to about 6 months, from about 6 months to about 12 months, from about 12 months to about 18 months, or from about 12 months to about 24 months.

In one embodiment of the method, the compound of formula (I) is (2S)—N-{(1S)-1-cyano-2-[4-(3-methyl-2-oxo-2,3-dihydro-1,3-benzoxazol-5-yl)phenyl]ethyl}-1,4-oxazepane-2-carboxamide, referred to herein by its international nonproprietary name (INN), brensocatib (and formerly known as INS1007 and AZD7986),

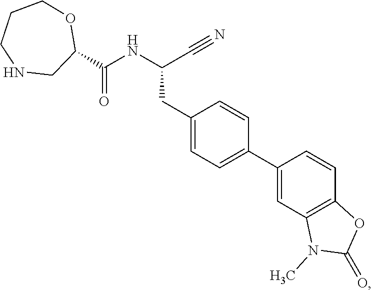 [SMILES: Cn1c(=O)oc2ccc(-c3ccc(C[C@@H](C#N)NC(=O)[C@@H]4CNCCCO4)cc3)cc21]

or a pharmaceutically acceptable salt thereof.

In one embodiment of the method, the DPP1-mediated condition is an obstructive disease of the airways. In one embodiment, the obstructive disease of the airways is bronchiectasis. In a further embodiment, the bronchiectasis is non-cystic fibrosis bronchiectasis. In another embodiment, the obstructive disease of the airways is cystic fibrosis. In another embodiment, the obstructive disease of the airways is alpha-1 antitrypsin deficiency.

In one embodiment of the method, the DPP1-mediated condition is cancer, e.g., breast cancer, bladder cancer, lung cancer, brain cancer, ovarian cancer, pancreatic cancer, colorectal cancer, prostate cancer, liver cancer, hepatocellular carcinoma, kidney cancer, stomach cancer, skin cancer, fibroid cancer, lymphoma, virus-induced cancer, oropharyngeal cancer, testicular cancer, thymus cancer, thyroid cancer, melanoma, and bone cancer. In a further embodiment, the cancer is a metastatic cancer, e.g., metastatic breast cancer. In a further embodiment, the cancer is a metastatic breast cancer comprising metastasis of breast cancer to the lung, brain, bone, pancreas, lymph nodes, and/or liver.

### BRIEF DESCRIPTION OF THE FIGURES

FIG. 1A is a graph showing recovery of active NE, as measured by the NE kinetic assay and expressed as concentration of active NE normalized to the volume of originating whole blood, after lysis of WBC pellets with 0.02% Triton® X-100 (IUPAC name: 2-[4-(2,4,4-trimethylpentan-2-yl)phenoxy]ethanol) Lysis Buffer (0.02% Triton), 1% Triton® X-100 Lysis Buffer (1% Triton), Abcam Lysis Buffer (Abcam), 10% Triton® X-100 Lysis Buffer during a second lysis step following Abcam Lysis Buffer during the first lysis step (10% Triton post-Abcam), or combined recovery of active NE with Abcam Lysis Buffer during the first lysis step and 10% Triton® X-100 Lysis Buffer during the second lysis step (combined). The formulations of the above-mentioned lysis buffers are shown in Table 1A.

FIG. 1B is a graph showing recovery of active PR3, as measured by the PR3 kinetic assay and expressed as concentration of active PR3 normalized to the volume of originating whole blood, after lysis of WBC pellets with 0.02% Triton® X-100 Lysis Buffer (0.02% Triton), 1% Triton® X-100 Lysis Buffer (1% Triton), Abcam Lysis Buffer (Abcam), 10% Triton® X-100 Lysis Buffer during a second lysis step following Abcam Lysis Buffer during the first lysis step (10% Triton post-Abcam), or combined recovery of active PR3 with Abcam Lysis Buffer during the first lysis step and 10% Triton® X-100 Lysis Buffer during the second lysis step (combined). The formulations of the above-mentioned lysis buffers are shown in Table 1A.

FIG. 2A is a graph showing the concentrations of active NE recovered in cell lysates, as measured by the NE kinetic assay and normalized to the volume of originating whole blood, following multi-extractions of NE from WBC pellets of sample groups A-C, or single extractions of NE from WBC pellets of sample groups D and E. In each data set of sample groups A-C, five bars from left to right represent the active NE concentrations in 1°, 2°, 3°, 1°+2°, and 1°+2°+3° cell lysates, respectively. The single bars of sample groups D and E represent the active NE concentrations in 1° cell lysates.

FIG. 2B is a graph showing the concentrations of active PR3 recovered in cell lysates, as measured by the PR3 kinetic assay and normalized to the volume of originating whole blood, following multi-extractions of PR3 from WBC pellets of sample groups A-C, or single extractions of PR3 from WBC pellets of sample groups D and E. In each data set of sample groups A-C, five bars from left to right represent the active PR3 concentrations in 1°, 2°, 3° 1°+2°, and 1°+2°+3° cell lysates, respectively. The single bars of sample groups D and E represent the active PR3 concentrations in 1° cell lysates.

FIG. 2C is a graph showing the concentrations of active NE recovered in 1° cell lysates, as measured by the NE kinetic assay and normalized to the volume of originating whole blood, after single lysis of WBC pellets with 0.02% Triton® X-100 Lysis Buffer (0.02% Triton), Abcam Lysis Buffer (Abcam), 10% Triton® X-100 Lysis Buffer (10% Triton), or after single lysis of a pre-lysis washed WBC pellet with NP-40 Lysis Buffer (NP-40 (washed)). The formulations of the above-mentioned lysis buffers are shown in Table 1A, with NP-40 Lysis Buffer containing 50 mM HEPES buffer, 0.75M NaCl, and 0.05% (v/v) Nonidet® P-40 (IUPAC name: octylphenoxypolyethoxyethanol).

FIG. 2D is a graph showing the concentrations of active PR3 recovered in 1° cell lysates, as measured by the PR3 kinetic assay and normalized to the volume of originating whole blood, after single lysis of WBC pellets with 0.02% Triton® X-100 Lysis Buffer (0.02% Triton), Abcam Lysis Buffer (Abcam), 10% Triton® X-100 Lysis Buffer (10% Triton), or after single lysis of a pre-lysis washed WBC pellet with NP-40 Lysis Buffer (NP-40 (washed)). The formulations of the above-mentioned lysis buffers are shown in Table 1A, with NP-40 Lysis Buffer containing 50 mM HEPES buffer, 0.75M NaCl, and 0.05% (v/v) Nonidet® P-40.

FIG. 2E is a graph showing the concentrations of active NE (normalized to the volume of originating whole blood) recovered from an unwashed WBC pellet and a pre-lysis washed WBC pellet following single lysis using NP-40 Lysis Buffer containing 50 mM HEPES buffer, 0.75M NaCl, and 0.05% (v/v) Nonidet® P-40.

FIG. 2F is a graph showing the concentrations of active PR3 (normalized to the volume of originating whole blood) recovered from an unwashed WBC pellet and a pre-lysis washed WBC pellet following single lysis using NP-40 Lysis Buffer containing 50 mM HEPES buffer, 0.75M NaCl, and 0.05% (v/v) Nonidet® P-40.

FIG. 3A is a graph showing total concentrations of active NE (normalized to the volume of originating whole blood) recovered in wash fraction, 1° cell lysate, and 2° cell lysate, following pre-lysis wash and double extractions of washed WBC pellets with NP-40 Lysis Buffer (at lysis step 1) followed by 10% Triton® X-100 Lysis Buffer (at lysis step 2), NP-40 Lysis Buffer at both lysis steps 1 and 2, or 10% Triton® X-100 Lysis Buffer at both lysis steps 1 and 2. The formulations of the above-mentioned lysis buffers are shown in Table 1A, with NP-40 Lysis Buffer containing 50 mM HEPES buffer, 0.75M NaCl, and 0.05% (v/v) Nonidet® P-40.

FIG. 3B is a graph showing total concentrations of active PR3 (normalized to the volume of originating whole blood) recovered in wash fraction, 1° cell lysate, and 2° cell lysate, following pre-lysis wash and double extractions of washed WBC pellets with NP-40 Lysis Buffer (at lysis step 1) followed by 10% Triton® X-100 Lysis Buffer (at lysis step 2), NP-40 Lysis Buffer at both lysis steps 1 and 2, or 10% Triton® X-100 Lysis Buffer at both lysis steps 1 and 2. The formulations of the above-mentioned lysis buffers are shown in Table 1A, with NP-40 Lysis Buffer containing 50 mM HEPES buffer, 0.75M NaCl, and 0.05% (v/v) Nonidet® P-40.

FIG. 4A is a graph showing individual and total concentrations of active NE (normalized to the volume of originating whole blood) in wash fractions (wash) and 1°, 2°, and 3° cell lysates recovered from control half WBC pellets of four different donors B01-B04. In each donor data set, five bars from left to right represent the individual active NE concentrations in wash, 1°, 2°, 3° cell lysates, and the total active NE concentration, respectively.

FIG. 4B is a graph showing individual and total concentrations of active PR3 (normalized to the volume of originating whole blood) in wash fractions (wash) and 1°, 2°, and 3° cell lysates recovered from control half WBC pellets of four different donors B01-B04. In each donor data set, five bars from left to right represent the individual active PR3 concentrations in the wash, 1°, 2°, 3° cell lysates, and the total active PR3 concentration, respectively.

FIG. 4C is a graph showing individual and total concentrations of active NE (normalized to the volume of originating whole blood) in wash fractions (wash) and 1° and 2° cell lysates recovered from half WBC pellets with enhanced agitation of four different donors B01-B04. In each donor data set, four bars from left to right represent the individual active NE concentrations in wash, 1°, 2° cell lysates, and the total active NE concentration, respectively.

FIG. 4D is a graph showing individual and total concentrations of active PR3 (normalized to the volume of originating whole blood) in wash fractions (wash) and 1° and 2° cell lysates recovered from half WBC pellets with enhanced agitation of four different donors B01-B04. In each donor data set, four bars from left to right represent the individual active PR3 concentrations in wash, 1°, 2° cell lysates, and the total active PR3 concentration, respectively.

FIG. 4E is a graph showing side-by-side comparisons of the total concentrations of active NE (normalized to the volume of originating whole blood) recovered from control half WBC pellets of four different donors B01-B04, as previously shown in FIG. 4A, to the total concentrations of active NE (normalized to the volume of originating whole blood) recovered from half WBC pellets with enhanced agitation of the same donors, as previously shown in FIG. 4C. In each donor data set, the left and right bars represent the total active NE concentrations recovered from control half pellet (control) and half pellet with enhanced agitation (enhanced agitation), respectively.

FIG. 4F is a graph showing side-by-side comparisons of the total concentrations of active PR3 (normalized to the volume of originating whole blood) recovered from control half WBC pellets of four different donors B01-B04, as previously shown in FIG. 4B, to the total concentrations of active PR3 (normalized to the volume of originating whole blood) recovered from half WBC pellets with enhanced agitation of the same donors, as previously shown in FIG. 4D. In each donor data set, the left and right bars represent the total active PR3 concentrations recovered from control half pellet (control) and half pellet with enhanced agitation (enhanced agitation), respectively.

FIG. 5A is a graph showing the concentrations of active NE (normalized to the volume of originating whole blood) recovered in 1° cell lysates following the first step of lysis of pre-lysis washed WBC pellets from two different donors B05 and B04, using NP-40 Lysis Buffer under enhanced agitation (left bar in each donor data set), as compared to the concentrations of active NE (normalized to the volume of originating whole blood) recovered in 1° cell lysates following single-step lysis of unwashed WBC pellets from the same donors, using 0.02% Triton® X-100 Lysis Buffer with half the amount of agitation (right bar in each donor data set). The formulations of the above-mentioned lysis buffers are shown in Table 1A, with NP-40 Lysis Buffer containing 50 mM HEPES buffer, 0.75M NaCl, and 0.05% (v/v) Nonidet® P-40.

FIG. 5B is a graph showing the concentrations of active PR3 (normalized to the volume of originating whole blood) recovered in 1° cell lysates following the first step of lysis of pre-lysis washed WBC pellets from two different donors B05 and B04, using NP-40 Lysis Buffer under enhanced agitation (left bar in each donor data set), as compared to the concentrations of active PR3 (normalized to the volume of originating whole blood) recovered in 1° cell lysates following single-step lysis of unwashed WBC pellets from the same donors, using 0.02% Triton® X-100 Lysis Buffer with half the amount of agitation (right bar in each donor data set). The formulations of the above-mentioned lysis buffers are shown in Table 1A, with NP-40 Lysis Buffer containing 50 mM HEPES buffer, 0.75M NaCl, and 0.05% (v/v) Nonidet® P-40.

FIG. 5C is a graph showing individual concentrations of active NE (normalized to the volume of originating whole blood) in wash fractions (wash), 1°, 2°, 3°, 4°, and 5° cell lysates, as well as total active NE concentrations (normalized to the volume of originating whole blood) of wash+1°, 2°, 3°, 4°, 5° cell lysates and subtotal active NE concentrations (normalized to the volume of originating whole blood) of wash+1°, 2°, 3° cell lysates, recovered from duplicate donor WBC pellet samples (B05a and B05b), via pre-lysis wash and a five-step repeated pellet lysis process using NP-40 Lysis Buffer (containing 50 mM HEPES buffer, 0.75M NaCl, and 0.05% (v/v) Nonidet® P-40) under enhanced agitation. In each data set, the left bar represents the active NE concentration recovered from donor pellet sample B05a, and the right bar from donor pellet sample B05b.

FIG. 5D is a graph showing individual concentrations of active PR3 (normalized to the volume of originating whole blood) in wash fractions (wash), 1°, 2°, 3°, 4°, and 5° cell lysates, as well as total active PR3 concentrations (normalized to the volume of originating whole blood) of wash+1°, 2°, 3°, 4°, 5° cell lysates and subtotal active PR3 concentrations (normalized to the volume of originating whole blood) of wash+1°, 2°, 3° cell lysates, recovered from duplicate donor WBC pellet samples (B05a and B05b), via pre-lysis wash and a five-step repeated pellet lysis process using NP-40 Lysis Buffer (containing 50 mM HEPES buffer, 0.75M NaCl, and 0.05% (v/v) Nonidet® P-40) under enhanced agitation. In each data set, the left bar represents the active PR3 concentration recovered from donor pellet sample B05a, and the right bar from donor pellet sample B05b.

FIG. 5E is a graph showing total concentrations of active NE (normalized to the volume of originating whole blood) recovered from unwashed WBC pellets of two different donors (B05 and B04), via single-step lysis using 0.02% Triton® X-100 Lysis Buffer with agitation (left bar in each donor data set), as compared to subtotal active NE concentrations (normalized to the volume of originating whole blood) of wash+1°, 2°, 3° cell lysates (middle bar in each donor data set, labeled as “NP40 (3 extracts)”) and total active NE concentrations (normalized to the volume of originating whole blood) of wash+1°, 2°, 3°, 4° 5° cell lysates (right bar in each donor data set, labeled as “NP40 (5 extracts)”), recovered from WBC pellets of the same donors, via pre-lysis wash and a five-step repeated pellet lysis process using NP-40 Lysis Buffer under enhanced (twice the amount of) agitation. The formulations of the above-mentioned lysis buffers are shown in Table 1A, with NP-40 Lysis Buffer containing 50 mM HEPES buffer, 0.75M NaCl, and 0.05% (v/v) Nonidet® P-40.

FIG. 5F is a graph showing total concentrations of active PR3 (normalized to the volume of originating whole blood) recovered from unwashed WBC pellets of two different donors (B05 and B04), via single-step lysis using 0.02% Triton® X-100 Lysis Buffer with agitation (left bar in each donor data set), as compared to subtotal active PR3 concentrations (normalized to the volume of originating whole blood) of wash+1°, 2°, 3° cell lysates (middle bar in each donor data set, labeled as “NP40 (3 extracts)”) and total active PR3 concentrations (normalized to the volume of originating whole blood) of wash+1°, 2°, 3°, 4°, 5° cell lysates (right bar in each donor data set, labeled as “NP40 (5 extracts)”), recovered from WBC pellets of the same donors, via pre-lysis wash and a five-step repeated pellet lysis process using NP-40 Lysis Buffer under enhanced (twice the amount of) agitation. The formulations of the above-mentioned lysis buffers are shown in Table 1A, with NP-40 Lysis Buffer containing 50 mM HEPES buffer, 0.75M NaCl, and 0.05% (v/v) Nonidet® P-40.

FIG. 6A is a graph showing individual concentrations of active NE (normalized to the volume of originating whole blood) in wash fractions (wash), 1°, 2°, 3°, 4° and 5° cell lysates recovered from donor B04 WBC pellet, via pre-lysis wash and a five-step repeated pellet lysis process using NP-40 Lysis Buffer (containing 50 mM HEPES buffer, 0.75M NaCl, and 0.05% (v/v) Nonidet® P-40) under enhanced agitation. In each data set, the left bar and right bar represent the active NE concentrations determined by the NE kinetic assay without an antifoam (NO AF) and with an antifoam (AF), respectively.

FIG. 6B is a graph showing individual concentrations of active PR3 (normalized to the volume of originating whole blood) in wash fractions (wash), 1°, 2°, 3°, 4° and 5° cell lysates recovered from donor B04 WBC pellet, via pre-lysis wash and a five-step repeated pellet lysis process using NP-40 Lysis Buffer (containing 50 mM HEPES buffer, 0.75M NaCl, and 0.05% (v/v) Nonidet® P-40) under enhanced agitation. In each data set, the left bar and right bar represent the active PR3 concentrations determined by the PR3 kinetic assay without an antifoam (NO AF) and with an antifoam (AF), respectively.

FIG. 6C is a graph showing individual concentrations of active NE (normalized to the volume of originating whole blood) in wash fractions (wash), 1°, 2°, 3, 4° and 5° cell lysates recovered from donor B05 WBC pellet, via pre-lysis wash and a five-step repeated pellet lysis process using NP-40 Lysis Buffer (containing 50 mM HEPES buffer, 0.75M NaCl, and 0.05% (v/v) Nonidet® P-40) under enhanced agitation. In each data set, the left bar and right bar represent the active NE concentrations determined by the NE kinetic assay without an antifoam (NO AF) and with an antifoam (AF), respectively.

FIG. 6D is a graph showing individual concentrations of active PR3 (normalized to the volume of originating whole blood) in wash fractions (wash), 1°, 2°, 3, 4° and 5° cell lysates recovered from donor B05 WBC pellet, via pre-lysis wash and a five-step repeated pellet lysis process using NP-40 Lysis Buffer (containing 50 mM HEPES buffer, 0.75M NaCl, and 0.05% (v/v) Nonidet® P-40) under enhanced agitation. In each data set, the left bar and right bar represent the active PR3 concentrations determined by the PR3 kinetic assay without an antifoam (NO AF) and with an antifoam (AF), respectively.

FIG. 7A is a graph showing at various sample timepoints recovery of active NE, as measured by the NE kinetic assay and expressed as concentration of active NE normalized to the volume of originating whole blood, in cell lysates prepared from pre-lysis washed WBC pellets subjected to a three-step repeated pellet lysis process using NP-40 Lysis Buffer (containing 50 mM HEPES buffer, 0.75M NaCl, and 0.05% (v/v) Nonidet® P-40) with enhanced agitation. At each sample timepoint, the left bar (Individual Samples) represents the recovery of active NE determined by assaying individual cell lysate samples for NE activity and summing the individual activity, while the right bar (Pooled Sample) represents the recovery of active NE determined by pooling equal volumes of individual cell lysates to obtain a pooled sample and assaying the NE activity of the pooled sample.

FIG. 7B is a graph showing at various sample timepoints recovery of active PR3, as measured by the PR3 kinetic assay and expressed as concentration of active PR3 normalized to the volume of originating whole blood, in cell lysates prepared from pre-lysis washed WBC pellets subjected to a three-step repeated pellet lysis process using NP-40 Lysis Buffer (containing 50 mM HEPES buffer, 0.75M NaCl, and 0.05% (v/v) Nonidet® P-40) with enhanced agitation. At each sample timepoint, the left bar (Individual Samples) represents the recovery of active PR3 determined by assaying individual cell lysate samples for PR3 activity and summing the individual activity, while the right bar (Pooled Sample) represents the recovery of active PR3 determined by pooling equal volumes of individual cell lysates to obtain a pooled sample and assaying the PR3 activity of the pooled sample.

FIG. 8 is a graph showing total concentrations of active CatG (represented by bars) recovered from groups A-G WBC pellets obtained from five different donors (Donors 1-5) and processed and lysed under various conditions, with the line graph showing the averages of the total concentrations of active CatG among the five donors in each group. The concentrations of active CatG were quantified using the kinetic CatG assay with Suc-AAPF-pNA peptide from Sigma as the substrate and normalized to the volume of originating whole blood.

FIG. 9 is a graph showing individual concentrations and their mathematical sums of active CatG in 1°, 2° and 3° cell lysates (represented by stacked bars) recovered from group E WBC pellets obtained from five different donors (Donors 1-5) using NP-40 Lysis Buffer (containing 50 mM HEPES buffer, 0.75M NaCl, and 0.05% (v/v) Nonidet® P-40), as well as active CatG concentrations of pooled lysates (represented by the line graph) obtained by combining equal volumes of 1°, 2°, and 3° cell lysates derived from each donor WBC pellet. “Average” represents a hypothetical donor having the corresponding average values of the five donors. The concentrations of active CatG were quantified using the kinetic CatG assay with Suc-AAPF-pNA peptide from Sigma as the substrate and normalized to the volume of originating whole blood.

FIG. 10 is a graph showing total concentrations of active CatG (represented by bars) recovered from groups A-G WBC pellets obtained from five different donors (Donors 1-5) and processed and lysed under various conditions, with the line graph showing the averages of the total concentrations of active CatG among the five donors in each group. The concentrations of active CatG were quantified using the ELISA-based ProteaseTag® Active CatG Immunoassay from ProAxsis and normalized to the volume of originating whole blood.

FIG. 11 is a graph showing individual concentrations and their mathematical sums of active CatG in 1°, 2° and 3° cell lysates (represented by stacked bars) recovered from group E WBC pellets obtained from five different donors (Donors 1-5) using NP-40 Lysis Buffer (containing 50 mM HEPES buffer, 0.75M NaCl, and 0.05% (v/v) Nonidet® P-40), as well as active CatG concentrations of pooled lysates (represented by the line graph) obtained by combining equal volumes of 1°, 2°, and 3° cell lysates derived from each donor WBC pellet. “Average” represents a hypothetical donor having the corresponding average values of the five donors. The concentrations of active CatG were quantified using the ELISA-based ProteaseTag® Active CatG Immunoassay from ProAxsis and normalized to the volume of originating whole blood.

FIG. 12 is a schematic summary of an exemplary method for extracting NSPs from a whole blood sample.

FIG. 13A is a graph showing the change from baseline in the concentration of active NE in the sputum samples obtained from patients of the three treatment arms. *, p<0.05 versus placebo.

FIG. 13B is a graph showing the change from baseline in the concentration of active PR3 in the sputum samples obtained from patients of the three treatment arms. *, p<0.05 versus placebo.

FIG. 13C is a graph showing the change from baseline in the concentration of active CatG in the sputum samples obtained from patients of the three treatment arms. *, p<0.05 versus placebo.

FIG. 14A is a graph showing the change from baseline in the concentration of active NE in WBCs derived from the whole blood samples of patients of the three treatment arms. The concentrations of active NE were determined by the kinetic NE assay and normalized to the volume of originating whole blood. *, p<0.05 versus placebo.

FIG. 14B is a graph showing the change from baseline in the concentration of active PR3 in WBCs derived from the whole blood samples of patients of the three treatment arms. The concentrations of active PR3 were determined by the kinetic PR3 assay and normalized to the volume of originating whole blood. *, p<0.05 versus placebo.

### DETAILED DESCRIPTION OF THE INVENTION

Throughout the present disclosure, the term “about” may be used in conjunction with numerical values and/or ranges. The term “about” is understood to mean those values near to a recited value. For example, “about 40 [units]” may mean within ±25% of 40 (e.g., from 30 to 50), within ±20%, ±15%, ±10%, 9%, ±8%, ±7%, ±6%, ±5%, ±4%, ±3%, ±2%, ±1%, less than ±1%, or any other value or range of values therein or there below.

Throughout the present disclosure, numerical ranges are provided for certain quantities. It is to be understood that these ranges comprise all subranges therein. Thus, the range “from 50 to 80” includes all possible ranges therein (e.g., from 51 to 79, from 52 to 78, from 53 to 77, from 54 to 76, from 55 to 75, from 60 to 70, etc.). Furthermore, all values within a given range may be an endpoint for the range encompassed thereby (e.g., the range of from 50 to 80 includes the ranges with endpoints such as from 55 to 80, from 50 to 75, etc.).

The activity of one or more NSPs, proportional to the amounts or concentrations of mature, active forms of NSPs, may underlie or correlate with a certain diseases state or treatment. As such, extraction of NSPs from a patient blood sample and determination of the concentrations of active forms of NSPs may be critical for the diagnosis and treatment of certain diseases where the DPP1/NSP cascade is implicated. The present application provides an efficient and reproducible method for extracting NSPs from a sample comprising white blood cells obtained from a subject. Additionally, the present application provides methods of treating a DPP1-mediated condition in a patient with reversible DPP1 inhibitors. The treatment methods provided herein harness the concentration of an active form of one or more NSPs extracted from a patient's white blood cell sample as a biomarker to guide the selection of, or adjustment to, an effective dosage of one or more of the reversible DPP1 inhibitors provided herein.

In one aspect, the present disclosure provides an efficient and reproducible method for extracting one or more NSPs from a sample obtained from a subject, wherein the sample comprises white blood cells. The method includes:
contacting the sample with a first aqueous medium comprising at least 0.01% (v/v) of a first surfactant to obtain a first cell lysate comprising a first NSP extract, and a first WBC residual, wherein the first NSP extract comprises the one or more NSPs,
separating the first cell lysate from the first WBC residual, to provide a first separated cell lysate comprising the first NSP extract,
contacting the first WBC residual with a second aqueous medium comprising at least 0.010% (v/v) of a second surfactant to obtain a second cell lysate comprising a second NSP extract, and a second WBC residual, wherein the second NSP extract comprises the one or more NSPs, and
separating the second cell lysate from the second WBC residual to provide a second separated cell lysate comprising the second NSP extract.

The sample in one embodiment, is obtained from a subject. In a further embodiment, the subject is a human subject. In another embodiment, the subject is a mammal. In a further embodiment, the mammal is selected from a domesticated animal (e.g., cow, sheep, cat, dog, and horse), primate (e.g., human and non-human primates such as a monkey), rabbit, or a rodent (e.g., mouse, rat).

NSPs reside inside the azurophilic granules of neutrophils and are implicated in the regulation of inflammatory conditions. Non-limiting exemplary NSPs include neutrophil elastase (NE), proteinase 3 (PR3), cathepsin G (CatG), and NSP4. In one embodiment, the method disclosed herein is performed to extract NE, PR3, CatG, NSP4, or a combination thereof. In another embodiment, the method disclosed herein is performed to extract NE. In another embodiment, the method disclosed herein is performed to extract PR3. In another embodiment, the method disclosed herein is performed to extract NE and PR3. In another embodiment, the method disclosed herein is performed to extract CatG. In another embodiment, the method disclosed herein is performed to extract NE, PR3, and CatG. In another embodiment, the method disclosed herein is performed to extract NSP4. The one or more NSPs extracted from the sample according to the disclosed methods include all forms of the NSPs present in the sample, including active as well as inactive forms.

In one embodiment of an extraction method provided herein, the sample comprising white blood cells (WBCs) comprises a cell suspension comprising WBCs. In another embodiment, the sample comprising WBCs comprises a WBC pellet. In one embodiment, the sample comprising WBCs derives from a whole blood sample and is substantially devoid of red blood cells, e.g., through selective lysis of red blood cells.

In one embodiment of an extraction method provided herein, the WBCs in the sample are washed with an aqueous wash solution before the sample is contacted with the first aqueous medium to lyse the WBCs. In one embodiment, the aqueous wash solution is a phosphate buffered saline (PBS) solution. In another embodiment, the aqueous wash solution is a saline solution comprising about 0.9% NaCl. In another embodiment, the aqueous wash solution comprises a Tris-based alkaline buffer and NaCl, e.g., an aqueous solution comprising about 100 mM Tris and about 100 mM NaCl with a pH of about 7.5. The pre-lysis wash of WBCs may be accomplished by adding the aqueous wash solution to the sample, e.g., a sample comprising a WBC suspension or pellet, optionally followed by gentle mixing to facilitate the washing. The gentle mixing for washing may be carried out by low intensity pipetting, vortexing, shaking, stirring of the mixture of the aqueous wash solution and sample, for example, using a stirring rod or on a stir plate with stir bar, or with a paddle, such as a United States Pharmacopeia (USP) apparatus 2 (paddle). The mixture of the aqueous wash solution and the sample may then be centrifuged to provide a supernatant (also referred to as “wash fraction”) and a pellet comprising the WBCs. In some embodiments, the supernatant (wash fraction) comprises the one or more NSPs and thus is collected for determination of the concentration of an active form of the one or more NSPs indicative of NSP activity. With the supernatant collected, the pellet comprising the WBCs is contacted with the first aqueous medium.

Any aqueous medium disclosed herein, e.g., the first and second aqueous medium comprising the first and second surfactants, respectively, as described above, and a third, a fourth, a fifth, and a sixth aqueous medium comprising the respective third, fourth, fifth, and sixth surfactants described below, contains a buffer and a surfactant, with the surfactant dissolved in the buffer. Suitable buffers include, but are not limited to, phosphate-buffered saline such as Dulbecco's phosphate-buffered saline (DPBS), a Tris buffer, a Tris-buffered saline (TBS) solution, and a HEPES buffer. In one embodiment, the buffer is a HEPES buffer. In a further embodiment, the HEPES buffer contains about 50 mM HEPES and about 0.75 M NaCl. The aqueous medium may be free, or substantially free (e.g., contains less than about 5%, such as less than about 4%, less than about 3%, less than about 2%, less than about 1%, less than about 0.5%, less than about 0.1%, less than about 0.05%, or less than about 0.01% v/v) of an alcohol, such as, e.g., ethanol.

The surfactant present in any of the aqueous media disclosed herein can be any type of surfactant, such as an ionic surfactant or a nonionic surfactant. The surfactant, in one embodiment, is a nonionic surfactant. Non-limiting examples of suitable nonionic surfactants for use in the aqueous media disclosed herein include nonionic esters, such as ethylene glycol esters, propylene glycol esters, glyceryl esters, polyglyceryl esters, sorbitan esters, sucrose esters, and ethoxylated esters, nonionic alkanolamides and ethers, such as fatty alcohol ethoxylates, propoxylated alcohols, ethoxylated/propoxylated block polymers, and polyoxyethylene surfactants. In a further embodiment, the surfactant used in the aqueous media disclosed herein is a nonionic polyoxyethylene surfactant comprising a hydrophilic polyethylene oxide chain. In even a further embodiment, the surfactant comprising a hydrophilic polyethylene oxide chain further comprises an aromatic hydrocarbon group that is lipophilic or hydrophobic.

In one embodiment, the nonionic polyoxyethylene surfactant in any of the aqueous media disclosed herein comprises octylphenoxypolyethoxyethanol (sold under the trade names Nonidet® P-40 or IGEPAL® CA-630 available from Sigma Aldrich of St. Louis, MO). In a further embodiment, one or more of the aqueous media disclosed herein, e.g., the first, second, third, fourth, fifth, or sixth aqueous medium, or a combination thereof, comprises about 0.05% (v/v) of octylphenoxypolyethoxyethanol, about 0.75 M NaCl, and about 50 mM HEPES.

In another embodiment, the nonionic polyoxyethylene surfactant comprises 2-[4-(2,4,4-trimethylpentan-2-yl)phenoxy]ethanol (also known as octylphenol ethoxylate, available as Triton® X-100 from Sigma Aldrich of St. Louis, MO).

In another embodiment, the nonionic polyoxyethylene surfactant comprises polyoxyethylene (9) nonylphenylether (branched) (available as NP-40 from ThermoFisher Scientific, or as IGEPAL® CO-630 from Sigma).

In another embodiment, the nonionic polyoxyethylene surfactant comprises polyoxyethylene (20) sorbitan monolaurate (available under the trade name Tween® 20).

In one embodiment, the surfactant has a critical micelle concentration (CMC) less than about 5 mM, less than about 2 mM, or less than about 1 mM. For instance, the surfactant may have a CMC of about 0.1 mM, 0.2 mM, 0.3 mM, 0.4 mM, or 0.5 mM, or a CMC ranging from about 0.1 to about 1 mM, such as from about 0.1 to about 0.5 mM.

According to the NSP extraction methods disclosed herein, the sample comprising WBCs is contacted with the first aqueous medium comprising a first surfactant at a first lysis step, at which the WBCs in the sample, or a portion thereof, are lysed by the first surfactant, resulting in extraction of one or more NSPs from the WBCs and formation of the first cell lysate containing the extracted NSPs and other components of the WBCs soluble in the first aqueous medium (referred to herein as “the first NSP extract” comprising the one or more NSPs). The first WBC residual also forms following the first lysis step. The first WBC residual, in one embodiment, contains components of the WBCs incapable of being solubilized by the first surfactant in the first lysis step, e.g., the cytoskeleton, as well as remaining NSPs not yet extracted. The first WBC residual may also contain a portion of the WBCs not yet lysed following the first lysis step. To achieve a more complete extraction of NSPs, the first cell lysate is separated from the first WBC residual by, for example, settling or centrifugation, to provide the first separated cell lysate comprising the first NSP extract.

The first separated cell lysate, in one embodiment, is collected for measuring a concentration of an active form of the one or more NSPs indicative of NSPs activity, and/or pooling with subsequent cell lysates generated by the methods provided herein. The first WBC residual, in one embodiment, is subjected to a further (second) lysis step by contacting the first WBC residual with the second aqueous medium to obtain the second cell lysate containing additional NSPs extracted (i.e., the second NSP extract comprising the one or more NSPs) and the second WBC residual. In one embodiment, the second WBC residual, similar to the first WBC residual, contains components of the WBCs in the sample incapable of being solubilized by the second surfactant, as well as WBCs not yet lysed so far and/or NSPs that may remain still.

Further extraction of NSPs is contemplated by the methods provided herein. For example, to achieve an even further extraction of NSPs, upon separating the second cell lysate from the second WBC residual to obtain the second separated cell lysate comprising the second NSP extract, the second WBC residual, in one embodiment, is subjected to an additional lysis step (i.e., a third lysis step) via contacting with a third aqueous medium comprising at least 0.01% (v/v) of a third surfactant to obtain a third cell lysate containing NSPs further extracted (i.e., a third NSP extract comprising the one or more NSPs) and a successor (third) WBC residual, followed by separation of the third cell lysate from the third WBC residual to provide a third separated cell lysate comprising the third NSP extract. As the third WBC residual may still contain NSPs not yet extracted and/or WBCs not yet lysed, one, two, three, or more additional repeated lysis steps (i.e., a fourth lysis step with a fourth aqueous medium comprising at least 0.01% (v/v) of a fourth surfactant, a fifth lysis step with a fifth aqueous medium comprising at least 0.01% (v/v) of a fifth surfactant, a sixth lysis step with a sixth aqueous medium comprising at least 0.01% (v/v) of a sixth surfactant, or beyond) can be performed, with each additional lysis step generating a successor WBC residual (e.g., a fourth, a fifth, and a sixth residual) and a new cell lysate containing further extracted NSP (e.g., a fourth, fifth, and sixth cell lysate comprising the respective fourth, fifth, and sixth NSP extract, with each NSP extract comprising the one or more NSPs). The new cell lysate is then separated from the corresponding WBC residual to provide a new separated cell lysate (e.g., a fourth, fifth, and sixth separated cell lysate comprising the respective fourth, fifth, and sixth NSP extract, with each NSP extract comprising the one or more NSPs). In one embodiment, the third WBC residual is contacted with the fourth aqueous medium to obtain a fourth cell lysate comprising a fourth NSP extract, and a fourth WBC residual, followed by separation of the fourth cell lysate from the fourth WBC residual to provide a fourth separated cell lysate comprising the fourth NSP extract. In a further embodiment, the fourth WBC residual is contacted with the fifth aqueous medium to obtain a fifth cell lysate comprising a fifth NSP extract, and a fifth WBC residual, followed by separation of the fifth cell lysate from the fifth WBC residual to provide a fifth separated cell lysate comprising the fifth NSP extract. In a further embodiment, the fifth WBC residual is contacted with the sixth aqueous medium to obtain a sixth cell lysate comprising a sixth NSP extract, and a sixth WBC residual, followed by separation of the sixth cell lysate from the sixth WBC residual to provide a sixth separated cell lysate comprising the sixth NSP extract.

Where multiple, repeated lysis steps are carried out, in one embodiment, the surfactant present in an aqueous medium used for each lysis step can be the same or different. Additionally, the concentration of the surfactant present in an aqueous medium used for each lysis step can be the same or different, regardless of the identity of the surfactant. For example, when a WBC sample is subjected to 6-step repeated lysis, in one embodiment, the first, second, third, fourth, fifth, and sixth surfactants in their respective aqueous medium are the same surfactant, present at the same concentration in all the six aqueous media, or present at different concentrations in at least two of the six aqueous media. In another embodiment, at least two of the first, second, third, fourth, fifth, and sixth surfactants are different surfactants. Regardless of the identity of the surfactant in each aqueous medium, the concentrations of the surfactants among the six aqueous media may be the same. Alternatively, the concentrations of the surfactants in at least two of the six aqueous media are different.

In another embodiment where multiple, repeated lysis steps are carried out, the aqueous medium used for each lysis step can be the same or different. In one embodiment, all of the aqueous media used are the same aqueous medium. In another embodiment, at least two of the aqueous media used are different aqueous media. For example, when a WBC sample is subjected to 6-step repeated lysis, in one embodiment, the first, second, third, fourth, fifth, and sixth aqueous media are the same aqueous medium. In another embodiment, at least two of the first, second, third, fourth, fifth, and sixth aqueous media are different aqueous media. For instance, the first aqueous medium and the sixth aqueous medium may be different aqueous media and the remaining aqueous media are the same as the first or sixth aqueous medium. Alternatively, the first, third, and sixth aqueous media may be different from one another, and the remaining aqueous media are the same as any one of the first, third, and sixth aqueous medium.

Each of the aqueous media, e.g., a first, a second, a third, a fourth, a fifth, and a sixth aqueous medium described in the present application, for lysing the WBCs, a portion thereof and/or a WBC residual (or a portion thereof) to generate cell lysates containing one or more extracted NSPs, comprises at least 0.01% (v/v) of its corresponding surfactant. In one embodiment, the first, second, third, fourth, fifth, or sixth aqueous medium, or a combination thereof comprises at least 0.02% (v/v) of the respective first, second, third, fourth, fifth, or sixth surfactant. In another embodiment, the first, second, third, fourth, fifth, or sixth aqueous medium, or a combination thereof comprises at least 0.05% (v/v) of the respective first, second, third, fourth, fifth, or sixth surfactant. In another embodiment, the first, second, third, fourth, fifth, or sixth aqueous medium, or a combination thereof comprises from about 0.02% (v/v) to about 1.5% (v/v) of the respective first, second, third, fourth, fifth, or sixth surfactant. In another embodiment, the first, second, third, fourth, fifth, or sixth aqueous medium, or a combination thereof comprises from about 0.03% (v/v) to about 1% (v/v) of the respective first, second, third, fourth, fifth, or sixth surfactant. In another embodiment, the first, second, third, fourth, fifth, or sixth aqueous medium, or a combination thereof comprises from about 0.04% (v/v) to about 0.8% (v/v) of the respective first, second, third, fourth, fifth, or sixth surfactant. In another embodiment, the first, second, third, fourth, fifth, or sixth aqueous medium, or a combination thereof comprises from about 0.05% (v/v) to about 0.6% (v/v) of the respective first, second, third, fourth, fifth, or sixth surfactant. In another embodiment, the first, second, third, fourth, fifth, or sixth aqueous medium, or a combination thereof comprises about 0.05% (v/v) of the respective first, second, third, fourth, fifth, or sixth surfactant. In another embodiment, the first, second, third, fourth, fifth, or sixth aqueous medium, or a combination thereof comprises the respective first, second, third, fourth, fifth, or sixth surfactant at a concentration that is above its critical micelle concentration (CMC). When present at each of the above-mentioned concentrations in the first, second, third, fourth, fifth, or sixth aqueous medium, or a combination thereof, in one embodiment, the respective first, second, third, fourth, fifth, or sixth surfactant, or a combination thereof, is a nonionic surfactant. In a further embodiment, the respective first, second, third, fourth, fifth, or sixth surfactant, or a combination thereof, is a nonionic polyoxyethylene surfactant. In still a further embodiment, the respective first, second, third, fourth, fifth, or sixth surfactant, or a combination thereof, is octylphenoxypolyethoxyethanol.

In some embodiments, each lysis step is carried out at a relatively low temperature to stabilize NSPs extracted from the WBCs or a WBC residual. In one embodiment, the sample comprising WBCs is contacted with the first aqueous medium at a temperature of from about 0° C. to about 10° C., from about 2° C. to about 8° C., from about 3° C. to about 6° C., or about 4° C. In another embodiment, a WBC residual (e.g., a first, second, third, fourth, or fifth WBC residual) is contacted with a corresponding aqueous medium at a temperature of from about 0° C. to about 10° C., from about 2° C. to about 8° C., from about 3° C. to about 6° C., or about 4° C.

In one embodiment, contacting the sample with the first aqueous medium at the first lysis step includes mixing the sample with the first aqueous medium. In another embodiment where the WBCs in the sample are washed with an aqueous wash solution before the sample comprising a WBC pellet is contacted with the first aqueous medium, contacting the WBC pellet comprising the washed WBCs with the first aqueous medium includes mixing the WBC pellet with the first aqueous medium. In a further embodiment, mixing the sample or the WBC pellet with the first aqueous medium includes agitating the sample or the WBC pellet with the first aqueous medium. Likewise, at each of the subsequent additional lysis steps, contacting a WBC residual (e.g., a first, second, third, fourth, or fifth WBC residual) with a corresponding aqueous medium may include mixing the WBC residual with the corresponding aqueous medium. In a further embodiment, mixing the WBC residual with the corresponding aqueous medium includes agitating the WBC residual with the corresponding aqueous medium. Agitation mentioned above may be effected by, for example, pipetting, vortexing, shaking, stirring by different means (e.g., by using a stirring rod or a stir plate with stir bar), or by using a paddle, such as a USP apparatus 2 (paddle). In one embodiment, the agitation is effected by pipetting, for example, from about 10 to about 30 times, from about 15 to about 25 times, or about 20 times during each lysis step.

NSPs present in the wash fraction and in individual separated cell lysates can be detected and quantified by various methods, including western blotting using an anti-NSP antibody, ELISA assays, and enzymatic activity assays. Exemplary ELISA assays include ProteaseTag® Active NE Immunoassay, ProteaseTag® Active PR3 Immunoassay, and ProteaseTag® Active CatG Immunoassay from ProAxsis (Belfast, Northern Ireland) described in the Examples set forth herein. Exemplary enzymatic activity assays include NE, PR3, and CatG enzymatic kinetic assays, also described in the Examples.

In one embodiment, an active form of an NSP present in the wash fraction and/or in individual separated cell lysates (e.g., a first, second, third, fourth, fifth, or sixth separated cell lysate) is detected by measuring the enzymatic activity of the NSP, or the concentration of the active form of the NSP. Because the enzymatic activity of an NSP can be converted to the concentration of an active form of the NSP using a standard curve, as described in the Examples, references to NSP activity and references to the concentration of an active form of an NSP are interchangeable in the present application. In another embodiment, the enzymatic activity of an NSP, or the concentration of the active form of an NSP, in each separated cell lysate (e.g., a first, second, third, fourth, fifth, or sixth separated cell lysate) is measured individually. The total NSP activity, or total concentration of the active form of the NSP, of all the separated cell lysates, in one embodiment, can be calculated as the mathematic sum of the individual activity or concentrations. In another embodiment, two or more separated cell lysates are combined to provide a pooled cell lysate comprising a pooled NSP extract. The total NSP activity, or the total concentration of the active form of an NSP, of all the separated cell lysates can be calculated based on (1) the NSP activity, or the concentration of the active form of the NSP, measured with the pooled cell lysate and (2) the NSP activity, or the concentrations of the active form of the NSP, measured individually with the remaining non-pooled, separated cell lysates. In still another embodiment, all of the separated cell lysates are combined to provide a single pooled cell lysate, with the total NSP activity, or total concentration of the active form of an NSP, of all the separated cell lysates obtained based on the measurement of the NSP activity, or the concentration of the active form of the NSP of the single pooled cell lysate. In one embodiment, equal volumes of two or more separate cell lysates are combined to provide a pooled cell lysate.

In another aspect, the present disclosure relates to a method of treating a DPP1-mediated condition in a patient in need thereof. The method includes:
(a) measuring a baseline concentration of an active form of one or more NSPs extracted from a first sample comprising white blood cells obtained from the patient,
(b) orally administering to the patient daily for a first administration period of about 2 weeks to about 16 weeks, a pharmaceutical composition comprising a first daily dosage of about 10 mg to about 40 mg of a compound of formula (I), or a pharmaceutically acceptable salt thereof,

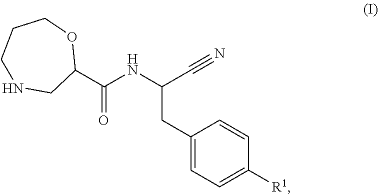

wherein,
R¹ is

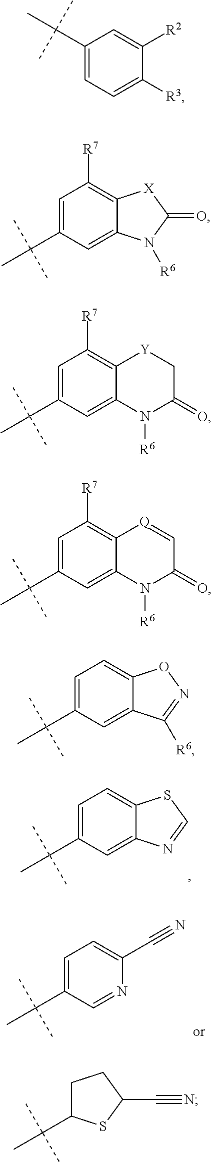

R² is hydrogen, F, Cl, Br, OSO₂C₁₋₃alkyl, or C₁₋₃alkyl;
R³ is hydrogen, F, Cl, Br, CN, CF₃, SO₂C₁₋₃alkyl, CONH₂ or SO₂NR⁴R⁵,
wherein R⁴ and R⁵ together with the nitrogen atom to which they are attached form an azetidine, pyrrolidine or piperidine ring;
X is O, S or CF₂;
Y is O or S;
Q is CH or N;
R⁶ is C₁₋₃alkyl, wherein the C₁₋₃alkyl is optionally substituted by 1, 2 or 3 F and optionally by one substituent selected from OH, OC₁₋₃alkyl, N(C₁₋₃alkyl)₂, cyclopropyl, or tetrahydropyran; and
R⁷ is hydrogen, F, Cl or CH₃;
(c) measuring a concentration of the active form of the one or more NSPs extracted from a second sample comprising white blood cells, wherein the second sample is obtained from the patient during the first administration period, or about one week or less subsequent to the first administration period,
(d) comparing the concentration from the second sample with the baseline concentration from the first sample; and
if the concentration from the second sample is reduced by about 10% or more as compared to the baseline concentration from the first sample, then orally administering to the patient daily for a second administration period the same daily dosage as the first daily dosage of the compound of formula (I), or a pharmaceutically acceptable salt thereof, or
if the concentration from the second sample is not reduced by about 10% or more as compared to the baseline concentration from the first sample, then orally administering to the patient daily for a second administration period a second daily dosage of the compound of formula (I), or a pharmaceutically acceptable salt thereof, wherein the second daily dosage is about 1.5 times to about 7 times the first daily dosage.

The lysosomal cysteine dipeptidyl peptidase 1 (DPP1) is the proteinase that activates NSPs, including NE, PR3, CatG, and NSP4, by removal of the N-terminal dipeptide sequences from their precursors during azurophilic granule assembly. See Pham et al., J Immunol. 173:7277-7281 (2004); Pham et al., Nature Reviews Immunology, 6:541-550 (2006); Perera et al, PNAS, 109:6229-6234 (2012), each of which is incorporated herein by reference in its entirety for all purposes. The compounds of formula (I) and their pharmaceutically acceptable salts are reversible inhibitors of DPP1 activity. Unless otherwise provided herein, the daily dosage amount of a compound of formula (I), or a pharmaceutically acceptable salt thereof provided herein is for the respective free base form of the compound of formula (I).

As used herein, “C₁₋₃” means a carbon group having 1, 2 or 3 carbon atoms.

The term “alkyl”, unless otherwise noted, includes both straight and branched chain alkyl groups and may be, substituted or non-substituted. “Alkyl” groups include, but are not limited to, methyl, ethyl, n-propyl, i-propyl, butyl, pentyl.

The term “pharmaceutically acceptable”, unless otherwise noted, is used to characterize a moiety (e.g., a salt, dosage form, or excipient) as being appropriate for use in accordance with sound medical judgment. In general, a pharmaceutically acceptable moiety has one or more benefits that outweigh any deleterious effect that the moiety may have. Deleterious effects may include, for example, excessive toxicity, irritation, allergic response, and other problems and complications.

The term “treating” in one embodiment, includes: (1) preventing or delaying the appearance of clinical symptoms of the state, disorder or condition developing in the patient that may be afflicted with or predisposed to the state, disorder or condition but does not yet experience or display clinical or subclinical symptoms of the state, disorder or condition; (2) inhibiting the state, disorder or condition (i.e., arresting, reducing or delaying the development of the disease, or a relapse thereof in case of maintenance treatment, of at least one clinical or subclinical symptom thereof); (3) relieving the condition (i.e., causing regression of the state, disorder or condition or at least one of its clinical or subclinical symptoms); and (4) prophylaxis against a disease state, disorder or condition.

In the treatment methods disclosed herein, the one or more NSPs may be extracted from the first sample and/or the second sample according to the NSP extraction methods disclosed in the present application. The NSPs may also be extracted using other known methods.

In the treatment methods disclosed herein, the first sample, with which the baseline concentration of an active form of one or more NSPs is measured, is obtained from the patient before the patient is administered the pharmaceutical composition for the first time, i.e., prior to the first administration period.

In one embodiment of the methods, the first administration period is about 2 weeks to about 12 weeks. In another embodiment, the first administration period is about 2 weeks to about 8 weeks. In another embodiment, the first administration period is about 3 weeks to about 6 weeks. In another embodiment, the first administration period is about 3 weeks to about 5 weeks. In another embodiment, the first administration period is about three weeks. In another embodiment, the first administration period is about four weeks. In another embodiment, the first administration period is about five weeks. In another embodiment, the first administration period is about 6 weeks. In another embodiment, the first administration period is about 7 weeks. In another embodiment, the first administration period is about 8 weeks. In another embodiment, the first administration period is about 9 weeks. In another embodiment, the first administration period is about 10 weeks. In another embodiment, the first administration period is about 11 weeks. In another embodiment, the first administration period is about 12 weeks.

In one embodiment of the methods, the second sample is obtained from the patient during the first administration period. For example, the second sample may be obtained from the patient at the end of the first administration period, or about 1, 2, 3, 4, 5, 6, or 7 days before the end of the first administration period. In a further embodiment, the first administration period is about four weeks.

In one embodiment of the methods, the second sample is obtained from the patient about one week subsequent to the first administration period. In other embodiments, the second sample is obtained from the patient about 1, 2, 3, 4, 5, 6, or 7 days subsequent to the first administration period. In a further embodiment, the first administration period is about four weeks.

In one embodiment of the methods, the first administration period is about 4 weeks, and the second sample is obtained from the patient at about 4 weeks during the first administration period.

In one embodiment of the methods, the one or more NSPs comprise PR3, and the concentrations of the active form of PR3 from the first and second samples are measured and compared.

In one embodiment, the one or more NSPs comprise CatG, and the concentrations of the active form of CatG from the first and second samples are measured and compared.

In one embodiment, the one or more NSPs comprise NSP4, and the concentrations of the active form of NSP4 from the first and second samples are measured and compared.

In one embodiment of the methods, the one or more NSPs comprise NE, and the concentrations of the active form of NE from the first and second samples are measured and compared. In a further embodiment, if the concentration of the active form of NE from the second sample is reduced by about 19% or more as compared to the baseline concentration of the active form of NE from the first sample, then the compound of formula (I), or a pharmaceutically acceptable salt thereof is administered daily and orally at the same daily dosage as the first daily dosage for the second administration period, and if the concentration of the active form of NE from the second sample is not reduced by about 19% or more as compared to the baseline concentration of the active form of NE from the first sample, then the compound of formula (I), or a pharmaceutically acceptable salt thereof is administered daily and orally at the second daily dosage for the second administration period.

The treatment methods disclosed herein use as a biomarker a reduction in the concentration of an active form of an NSP extracted from a patient's white blood cell (WBC) sample obtained during or subsequent the first administration period. This biomarker guides the determination of the daily dosage of the compound of formula (I), or a pharmaceutically acceptable salt thereof administered for the second administration period. If the biomarker, i.e., the reduction in the concentration of active NSP extracted from the patient's WBC sample, reaches or exceeds a certain threshold as defined above, the patient is administered the compound of formula (I), or a pharmaceutically acceptable salt thereof for a second administration period at the same daily dosage as that during the first administration period, i.e., the first daily dosage. Otherwise, the patient is administered the compound of formula (I), or a pharmaceutically acceptable salt thereof for a second administration period a second daily dosage that is higher than the first daily dosage defined above.

In one embodiment, the first daily dosage of the compound of formula (I), or a pharmaceutically acceptable salt thereof is about 10 mg to about 25 mg. In another embodiment, the first daily dosage of the compound of formula (I), or a pharmaceutically acceptable salt thereof is about 10 mg to about 15 mg. In another embodiment, the first daily dosage of the compound of formula (I), or a pharmaceutically acceptable salt thereof is about 10 mg to about 12 mg. In another embodiment, the first daily dosage of the compound of formula (I), or a pharmaceutically acceptable salt thereof is about 16 mg to about 25 mg. In another embodiment, the first daily dosage of the compound of formula (I), or a pharmaceutically acceptable salt thereof is about 20 mg to about 25 mg. In another embodiment, the first daily dosage of the compound of formula (I), or a pharmaceutically acceptable salt thereof is about 25 mg to about 40 mg.

In one embodiment, the second daily dosage is about 1.5 times to about 6 times the first daily dosage. In a further embodiment, the first daily dosage is about 10 mg to about 15 mg, or about 10 mg to about 12 mg.

In another embodiment, the second daily dosage is about 1.5 times to about 5 times the first daily dosage. In a further embodiment, the first daily dosage is about 10 mg to about 15 mg, or about 10 mg to about 12 mg.

In another embodiment, the second daily dosage is about 1.5 times to about 4 times the first daily dosage. In a further embodiment, the first daily dosage is about 10 mg to about 15 mg, about 10 mg to about 12 mg, or about 16 mg to about 25 mg.

In another embodiment, the second daily dosage is about 1.5 times to about 3 times the first daily dosage. In a further embodiment, the first daily dosage is about 16 mg to about 25 mg, about 20 mg to about 25 mg, or about 25 mg to about 40 mg.

In another embodiment, the second daily dosage is about 1.5 times to about 2 times the first daily dosage. In a further embodiment, the first daily dosage is about 16 mg to about 25 mg, about 20 mg to about 25 mg, or about 25 mg to about 40 mg.

In one embodiment, the first daily dosage of the compound of formula (I), or a pharmaceutically acceptable salt thereof is about 10 mg, and the second daily dosage is about 2 times to about 6.5 times the first daily dosage. In another embodiment, the first daily dosage of the compound of formula (I), or a pharmaceutically acceptable salt thereof is about 25 mg, and the second daily dosage is about 1.6 times to about 2.6 times the first daily dosage.

In one embodiment of the treatment methods, the second administration period is at least 1 month, e.g., from about 1 month to about 24 months, from about 1 month to about 12 months, from about 5 months to about 24 months, from about 5 months to about 18 months, or from about 5 months to about 15 months. In another embodiment, the second administration period is from about 3 months to about 6 months. In another embodiment, the second administration period is from about 6 months to about 12 months. In another embodiment, the second administration period is from about 12 months to about 18 months. In yet another embodiment, the second administration period is from about 12 months to about 24 months.

In one embodiment, the pharmaceutical composition is administered orally to the patient once daily during the first and second administration periods to reach the first and second daily dosages of the compound of formula (I), or a pharmaceutically acceptable salt thereof, respectively. In another embodiment, the pharmaceutical composition is administered orally to the patient twice daily during the first and second administration periods to reach the first and second daily dosages of the compound of formula (I), or a pharmaceutically acceptable salt thereof, respectively.

Embodiments of the compounds of formula (I), or pharmaceutically acceptable salts thereof, that can be used according to the methods for treating a DPP1-mediated condition disclosed herein are described below. It is noted that one or more DPP1 inhibitors other than the compounds of formula (I), or pharmaceutically acceptable salts thereof, may also be used in place of, or in combination with, the compounds of formula (I), or pharmaceutically acceptable salts thereof, according to the disclosed treatment methods. Non-limiting examples of DPP1 inhibitors other than the compounds of formula (I), or pharmaceutically acceptable salts thereof contemplated for use include those disclosed in Miller et al., “Epithelial desquamation observed in a phase I study of an oral cathepsin C inhibitor (GSK2793660),” Br J Clin Pharmacol. 83:2813-2820 (2017); Methot N et al., “Inhibition of the activation of multiple serine proteases with a cathepsin C inhibitor requires sustained exposure to prevent proenzyme processing,” J. Biol Chem. 282:20836-20846 (2007); Guay D et al., “Design and synthesis of dipeptidyl nitriles as potent, selective, and reversible inhibitors of cathepsin C,” Bioorg Med Chem Lett. 19:5392-5396 (2009); Methot N et al., “In Vivo Inhibition of Serine Proteases Processing Requires a High Fractional Inhibition of Cathepsin C,” Mol. Pharm. 73:1857-1865 (2008); Guay D et al., “Therapeutic Utility and Medicinal chemistry of Cathepsin C Inhibitors,” Curr Top Med Chem. 10:708-716 (2010); Bondebjerg J et al., “Novel semicarbazide-derived inhibitors of human dipeptidyl peptidase I (hDPPI),” Bioorg Med Chem. 13:4408-4424 (2005); Bondejberg J et al., “Dipeptidyl Nitriles as Human Dipeptidyl Peptidase 1 Inhibitors,” Bioorg Med Chem Lett. 16:3614-3617 (2006); Guarino C et al., “Prolonged pharmacological inhibition of cathepsin C results in elimination of neutrophil serine proteases,” Biochem Pharmacol. 131:52-67 (2017); U.S. Pat. Nos. 8,871,783, 8,877,775, 8,889,708, 8,987,249, 8,999,975, 9,073,869, 9,440,960, 9,713,606, 9,879,026, RE47,636E1, 10,238,633, 9,856,228, and 10,479,781, each of which is incorporated herein by reference in its entirety for all purposes.

### Compounds of Formula (I)

In one embodiment of the treatment methods disclosed herein, the compound of formula (I) is an S,S diastereomer. In other words, the compound of formula (I) has the following stereochemistry:

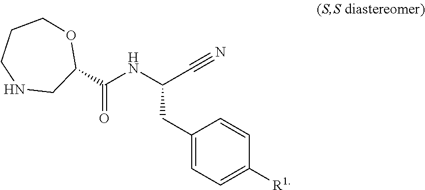

The other diastereomeric forms are also contemplated. For example, in one embodiment, the compound of formula (I) is the R,R diastereomer:

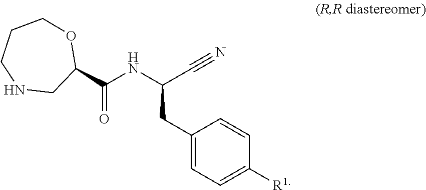

In another embodiment, the compound of formula (I) is the R,S diastereomer:

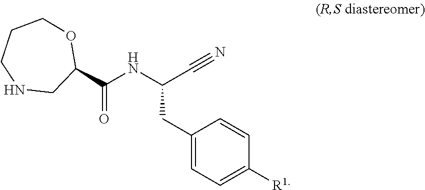

In even another embodiment, the compound of formula (I) is the S,R diastereomer:

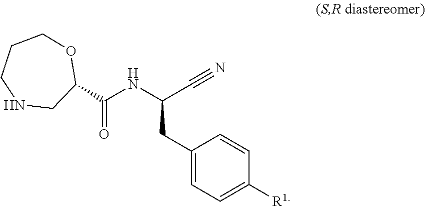

In one embodiment, the composition comprises a mixture of an S,S diastereomer of a compound of formula (I) and an S,R diastereomer of a compound of formula (I).

In one embodiment, the composition comprises a mixture of an S,S diastereomer of a compound of formula (I) and an R,S diastereomer of a compound of formula (I).

In one embodiment, the composition comprises a mixture of an S,S diastereomer of a compound of formula (I) and an R,R diastereomer of a compound of formula (I).

In one embodiment, R¹ is

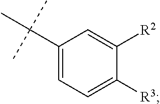

R² is hydrogen, F, Cl, Br, OSO₂C₁₋₃alkyl, or C₁₋₃alkyl; R³ is hydrogen, F, Cl, Br, CN, CF₃, SO₂C₁₋₃alkyl, CONH₂ or SO₂NR⁴R⁵, wherein R⁴ and R⁵ together with the nitrogen atom to which they are attached form an azetidine, pyrrolidine or piperidine ring. In a further embodiment, R² is hydrogen, F, Cl or C₁₋₃alkyl; and R³ is hydrogen, F, Cl, CN or SO₂C₁₋₃alkyl. In a further embodiment, R³ is hydrogen, F or CN.

In another embodiment, R¹ is

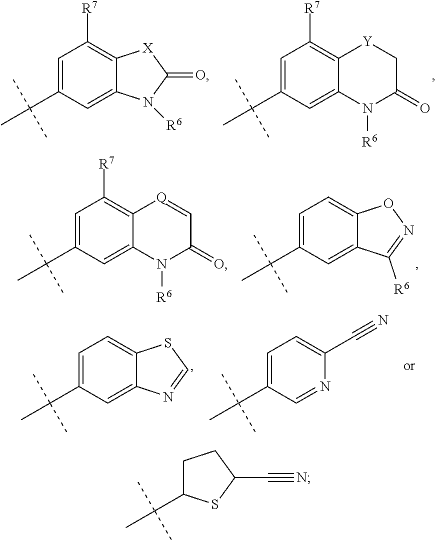

X is O, S or CF₂; Y is O or S; Q is CH or N; R⁶ is C₁₋₃alkyl, wherein the C₁₋₃alkyl is optionally substituted by 1, 2 or 3 F and optionally substituted by OH, OC₁₋₃alkyl, N(C₁₋₃alkyl)₂, cyclopropyl, or tetrahydropyran; and R⁷ is hydrogen, F, Cl or CH₃. In a further embodiment, R¹ is

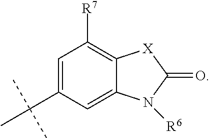

In another embodiment, R¹ is

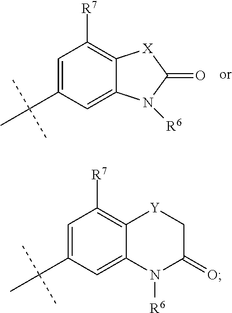

X is O, S or CF₂; Y is O or S; R⁶ is C₁₋₃alkyl, optionally substituted by 1, 2 or 3 F and optionally substituted by OH, OC₁₋₃alkyl, N(C₁₋₃alkyl)₂, cyclopropyl, or tetrahydropyran; and R⁷ is hydrogen, F, Cl or CH₃. In a further embodiment, R¹ is

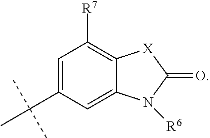

In another embodiment, R¹ is

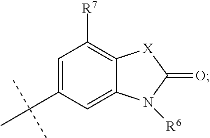

X is O, S or CF₂; R⁶ is C₁₋₃alkyl, wherein the C₁₋₃alkyl is optionally substituted by 1, 2 or 3 F; and R⁷ is hydrogen, F, Cl or CH₃.

In another embodiment, R¹ is

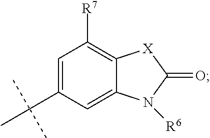

X is O; R⁶ is C₁₋₃alkyl, wherein the C₁₋₃alkyl is optionally substituted by 1, 2 or 3 F; and R⁷ is hydrogen. In a further embodiment, R⁶ is C₁₋₃alkyl, i.e., methyl, ethyl, or propyl. In still a further embodiment, R⁶ is methyl.

In one embodiment, R² is hydrogen, F, Cl, Br, OSO₂C₁₋₃alkyl or C₁₋₃alkyl.

In a further embodiment, R² is hydrogen, F, Cl or C₁₋₃alkyl.

In still a further embodiment, R² is hydrogen, F or C₁₋₃alkyl.

In one embodiment, R³ is hydrogen, F, Cl, Br, CN, CF₃, SO₂C₁₋₃alkyl CONH₂ or SO₂NR⁴R⁵, wherein R⁴ and R⁵ together with the nitrogen atom to which they are attached form an azetidine, pyrrolidine or piperidine ring.

In a further embodiment, R³ is hydrogen, F, Cl, CN or SO₂C₁₋₃alkyl.

In still a further embodiment, R³ is hydrogen, F or CN.

In one embodiment, R⁶ is C₁₋₃alkyl, wherein the C₁₋₃alkyl is optionally substituted by 1, 2 or 3 F and optionally by one substituent selected from OH, OC₁₋₃alkyl, N(C₁₋₃alkyl)₂, cyclopropyl, or tetrahydropyran.

In a further embodiment, R⁶ is C₁₋₃alkyl, wherein the C₁₋₃alkyl is optionally substituted by 1, 2 or 3 F. In still a further embodiment, R⁶ is methyl or ethyl. In still a further embodiment, R⁶ is methyl.

In one embodiment, R⁷ is hydrogen, F, Cl or CH₃. In a further embodiment R⁷ is hydrogen.

In one embodiment, the compound of formula (I) is (2S)—N-{(1S)-1-cyano-2-[4-(3-methyl-2-oxo-2,3-dihydro-1,3-benzoxazol-5-yl)phenyl]ethyl}-1,4-oxazepane-2-carboxamide (brensocatib):

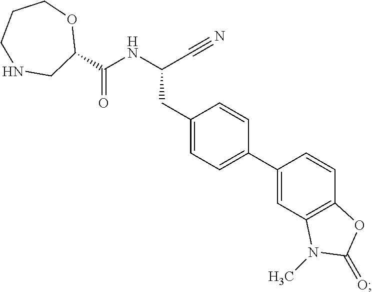 [SMILES: Cn1c(=O)oc2ccc(-c3ccc(C[C@@H](C#N)NC(=O)[C@@H]4CNCCCO4)cc3)cc21]

or a pharmaceutically acceptable salt thereof. In a further embodiment, the compound of formula (I) is brensocatib.

In one embodiment, the compound of formula (I) is:
(2S)—N-[(1S)-1-Cyano-2-(4′-cyanobiphenyl-4-yl)ethyl]-1,4-oxazepane-2-carboxamide,
(2S)—N-{(1S)-1-Cyano-2-[4-(3-methyl-2-oxo-2,3-dihydro-1,3-benzoxazol-5-yl)phenyl]ethyl}-1,4-oxazepane-2-carboxamide,
(2S)—N-{(1S)-1-Cyano-2-[4-(3,7-dimethyl-2-oxo-2,3-dihydro-1,3-benzoxazol-5-yl)phenyl]ethyl}-1,4-oxazepane-2-carboxamide,
4′-[(2S)-2-Cyano-2-{[(2S)-1,4-oxazepan-2-ylcarbonyl]amino}ethyl]biphenyl-3-yl methanesulfonate,
(2S)—N-{(1S)-1-Cyano-2-[4-(3-methyl-1,2-benzoxazol-5-yl)phenyl]ethyl}-1,4-oxazepane-2-carboxamide,
(2S)—N-{(1S)-1-Cyano-2-[4′-(trifluoromethyl)biphenyl-4-yl]ethyl}-1,4-oxazepane-2-carboxamide,
(2S)—N-[(1S)-1-Cyano-2-(3′,4′-difluorobiphenyl-4-yl)ethyl]-1,4-oxazepane-2-carboxamide,
(2S)—N-{(1S)-1-Cyano-2-[4-(6-cyanopyridin-3-yl)phenyl]ethyl}-1,4-oxazepane-2-carboxamide,
(2S)—N-{(1S)-1-Cyano-2-[4-(4-methyl-3-oxo-3,4-dihydro-2H-1,4-benzothiazin-6-yl)phenyl]ethyl}-1,4-oxazepane-2-carboxamide,
(2S)—N-{(1S)-1-Cyano-2-[4-(3-ethyl-7-methyl-2-oxo-2,3-dihydro-1,3-benzoxazol-5-yl)phenyl]ethyl}-1,4-oxazepane-2-carboxamide,
(2S)—N-[(1S)-1-Cyano-2-{4-[3-(2-hydroxy-2-methylpropyl)-2-oxo-2,3-dihydro-1,3-benzoxazol-5-yl]phenyl}ethyl]-1,4-oxazepane-2-carboxamide,
(2S)—N-[(1S)-1-Cyano-2-{4-[3-(2,2-difluoroethyl)-7-fluoro-2-oxo-2,3-dihydro-1,3-benzoxazol-5-yl]phenyl}ethyl]-1,4-oxazepane-2-carboxamide,
(2S)—N-[(1S)-1-Cyano-2-(4-{3-[2-(dimethylamino)ethyl]-2-oxo-2,3-dihydro-1,3-benzoxazol-5-yl}phenyl)ethyl]-1,4-oxazepane-2-carboxamide,
(2S)—N-{(1S)-1-Cyano-2-[4-(3,3-difluoro-1-methyl-2-oxo-2,3-dihydro-1H-indol-6-yl)phenyl]ethyl}-1,4-oxazepane-2-carboxamide,
(2S)—N-{(1S)-1-Cyano-2-[4-(7-fluoro-3-methyl-2-oxo-2,3-dihydro-1,3-benzoxazol-5-yl)phenyl]ethyl}-1,4-oxazepane-2-carboxamide,
(2S)—N-{(1S)-1-Cyano-2-[4-(3-ethyl-2-oxo-2,3-dihydro-1,3-benzoxazol-5-yl)phenyl]ethyl}-1,4-oxazepane-2-carboxamide,
(2S)—N-[(1S)-1-Cyano-2-{4-[3-(cyclopropylmethyl)-2-oxo-2,3-dihydro-1,3-benzoxazol-5-yl]phenyl}ethyl]-1,4-oxazepane-2-carboxamide,
(2S)—N-[(1S)-1-Cyano-2-{4-[3-(2-methoxyethyl)-2-oxo-2,3-dihydro-1,3-benzothiazol-5-yl]phenyl}ethyl]-1,4-oxazepane-2-carboxamide,
(2S)—N-[(1S)-1-Cyano-2-{4-[2-oxo-3-(propan-2-yl)-2,3-dihydro-1,3-benzoxazol-5-yl]phenyl}ethyl]-1,4-oxazepane-2-carboxamide,
(2S)—N-{(1S)-1-Cyano-2-[4-(4-methyl-3-oxo-3,4-dihydro-2H-1,4-benzoxazin-6-yl)phenyl]ethyl}-1,4-oxazepane-2-carboxamide,
(2S)—N-[(1S)-1-Cyano-2-{4-[3-(2-methoxyethyl)-2-oxo-2,3-dihydro-1,3-benzoxazol-5-yl]phenyl}ethyl]-1,4-oxazepane-2-carboxamide,
(2S)—N-{(1S)-1-Cyano-2-[4-(5-cyanothiophen-2-yl)phenyl]ethyl}-1,4-oxazepane-2-carboxamide,
(2S)—N-[(1S)-2-(4′-Carbamoyl-3′-fluorobiphenyl-4-yl)-1-cyanoethyl]-1,4-oxazepane-2-carboxamide,
(2S)—N-{(1S)-1-Cyano-2-[4-(1-methyl-2-oxo-1,2-dihydroquinolin-7-yl)phenyl]ethyl}-1,4-oxazepane-2-carboxamide,
(2S)—N-[(1S)-1-Cyano-2-{4-[2-oxo-3-(tetrahydro-2H-pyran-4-ylmethyl)-2,3-dihydro-1,3-benzoxazol-5-yl]phenyl}ethyl]-1,4-oxazepane-2-carboxamide,
(2S)—N-{(1S)-2-[4-(7-Chloro-3-methyl-2-oxo-2,3-dihydro-1,3-benzoxazol-5-yl)phenyl]-1-cyanoethyl}-1,4-oxazepane-2-carboxamide,
(2S)—N-[(1S)-1-Cyano-2-{4-[3-(2,2-difluoroethyl)-2-oxo-2,3-dihydro-1,3-benzoxazol-5-yl]phenyl}ethyl]-1,4-oxazepane-2-carboxamide,
(2S)—N-[(1S)-1-Cyano-2-{4-[2-oxo-3-(2,2,2-trifluoroethyl)-2,3-dihydro-1,3-benzoxazol-5-yl]phenyl}ethyl]-1,4-oxazepane-2-carboxamide,
(2S)—N-{(1S)-1-Cyano-2-[4-(3-methyl-2-oxo-2,3-dihydro-1,3-benzothiazol-5-yl)phenyl]ethyl}-1,4-oxazepane-2-carboxamide,
(2S)—N-{(1S)-1-Cyano-2-[4′-(methylsulfonyl)biphenyl-4-yl]ethyl}-1,4-oxazepane-2-carboxamide,
(2S)—N-{(1S)-2-[4′-(Azetidin-1-ylsulfonyl)biphenyl-4-yl]-1-cyanoethyl}-1,4-oxazepane-2-carboxamide,
(2S)—N-[(1S)-1-Cyano-2-(4′-fluorobiphenyl-4-yl)ethyl]-1,4-oxazepane-2-carboxamide,
(2S)—N-{(1S)-2-[4-(1,3-Benzothiazol-5-yl)phenyl]-1-cyanoethyl}-1,4-oxazepane-2-carboxamide, or
(2S)—N-[(1S)-1-Cyano-2-(4′-cyanobiphenyl-4-yl)ethyl]-1,4-oxazepane-2-carboxamide,

or a pharmaceutically acceptable salt of one of the foregoing compounds.

In one embodiment, the compound of formula (I) is brensocatib. In some embodiments, brensocatib is in polymorphic Form A as disclosed in U.S. Pat. No. 9,522,894, the disclosure of which is incorporated herein by reference in its entirety for all purposes. In some embodiments, brensocatib is characterized by an X-ray powder diffraction pattern having a peak at about 12.2±0.2 (° 2-theta), measured using CuKα radiation. In some embodiments, brensocatib is characterized by an X-ray powder diffraction pattern having a peak at about 20.6±0.2 (° 2-theta), measured using CuKα radiation. In some embodiments, brensocatib is characterized by an X-ray powder diffraction pattern having a peak at about 12.2±0.2 and about 20.6±0.2 (° 2-theta), measured using CuKα radiation. In some embodiments, brensocatib is characterized by an X-ray powder diffraction pattern having a peak at about 12.2±0.2, about 14.3±0.2, about 16.2±0.2, about 19.1±0.2 and about 20.6±0.2 (° 2-theta), measured using CuKα radiation.

As provided throughout, according to the methods provided herein, a compound of formula (I) can be administered as a pharmaceutically acceptable salt. A pharmaceutically acceptable salt of a compound of formula (I) may be advantageous due to one or more of its chemical or physical properties, such as stability in differing temperatures and humidities, or a desirable solubility in H₂O, oil, or other solvent. In some instances, a salt may be used to aid in the isolation or purification of the compound of formula (I).

Where the compound of formula (I) is sufficiently acidic, pharmaceutically acceptable salts include, but are not limited to, an alkali metal salt, e.g., Na or K, an alkali earth metal salt, e.g., Ca or Mg, or an organic amine salt. Where the compound of formula (I) is sufficiently basic, pharmaceutically acceptable salts include, but are not limited to, inorganic or organic acid addition salts.

There may be more than one cation or anion depending on the number of charged functions and the valency of the cations or anions.

For reviews on suitable salts, and pharmaceutically acceptable salts amenable for use herein, see Berge et al., J Pharm. Sci., 1977, 66, 1-19 or “Handbook of Pharmaceutical Salts: Properties, selection and use”, P. H. Stahl, P. G. Vermuth, IUPAC, Wiley-VCH, 2002, incorporated by reference herein in its entirety for all purposes.

The compounds of formula (I) may form mixtures of its salt and co-crystal forms. It is also to be understood that the methods provided herein can employ such salt/co-crystal mixtures of the compound of formula (I).

Salts and co-crystals may be characterized using well known techniques, for example X-ray powder diffraction, single crystal X-ray diffraction (for example to evaluate proton position, bond lengths or bond angles), solid state NMR, (to evaluate for example, C, N or P chemical shifts) or spectroscopic techniques (to measure for example, O—H, N—H or COOH signals and IR peak shifts resulting from hydrogen bonding).

It is also to be understood that compounds of formula (I) may exist in solvated form, e.g., hydrates, including solvates of a pharmaceutically acceptable salt of a compound of formula (I).

In one embodiment, compounds of formula (I) may exist as racemates and racemic mixtures, single enantiomers, individual diastereomers and diastereomeric mixtures. It is to be understood that the present disclosure encompasses all such isomeric forms, e.g., the S,S diastereomer, the S,R diastereomer, the R,S diastereomer, and the R,R diastereomer disclosed herein, as well as a mixture of any two or more of the foregoing diastereomers. Accordingly, in one embodiment, the compound of formula (I) is (2S)—N-{(1S)-1-Cyano-2-[4-(3-methyl-2-oxo-2,3-dihydro-1,3-benzoxazol-5-yl)phenyl]ethyl}-1,4-oxazepane-2-carboxamide (i.e., brensocatib, the S,S isomer), shown below.

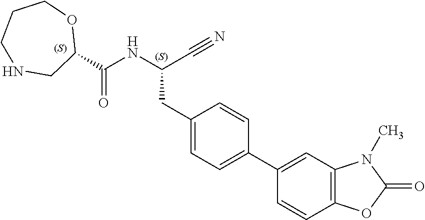

or a pharmaceutically acceptable salt thereof.

In one embodiment, the compound of formula (I) is (2R)—N-{(1R)-1-Cyano-2-[4-(3-methyl-2-oxo-2,3-dihydro-1,3-benzoxazol-5-yl)phenyl]ethyl}-1,4-oxazepane-2-carboxamide (i.e., the R,R isomer), shown below.

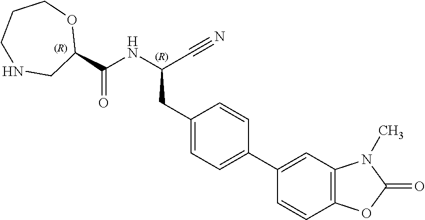

or a pharmaceutically acceptable salt thereof.

In one embodiment, the compound of formula (I) is (2S)—N-{(1R)-1-Cyano-2-[4-(3-methyl-2-oxo-2,3-dihydro-1,3-benzoxazol-5-yl)phenyl]ethyl}-1,4-oxazepane-2-carboxamide (i.e., the S,R isomer), shown below.

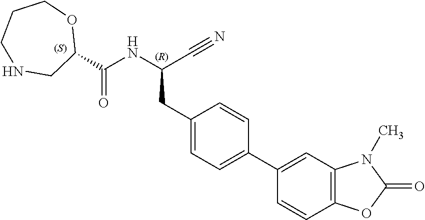

or a pharmaceutically acceptable salt thereof.

In one embodiment, the compound of formula (I) is (2R)—N-{(1S)-1-Cyano-2-[4-(3-methyl-2-oxo-2,3-dihydro-1,3-benzoxazol-5-yl)phenyl]ethyl}-1,4-oxazepane-2-carboxamide (i.e., the R,S isomer), shown below.

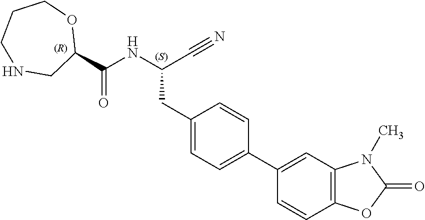

or a pharmaceutically acceptable salt thereof.

In one embodiment, the composition comprises a mixture of two or more of the aforementioned stereoisomers. The mixture in one embodiment, comprises the S,S isomer (brensocatib) and the S,R isomer of a compound of formula (I). In another embodiment, the composition comprises a mixture of the S,S isomer (brensocatib) and the R,S isomer. In yet another embodiment, the composition comprises a mixture of the S,S isomer (brensocatib) and the R,R isomer.

Certain compounds of formula (I) may also contain linkages (e.g. carbon-carbon bonds, carbon-nitrogen bonds such as amide bonds) wherein bond rotation is restricted about that particular linkage, e.g. restriction resulting from the presence of a ring bond or double bond. Accordingly, it is to be understood that the present disclosure encompasses all such isomers. Certain compounds of formula (I) may also contain multiple tautomeric forms. It is to be understood that the present disclosure encompasses all such tautomeric forms. Stereoisomers may be separated using conventional techniques, e.g. chromatography or fractional crystallization, or the stereoisomers may be made by stereoselective synthesis.

In a further embodiment, the compounds of formula (I) encompass any isotopically-labeled (or “radio-labelled”) derivatives of a compound of formula (I). Such a derivative is a derivative of a compound of formula (I) wherein one or more atoms are replaced by an atom having an atomic mass or mass number different from the atomic mass or mass number typically found in nature. Examples of radionuclides that may be incorporated include ²H (also written as “D” for deuterium). As such, in one embodiment, a compound of formula (I) is provided where one or more hydrogen atoms are replaced by one or more deuterium atoms; and the deuterated compound is used in one of the methods provided herein for treating a DPP1-mediated condition.

The skilled person will recognize that the compounds of formula (I) may be prepared, in known manner, in a variety of ways. For example, in one embodiment, compounds of formula (I) are prepared according to the methods set forth in U.S. Pat. No. 9,522,894, incorporated by reference herein in its entirety for all purposes.

### Pharmaceutical Compositions

The compounds of formula (I), or pharmaceutically acceptable salts thereof, may be used on their own, but will generally be administered in the form of a pharmaceutical composition in which the formula (I) compound/salt (active pharmaceutical ingredient (API)) is in a composition comprising a pharmaceutically acceptable adjuvant(s), diluents(s) and/or carrier(s). Conventional procedures for the selection and preparation of suitable pharmaceutical formulations are described in, for example, “Pharmaceuticals—The Science of Dosage Form Designs”, M. E. Aulton, Churchill Livingstone, 2^{nd} Ed. 2002, incorporated by reference herein in its entirety for all purposes.

Depending on the mode of administration, the pharmaceutical composition may comprise from about 0.05 to about 99 wt %, for example, from about 0.05 to about 80 wt %, or from about 0.10 to about 70 wt %, or from about 0.10 to about 50 wt %, of API, all percentages by weight being based on the total weight of the pharmaceutical composition. Unless otherwise provided herein, API weight percentages provided herein are for the respective free base form of the compound of formula (I).

In one embodiment, the pharmaceutical composition is in the oral dosage form of a film-coated oral tablet. In another embodiment, the oral dosage form is an immediate release dosage form with rapid dissolution characteristics under in vitro test conditions. In one embodiment, the oral dosage form is administered once daily to reach the first and/or second daily dosage disclosed herein. In a further embodiment, the oral dosage form is administered at approximately the same time every day, e.g., prior to breakfast. In another embodiment, the oral dosage form is administered 2× daily to reach the first and/or second daily dosage disclosed herein.

In an oral dosage form, the compound of formula (I) may be admixed with adjuvant(s), diluent(s) or carrier(s), for example, lactose, saccharose, sorbitol, mannitol; starch, for example, potato starch, corn starch or amylopectin; cellulose derivative; binder, for example, gelatine or polyvinylpyrrolidone; disintegrant, for example cellulose derivative, and/or lubricant, for example, magnesium stearate, calcium stearate, polyethylene glycol, wax, paraffin, and the like, and then compressed into tablets. If coated tablets are required, the cores, prepared as described above, may be coated with a suitable polymer dissolved or dispersed in water or readily volatile organic solvent(s). Alternatively, the tablet may be coated with a concentrated sugar solution which may contain, for example, gum arabic, gelatine, talcum and titanium dioxide.

For the preparation of soft gelatine capsules for oral administration, the compound of formula (I) may be admixed with, for example, a vegetable oil or polyethylene glycol. Hard gelatine capsules may contain granules of the compound using pharmaceutical excipients like the above-mentioned excipients for tablets. Also, liquid or semisolid formulations of the compound of formula (I) may be filled into hard gelatine capsules.

In one embodiment, the composition is an oral disintegrating tablet (ODT). ODTs differ from traditional tablets in that they are designed to be dissolved on the tongue rather than swallowed whole.

In one embodiment, the composition is an oral thin film or an oral disintegrating film (ODF). Such formulations, when placed on the tongue, hydrate via interaction with saliva, and releases the active compound from the dosage form. The ODF, in one embodiment, contains a film-forming polymer such as hydroxypropylmethylcellulose (HPMC), hydroxypropyl cellulose (HPC), pullulan, carboxymethyl cellulose (CMC), pectin, starch, polyvinyl acetate (PVA) or sodium alginate.

Liquid preparations for oral administration may be in the form of syrups, solutions or suspensions. Solutions, for example may contain the compound of formula (I), the balance being sugar and a mixture of ethanol, water, glycerol and propylene glycol. Optionally such liquid preparations may contain coloring agents, flavoring agents, saccharine and/or carboxymethylcellulose as a thickening agent. Furthermore, other excipients known to those skilled in art may be used when making formulations for oral use.

In one embodiment of the methods, the pharmaceutical composition is one of the compositions described in International Application Publication No. WO 2019/166626, the disclosure of which is incorporated herein by reference in its entirety for all purposes.

In another embodiment of the methods, the pharmaceutical composition administered to the patient is Composition (A) comprising:
(a) from about 1 to about 30 wt % of the compound of formula (I), or a pharmaceutically acceptable salt thereof;
(b) from about 45 to about 85 wt % of a pharmaceutical diluent;
(c) from about 6 to about 30 wt % of a compression aid;
(d) from about 1 to about 15 wt % of a pharmaceutical disintegrant;
(e) from about 0.00 to about 2 wt % of a pharmaceutical glidant; and
(f) from about 1 to about 10 wt % of a pharmaceutical lubricant;
wherein the components add up to 100 wt %.

In a further embodiment, the compound of formula (I) is brensocatib. In one embodiment, brensocatib is in polymorphic Form A. In another embodiment, brensocatib is characterized by one of the X-ray powder diffraction patterns described above.

In some embodiments of the methods, Composition (A) comprises the compound of formula (I), e.g., brensocatib, in an amount from about 1 to about 25 wt %; from about 1 to about 20 wt %; from about 1 to about 15 wt %; from about 1 to about 10 wt %; from about 1 to about 5 wt %, or from about 1 to about 3 wt % of the total weight of the composition.

In some embodiments of the methods, Composition (A) comprises the compound of formula (I), e.g., brensocatib, in an amount from about 1.5 to about 30 wt %; from about 1.5 to about 25 wt %; from about 1.5 to about 20 wt %; from about 1.5 to about 15 wt %; from about 1.5 to about 10 wt %; or from about 1.5 to about 5 wt % of the total weight of the composition.

In some embodiments of the methods, Composition (A) comprises the compound of formula (I), e.g., brensocatib, in an amount from about 3 to about 30 wt %; from about 3 to about 25 wt %; from about 3 to about 20 wt %; from about 3 to about 15 wt %; from about 3 to about 10 wt %; or from about 3 to about 5 wt % of the total weight of the composition. In a further embodiment, the compound of formula (I) is present at from about 3 to about 10 wt % of the total weight of the composition. In a further embodiment, the compound of formula (I) is brensocatib, or a pharmaceutically acceptable salt thereof.

In some embodiments of the methods, Composition (A) comprises the compound of formula (I), e.g., brensocatib, in an amount of about 1 wt %, about 2 wt %, about 3 wt %, about 4 wt %, about 5 wt %, about 6 wt %, about 7 wt %, about 8 wt %, about 9 wt %, about 10 wt %, about 11 wt %, about 12 wt %, about 13 wt %, about 14 wt %, about 15 wt %, about 16 wt %, about 17 wt %, about 18 wt %, about 19 wt %, about 20 wt %, about 21 wt %, about 22 wt %, about 23 wt %, about 24 wt %, about 25 wt %, about 26 wt %, about 27 wt %, about 28 wt %, about 29 wt % or about 30 wt % of the total weight of the composition.

In some embodiments of the methods, Composition (A) comprises one or more pharmaceutical diluents selected from the group consisting of microcrystalline cellulose, calcium carbonate, calcium phosphate, calcium sulfate, cellulose acetate, erythritol, ethylcellulose, fructose, inulin, isomalt, lactitol, lactose, magnesium carbonate, magnesium oxide, maltitol, maltodextrin, maltose, mannitol, polydextrose, polyethylene glycol, pullulan, simethicone, sodium bicarbonate, sodium carbonate, sodium chloride, sorbitol, starch, sucrose, trehalose, xylitol, and a combination of the foregoing. In one embodiment, Composition (A) comprises two or more pharmaceutical diluents. In another embodiment, Composition (A) comprises one pharmaceutical diluent. In a further embodiment, the pharmaceutical diluent is microcrystalline cellulose. Microcrystalline cellulose is a binder/diluent in oral tablet and capsule formulations and can be used in dry-granulation, wet-granulation, and direct-compression processes.

In some embodiments of the methods, Composition (A) comprises one or more pharmaceutical diluents in an amount from about 45 to about 80 wt %, from about 45 to about 75 wt %, from about 45 to about 70 wt %, from about 45 to about 65 wt %, from about 45 to about 60 wt %, or from about 45 to about 55 wt % of the total weight of the composition. In a further embodiment, the one or more pharmaceutical diluents comprises microcrystalline cellulose. In even a further embodiment, the compound of formula (I) is brensocatib, or a pharmaceutically acceptable salt thereof.

In some embodiments of the methods, Composition (A) comprises one or more pharmaceutical diluents in an amount from about 50 to about 85 wt %, from about 50 to about 75 wt %, from about 55 to about 85 wt %, from about 55 to about 70 wt %, from about 60 to about 85 wt %, from about 65 to about 85 wt %, from about 70 to about 85 wt %, or from about 75 to about 85 wt % of the total weight of the composition. In a further embodiment, the one or more pharmaceutical diluents is present at from about 55 to about 70 wt % of the total weight of the composition. In a further embodiment, the one or more pharmaceutical diluents comprises microcrystalline cellulose. In even a further embodiment, the compound of formula (I) is brensocatib, or a pharmaceutically acceptable salt thereof.

In some embodiments of the methods, Composition (A) comprises one or more pharmaceutical diluents in an amount of about 45 wt %, about 50 wt %, about 55 wt %, about 60 wt %, about 65 wt %, about 70 wt %, about 75 wt %, about 80 wt % or about 85 wt % of the total weight of the composition.

In some embodiments of the methods, the one or more pharmaceutical diluents in Composition (A) is microcrystalline cellulose. In other embodiments, the one or more pharmaceutical diluents comprises calcium carbonate, calcium phosphate, calcium sulfate, cellulose acetate, erythritol, ethylcellulose, fructose, inulin, isomalt, lactitol, magnesium carbonate, magnesium oxide, maltitol, maltodextrin, maltose, mannitol, polydextrose, polyethylene glycol, pullulan, simethicone, sodium bicarbonate, sodium carbonate, sodium chloride, sorbitol, starch, sucrose, trehalose and xylitol.

In the present disclosure, the terms “disintegrant” and “disintegrants” are intended to be interpreted in the context of pharmaceutical formulation science. Accordingly, a disintegrant in the Composition (A) may be, for example: alginic acid, calcium alginate, carboxymethylcellulose calcium, chitosan, croscarmellose sodium, crospovidone, glycine, guar gum, hydroxypropyl cellulose, low-substituted hydroxypropyl cellulose, magnesium aluminum silicate, methylcellulose, povidone, sodium alginate, sodium carboxymethylcellulose, sodium starch glycolate, starch, or a combination thereof.

In some embodiments of the methods, the one or more disintegrants in Composition (A) is sodium starch glycolate. In one embodiment, the amount of the disintegrants present in Composition (A) is between 2% and 8% of the total weight of the composition. In a further embodiment, the amount of the disintegrants is about 2 wt %, about 2.5 wt %, about 3 wt %, about 3.5 wt %, about 4 wt % or about 4.5 wt % of the total weight of the composition. The physical properties of sodium starch glycolate, and hence its effectiveness as a disintegrant, are affected by the degree of crosslinkage, extent of carboxymethylation, and purity.

In some embodiments of the methods, the one or more pharmaceutical disintegrants in Composition (A) comprises croscarmellose sodium.

In some embodiments of the methods, Composition (A) comprises one or more pharmaceutical disintegrants in an amount from about 2 to about 14 wt %, from about 2 to about 13 wt %, from about 2 to about 12 wt %, from about 2 to about 11 wt %, from about 2 to about 10 wt %, from about 2 to about 9 wt %, from about 2 to about 8 wt %, from about 2 to about 7 wt %, from about 2 to about 6 wt %, from about 2 to about 5 wt %, from about 3.5 to about 4.5 wt % of the total weight of the composition. In a further embodiment, the one or more pharmaceutical disintegrants is present at from about 3.5 to about 4.5 wt % of the total weight of the pharmaceutical composition. In a further embodiment, the one or more pharmaceutical disintegrants is sodium starch glycolate. In a further embodiment, the one or more pharmaceutical diluents comprises microcrystalline cellulose. In even a further embodiment, the compound of formula (I) is brensocatib, or a pharmaceutically acceptable salt thereof.

In the present disclosure, the terms “glidants” and “gliding agents” are intended to be interpreted in the context of pharmaceutical formulation science. Accordingly, a glidant in Composition (A) may be, for example: silicon dioxide, colloidal silicon dioxide, powdered cellulose, hydrophobic colloidal silica, magnesium oxide, magnesium silicate, magnesium trisilicate, sodium stearate and talc.

Accordingly, in some embodiments of the methods, the one or more pharmaceutical glidants in Composition (A) is selected from silicon dioxide, colloidal silicon dioxide, powdered cellulose, hydrophobic colloidal silica, magnesium oxide, magnesium silicate, magnesium trisilicate, sodium stearate, talc, or a combination of the foregoing. In one embodiment, the glidant is silicon dioxide. Its small particle size and large specific surface area give it desirable flow characteristics that are exploited to improve the flow properties of dry powders in a number of processes such as tableting and capsule filling. Typical silicon dioxide concentrations for use herein range from about 0.05 to about 1.0 wt %. Porous silica gel particles may also be used as a glidant, which may be an advantage for some formulations, with typical concentrations of 0.25-1%.

In some embodiments of the methods, Composition (A) comprises one or more pharmaceutical glidants in an amount from about 0.00 to about 1.75 wt %; from about 0.00 to about 1.50 wt %; from about 0.00 to about 1.25 wt %; from about 0.00 to about 1.00 wt %; from about 0.00 to about 0.75 wt %; from about 0.00 to about 0.50 wt %; from about 0.00 to about 0.25 wt %; from about 0.00 to about 0.20 wt % of the total weight of the composition. In a further embodiment, the one or more pharmaceutical glidants comprises silicon dioxide. In a further embodiment, the one or more pharmaceutical disintegrants is sodium starch glycolate. In a further embodiment, the one or more pharmaceutical diluents comprises microcrystalline cellulose. In even a further embodiment, the compound of formula (I) in Composition (A) is brensocatib, or a pharmaceutically acceptable salt thereof.

In some embodiments of the methods, Composition (A) comprises one or more pharmaceutical glidants in an amount from about 0.05 to about 2 wt %; from about 0.05 to about 1.75 wt %; from about 0.05 to about 1.50 wt %; from about 0.05 to about 1.25 wt %; from about 0.05 to about 1.00 wt %; from about 0.05 to about 0.75 wt %; from about 0.05 to about 0.50 wt %; from about 0.05 to about 0.25 wt %; or from about 0.05 to about 0.20 wt % of the total weight of the composition. In a further embodiment, the one or more pharmaceutical glidants is present at from about 0.05 to about 0.25 wt % of the total weight of the composition. In a further embodiment, the one or more pharmaceutical glidants comprises silicon dioxide. In a further embodiment, the one or more pharmaceutical disintegrants is sodium starch glycolate. In a further embodiment, the one or more pharmaceutical diluents comprises microcrystalline cellulose. In even a further embodiment, the compound of formula (I) in Composition (A) is brensocatib, or a pharmaceutically acceptable salt thereof.

In some embodiments of the methods, Composition (A) comprises one or more pharmaceutical glidants in an amount from about 0.05 to about 2 wt %; from about 0.10 to about 2 wt %; from about 0.2 to about 2 wt %; from about 0.3 to about 2 wt %; or from about 0.40 to about 2 wt % of the total weight of the composition. In a further embodiment, the one or more pharmaceutical glidants comprises silicon dioxide. In a further embodiment, the one or more pharmaceutical disintegrants is sodium starch glycolate. In a further embodiment, the one or more pharmaceutical diluents comprises microcrystalline cellulose. In even a further embodiment, the compound of formula (I) in Composition (A) is brensocatib, or a pharmaceutically acceptable salt thereof.

In the present disclosure, the terms “lubricant” and “lubricants”, as used herein, are intended to be interpreted in the context of pharmaceutical formulation science. Accordingly, a lubricant may be, for example calcium stearate, glyceryl behenate, glyceryl monostearate, glyceryl palmitostearate, a mixture of behenate esters of glycerine (e.g. a mixture of glyceryl bihenehate, tribehenin and glyceryl behenate), leucine, magnesium stearate, myristic acid, palmitic acid, poloxamer, polyethylene glycol, potassium benzoate, sodium benzoate, sodium lauryl sulfate, sodium stearate, sodium stearyl fumarate, stearic acid, talc, tribehenin and zinc stearate.

Accordingly, in some embodiments of the methods, the one or more pharmaceutical lubricants in Composition (A) are selected from the group consisting of calcium stearate, glyceryl behenate, glyceryl monostearate, glyceryl palmitostearate, a mixture of behenate esters of glycerine (e.g., a mixture of glyceryl bihenehate, tribehenin and glyceryl behenate), leucine, magnesium stearate, myristic acid, palmitic acid, poloxamer, polyethylene glycol, potassium benzoate, sodium benzoate, sodium lauryl sulfate, sodium stearate, sodium stearyl fumarate, stearic acid, talc, tribehenin and zinc stearate. In other embodiments, the one or more pharmaceutical lubricants are selected from the group consisting of calcium stearate, glyceryl behenate, glyceryl monostearate, glyceryl palmitostearate, a mixture of behenate esters of glycerine (e.g., a mixture of glyceryl bihenehate, tribehenin and glyceryl behenate), leucine, magnesium stearate, myristic acid, palmitic acid, poloxamer, polyethylene glycol, potassium benzoate, sodium benzoate, sodium lauryl sulfate, sodium stearate, stearic acid, talc, tribehenin and zinc stearate.

In some embodiments of the methods, Composition (A) comprises one or more pharmaceutical lubricants and the lubricant is not sodium stearyl fumarate. In a further embodiment, the compound of formula (I) in Composition (A) is brensocatib, or a pharmaceutically acceptable salt thereof.

In one embodiment of the methods, Composition (A) includes glycerol behenate as the lubricant.

In some embodiments of the methods, the one or more pharmaceutical lubricants in Composition (A) comprises glyceryl behenate, magnesium stearate, stearic acid, or a combination thereof.

In one embodiment of the methods, the lubricant in Composition (A) is glyceryl behenate, magnesium stearate, or a combination thereof.

In one embodiment of the methods, the one or more pharmaceutical lubricants in Composition (A) comprises sodium stearyl fumarate and/or one or more behenate esters of glycerine.

In some embodiments of the methods, Composition (A) comprises one or more pharmaceutical lubricants in an amount from about 1 wt % to about 9 wt %, from about 1 wt % to about 8 wt %, from about 1 wt % to about 7 wt %, from about 1 wt % to about 6 wt %, from about 1 wt % to about 5 wt %, from about 2 wt % to about 10 wt %, from about 2.5 wt % to about 10 wt %, from about 2 wt % to about 8 wt %, from about 2 wt % to about 7 wt %, from about 2 wt % to about 6 wt %, from about 2 wt % to about 5 wt %, from about 2 wt % to about 4.5 wt %, or from about 2.5 wt % to about 4.5 wt % of the total weight of the composition. In a further embodiment, the one or more pharmaceutical lubricants is present at from about 2.5 to about 4.5 wt % of the total weight of the composition. In a further embodiment, the one or more pharmaceutical lubricants in Composition (A) is glycerol behenate. In a further embodiment, the one or more pharmaceutical glidants in Composition (A) comprises silicon dioxide. In a further embodiment, the one or more pharmaceutical disintegrants in Composition (A) is sodium starch glycolate. In a further embodiment, the one or more pharmaceutical diluents in Composition (A) comprises microcrystalline cellulose. In even a further embodiment, the compound of formula (I) in Composition (A) is brensocatib, or a pharmaceutically acceptable salt thereof.

In one embodiment of the methods, the one or more pharmaceutical lubricants in Composition (A) consists of sodium stearyl fumarate and/or one or more behenate esters of glycerine or a mixture thereof.

In another embodiment of the methods, the one or more pharmaceutical lubricants in Composition (A) consists of sodium stearyl fumarate, glyceryl dibehenate, glyceryl behenate, tribehenin or any mixture thereof.

In one embodiment of the methods, the one or more pharmaceutical lubricants in Composition (A) comprises sodium stearyl fumarate. In another embodiment, the one or more pharmaceutical lubricants in Composition (A) consists of sodium stearyl fumarate.

In one embodiment of the methods, the one or more pharmaceutical lubricants in Composition (A) comprises one or more behenate esters of glycerine (i.e., one or more of glyceryl dibehenate, tribehenin and glyceryl behenate).

In one embodiment of the methods, the compression aid in Composition (A) is dicalcium phosphate dihydrate (also known as dibasic calcium phosphate dihydrate) (DCPD). DCPD is used in tablet formulations both as an excipient and as a source of calcium and phosphorus in nutritional supplements.

In one embodiment of the methods, Composition (A) comprises the compression aid, e.g., DCPD, in an amount from about 10 to about 30 wt %, including about 16 wt %, about 17 wt %, about 18 wt %, about 19 wt %, about 20 wt %, about 21 wt %, about 22 wt %, about 23 wt %, or about 24 wt % of the total weight of the composition. In a further embodiment, the compression aid is present at about 20 wt % of the total weight of the composition.

In one embodiment of the methods, Composition (A) comprises the compression aid, e.g., DCPD, in an amount from about 10 to about 25 wt %, from about 10 to about 20 wt %, from about 10 to about 15 wt %, from about 15 to about 25 wt %, or from about 20 to about 25 wt %, or from about 18 to about 22 wt % of the total weight of the composition. In a further embodiment, the compression aid is present at from about 18 to about 22 wt % of the total weight of the composition. In a further embodiment, the compression aid is DCPD. In a further embodiment, the one or more pharmaceutical lubricants in Composition (A) is glycerol behenate. In a further embodiment, the one or more pharmaceutical glidants in Composition (A) comprises silicon dioxide. In a further embodiment, the one or more pharmaceutical disintegrants in Composition (A) is sodium starch glycolate. In a further embodiment, the one or more pharmaceutical diluents in Composition (A) comprises microcrystalline cellulose. In even a further embodiment, the compound of formula (I) in the exemplary composition is brensocatib, or a pharmaceutically acceptable salt thereof.

In one embodiment of the methods, the pharmaceutical composition administered to the patient is Composition (B) comprising:
(a) from about 1 to about 30 wt % of the compound of formula (I), or a pharmaceutically acceptable salt thereof;
(b) from about 55 to about 75 wt % of a pharmaceutical diluent;
(c) from about 15 to about 25 wt % of a compression aid;
(d) from about 3 to about 5 wt % of a pharmaceutical disintegrant;
(e) from about 0.00 to about 1 wt % of a pharmaceutical glidant; and
(f) from about 2 to about 6 wt % of a pharmaceutical lubricant;
wherein the components add up to 100 wt %.

In some embodiments of the methods where Composition (B) is administered to the patient, the identity of the pharmaceutical diluent, compression aid, pharmaceutical disintegrant, pharmaceutical glidant, and pharmaceutical lubricant in the composition may be one of those described above for Composition (A). In other embodiments, the amount of the pharmaceutical diluent, compression aid, pharmaceutical disintegrant, pharmaceutical glidant, and pharmaceutical lubricant in Composition (B) may also be one of those described above for Composition (A), as long as the amount is within the corresponding broader range recited above for Composition (B).

The pharmaceutical compositions disclosed herein, including Compositions (A) and (B), may be in a solid dosage form suitable for oral administration to a human being. For example, the pharmaceutical composition is a pharmaceutical tablet. Pharmaceutical tablets may be prepared using methods known to those skilled in the art including, for example, dry mixing/direct compression process as described in International Application Publication No. WO 2019/166626. In some embodiments, the pharmaceutical tablet comprises a tablet core wherein the tablet core comprises the pharmaceutical composition as disclosed herein and wherein the tablet core has a coating. In some embodiments, the coating is a film coating. The film coating may be applied using conventional methods known to those skilled in the art. A functional coating can be used to provide protection against, for example, moisture ingress or degradation by light. Additionally, a functional coating may be used to modify or control the release of the compound of formula (I), e.g., brensocatib, from the composition. The coating may comprise, for example, about 0.2 to about 10 wt % of the total weight of the pharmaceutical composition, e.g., from about 0.2 to about 4 wt %, from about 0.2 to about 3 wt %, from about 1 to about 6 wt %, or from about 2 to about 5 wt % of the total weight of the pharmaceutical composition.

### DPP1-Mediated Conditions

In some embodiments, the DPP1-mediated condition amenable to the treatment methods provided herein is an obstructive disease of the airways. Non-limiting examples of an obstructive disease of the airways include asthma, including bronchial, allergic, intrinsic, extrinsic, exercise-induced, drug-induced (including aspirin and NSAID-induced) and dust-induced asthma, both intermittent and persistent and of all severities, and other causes of airway hyper responsiveness; chronic obstructive pulmonary disease (COPD); bronchitis, including infectious and eosinophilic bronchitis; emphysema; bronchiectasis; cystic fibrosis; sarcoidosis; alpha-1 antitrypsin deficiency; farmer's lung and related diseases; hypersensitivity pneumonitis; lung fibrosis, including cryptogenic fibrosing alveolitis, idiopathic interstitial pneumonias, fibrosis complicating anti-neoplastic therapy and chronic infection, including tuberculosis and aspergillosis and other fungal infections; complications of lung transplantation; vasculitic and thrombotic disorders of the lung vasculature, and pulmonary hypertension; antitussive activity including treatment of chronic cough associated with inflammatory and secretory conditions of the airways, and iatrogenic cough; acute and chronic rhinitis including rhinitis medicamentosa, and vasomotor rhinitis; perennial and seasonal allergic rhinitis including rhinitis nervosa (hay fever); nasal polyposis; obstructive diseases of the airways due to acute viral infection including the common cold, and infection due to respiratory syncytial virus, influenza, coronavirus (including SARS) and adenovirus, acute lung injury, and acute respiratory distress syndrome (ARDS), as well as exacerbations of each of the foregoing respiratory tract disease states.

In one embodiment, the DPP1-mediated condition treated by the methods is asthma (such as bronchial, allergic, intrinsic, extrinsic or dust asthma, particularly chronic or inveterate asthma (for example late asthma or airways hyper-responsiveness)).

In one embodiment, the DPP1-mediated condition treated by the methods is chronic obstructive pulmonary disease (COPD).

In one embodiment, the DPP1-mediated condition treated by the methods is allergic rhinitis.

In one embodiment, the DPP1-mediated condition treated by the methods is alpha-1 antitrypsin deficiency.

In one embodiment, the DPP1-mediated condition treated by the methods is acute respiratory distress syndrome (ARDS).

In one embodiment, the DPP1-mediated condition treated by the methods is bronchiectasis. The bronchiectasis may be in a patient with cystic fibrosis, or in a patient that does not have cystic fibrosis (sometimes referred to as “bronchiectasis unrelated to cystic fibrosis” or “non-cystic fibrosis (CF) bronchiectasis”). Methods of treating bronchiectasis using a compound of formula (I) are described in U.S. Application Publication No. 2018/0028541, which is incorporated by reference herein in its entirety for all purposes.

Bronchiectasis is considered a pathological endpoint that results from many disease processes and is a persistent or progressive condition characterized by dilated thick-walled bronchi. The symptoms vary from intermittent episodes of expectoration and infection localized to the region of the lung that is affected to persistent daily expectoration often of large volumes of purulent sputum. Bronchiectasis may be associated with other non-specific respiratory symptoms. The underlying pathological process of bronchiectasis, without wishing to be bound by theory, has been reported as damage to the airways which results from an event or series of events where inflammation is central to the process (Guideline for non-CF Bronchiectasis, Thorax, July 2010, V. 65(Suppl 1), incorporated by reference herein in its entirety for all purposes). Non-CF bronchiectasis has been reported to be caused by or associated with numerous etiologies ranging from genetic illness to retained airway foreign body, and has been reported to be present in patients with systemic disease, common respiratory diseases such as chronic obstructive pulmonary disease (COPD) as well as uncommon diseases such as sarcoidosis (Chang and Bilton (2008). Thorax 63, pp. 269-276, incorporated by reference herein in its entirety for all purposes).

In one embodiment, the DPP1-mediated condition treated by the methods is an antineutrophil cytoplasmic autoantibody (ANCA) associated vasculitis, including, but not limited to, granulomatosis with polyangiitis (GPA) or microscopic polyangiitis (MPA)). Methods of treating ANCA associated vasculitis (e.g., GPA or MPA) using a compound of formula (I) are described in U.S. Application Publication No. 2019/0247400, which is incorporated by reference herein in its entirety for all purposes.

In one embodiment, the DPP1-mediated condition treated by the methods is cystic fibrosis. Cystic fibrosis (CF) is caused by abnormalities in the CF transmembrane conductance regulator protein, causing chronic lung infections (particularly with Pseudomonas aeruginosa) and excessive inflammation, and leading to bronchiectasis, declining lung function, respiratory insufficiency and quality of life. The inflammatory process is dominated by neutrophils that produce NE, as well as other destructive NSPs including CatG and PR3, that directly act upon extracellular matrix proteins and play a role in the host response to inflammation and infection (Dittrich et al., Eur Respir J. 2018; 51(3)). Without wishing to be bound by theory, it is thought that the compounds of formula (I), which are reversible inhibitors of DPP1, administered via the methods provided herein have beneficial effects via effective inhibition of the activation of NSPs and decreasing inflammation, which in turn leads to a decrease in pulmonary exacerbations, a decrease in the rate of pulmonary exacerbations, and/or an improvement in lung function (e.g., forced expiratory volume in 1 second [FEV₁]) in CF patients.

In some embodiments, the DPP1-mediated condition amenable to the treatment methods provided herein is cancer, including a primary solid tumor, a liquid tumor, or a metastatic cancer. In one embodiment, the DPP1 is expressed by cancerous cells, neutrophils, macrophages, monocytes, or mast cells of a cancer patient.

NSPs, including neutrophil elastase (NE), proteinase 3 (PR3), cathepsin G (CatG), and neutrophil serine protease 4 (NSP4), activated by DPP1 can mediate tumor initiation, tumor progression and/or tumor metastasis. Moreover, neutrophils play an important role in stages of metastasis, such as, intravascular dissemination, extravasation, and metastatic growth. Neutrophils can aid cancer cell adhesion to the endothelium in metastatic sites with their surface expression of selectins and integrins. Neutrophil-derived IL-1β can promote tumor cell extravasation. Furthermore, neutrophil extracellular traps (NETs) can induce invasive and migratory behaviors of tumor cells. NETs can also result in the degradation of thrombospondin-1, which in turn facilitates metastatic cancer growth. Without wishing to be bound by a theory, it is thought that the inhibition of DPP1 function by the compounds of formula (I) can result in the inhibition of NSPs and/or the pro-cancerous functions of neutrophils, and therefore, inhibition of the development, growth and the progression of a variety of cancers, and cancer metastasis at various stages (such as, intravascular dissemination, extravasation).

In one embodiment, the DPP1-mediated condition treated by the methods is cancer comprising a primary solid tumor. In some embodiments, the cancer is selected from the group consisting of breast cancer, bladder cancer, lung cancer, brain cancer, ovarian cancer, pancreatic cancer, colorectal cancer, prostate cancer, liver cancer, hepatocellular carcinoma, kidney cancer, stomach cancer, skin cancer, fibroid cancer, lymphoma, virus-induced cancer, oropharyngeal cancer, testicular cancer, thymus cancer, thyroid cancer, melanoma, and bone cancer.

In one embodiment, the cancer is bladder cancer.

In one embodiment, the cancer is lung cancer.

In one embodiment, the cancer is brain cancer. In some embodiments, the brain cancer is astrocytoma, anaplastic astrocytoma, glioblastoma multiforme, oligodendroglioma, ependymoma, meningioma, schwannoma, or medulloblastoma. In some embodiments, the brain cancer is astrocytoma. In some embodiments, the brain cancer is anaplastic astrocytoma. In some embodiments, the brain cancer is glioblastoma multiforme. In some embodiments, the brain cancer is oligodendroglioma. In some embodiments, the brain cancer is ependymoma. In some embodiments, the brain cancer is meningioma. In some embodiments, the brain cancer is schwannoma. In some embodiments, the brain cancer is medulloblastoma.

In one embodiment, the cancer is ovarian cancer.

In one embodiment, the cancer is pancreatic cancer.

In one embodiment, the cancer is colorectal cancer.

In one embodiment, the cancer is prostate cancer.

In one embodiment, the cancer is liver cancer.

In one embodiment, the cancer is hepatocellular carcinoma.

In one embodiment, the cancer is kidney cancer.

In one embodiment, the cancer is stomach cancer.

In one embodiment, the cancer is skin cancer.

In one embodiment, the cancer is fibroid cancer. In a further embodiment, the fibroid cancer is leiomyosarcoma.

In one embodiment, the cancer is lymphoma. In some embodiments, the lymphoma is Hodgkin's lymphoma, non-Hodgkin's lymphoma, diffuse large B-cell lymphoma, B-cell immunoblastic lymphoma, Natural Killer cell lymphoma, T-cell lymphoma, Burkitt lymphoma or Kaposi's Sarcoma. In some embodiments, the lymphoma is Hodgkin's lymphoma. In some embodiments, the lymphoma is non-Hodgkin's lymphoma. In some embodiments, the lymphoma is diffuse large B-cell lymphoma. In some embodiments, the lymphoma is B-cell immunoblastic lymphoma. In some embodiments, the lymphoma is Natural Killer cell lymphoma. In some embodiments, the lymphoma is T-cell lymphoma. In some embodiments, the lymphoma is Burkitt lymphoma. In some embodiments, the lymphoma is Kaposi's Sarcoma.

In one embodiment, the cancer is virus-induced cancer.

In one embodiment, the cancer is oropharyngeal cancer.

In one embodiment, the cancer is testicular cancer.

In one embodiment, the cancer is thymus cancer.

In one embodiment, the cancer is thyroid cancer.

In one embodiment, the cancer is melanoma.

In one embodiment, the cancer is bone cancer.

In one embodiment, the cancer is breast cancer. In some embodiments, the breast cancer comprises ductal carcinoma, lobular carcinoma, medullary carcinoma, colloid carcinoma, tubular carcinoma, or inflammatory breast cancer. In some embodiments, the breast cancer comprises ductal carcinoma. In some embodiments, the breast cancer comprises lobular carcinoma. In some embodiments, the breast cancer comprises medullary carcinoma. In some embodiments, the breast cancer comprises colloid carcinoma. In some embodiments, the breast cancer comprises tubular carcinoma. In some embodiments, the breast cancer comprises inflammatory breast cancer.

In some embodiments, the breast cancer is triple-negative breast cancer. In some embodiments, the breast cancer does not respond to hormonal therapy or therapeutics that target the HER2 protein receptors.

In one embodiment, the DPP1-mediated condition treated by the methods is cancer comprising liquid tumor. In some embodiments, the liquid tumor is selected from the group consisting of acute myeloid leukemia (AML), acute lymphoblastic leukemia, acute lymphocytic leukemia, acute promyelocytic leukemia, chronic myeloid leukemia, hairy cell leukemia, myeloproliferative disorders, Natural Killer cell leukemia, blastic plasmacytoid dendritic cell neoplasm, chronic myelogenous leukemia (CML), mastocytosis, chronic lymphocytic leukemia (CLL), multiple myeloma (MM), and myelodysplastic syndrome (MDS). In some embodiments, the liquid tumor is acute myeloid leukemia (AML). In some embodiments, the liquid tumor is acute lymphoblastic leukemia. In some embodiments, the liquid tumor is acute lymphocytic leukemia. In some embodiments, the liquid tumor is acute promyelocytic leukemia. In some embodiments, the liquid tumor is chronic myeloid leukemia. In some embodiments, the liquid tumor is hairy cell leukemia. In some embodiments, the liquid tumor is a myeloproliferative disorder. In some embodiments, the liquid tumor is Natural Killer cell leukemia. In some embodiments, the liquid tumor is blastic plasmacytoid dendritic cell neoplasm. In some embodiments, the liquid tumor is chronic myelogenous leukemia (CML). In some embodiments, the liquid tumor is mastocytosis. In some embodiments, the liquid tumor is chronic lymphocytic leukemia (CLL). In some embodiments, the liquid tumor is multiple myeloma (MM). In some embodiments, the liquid tumor is myelodysplastic syndrome (MDS).

In one embodiment, the DPP1-mediated condition treated by the methods is a pediatric cancer. In some embodiments, the pediatric cancer is neuroblastoma, Wilms tumor, rhabdomyosarcoma, retinoblastoma, osteosarcoma or Ewing sarcoma. In some embodiments, the pediatric cancer is neuroblastoma. In some embodiments, the pediatric cancer is Wilms tumor. In some embodiments, the pediatric cancer is rhabdomyosarcoma. In some embodiments, the pediatric cancer is retinoblastoma. In some embodiments, the pediatric cancer is osteosarcoma. In some embodiments, the pediatric cancer is Ewing sarcoma.

In some embodiments, the DPP1-mediated condition treated by the methods is metastatic cancer. In some embodiments, the patient is at a risk for developing metastatic cancer. In some embodiments, the metastatic cancer comprises metastatic breast cancer. In a further embodiment, the metastatic breast cancer comprises metastasis of breast cancer to the lung, brain, bone, pancreas, lymph nodes, and/or liver. In still a further embodiment, the metastatic breast cancer comprises metastasis of breast cancer to the lung. In other embodiments, the metastatic cancer comprises metastasis of bone cancer to the lung. In other embodiments, the metastatic cancer comprises metastasis of colorectal cancer to the peritoneum, the pancreas, the stomach, the lung, the liver, the kidney, and/or the spleen. In other embodiments, the metastatic cancer comprises metastasis of stomach cancer to the mesentery, the spleen, the pancreas, the lung, the liver, the adrenal gland, and/or the ovary. In other embodiments, the metastatic cancer comprises metastasis of leukemia to the lymph nodes, the lung, the liver, the hind limb, the brain, the kidney, and/or the spleen. In other embodiments, the metastatic cancer comprises metastasis of liver cancer to the intestine, the spleen, the pancreas, the stomach, the lung, and/or the kidney. In other embodiments, the metastatic cancer comprises metastasis of lymphoma to the kidney, the ovary, the liver, the bladder, and/or the spleen.

In other embodiments, the metastatic cancer comprises metastasis of hematopoietic cancer to the intestine, the lung, the liver, the spleen, the kidney, and/or the stomach. In other embodiments, the metastatic cancer comprises metastasis of melanoma to lymph nodes and/or the lung. In other embodiments, the metastatic cancer comprises metastasis of pancreatic cancer to the mesentery, the ovary, the kidney, the spleen, the lymph nodes, the stomach, and/or the liver. In other embodiments, the metastatic cancer comprises metastasis of prostate cancer to the lung, the pancreas, the kidney, the spleen, the intestine, the liver, the bone, and/or the lymph nodes. In other embodiments, the metastatic cancer comprises metastasis of ovarian cancer to the diaphragm, the liver, the intestine, the stomach, the lung, the pancreas, the spleen, the kidney, the lymph nodes, and/or the uterus. In other embodiments, the metastatic cancer comprises metastasis of myeloma to the bone.

In other embodiments, the metastatic cancer comprises metastasis of lung cancer to the bone, the brain, the lymph nodes, the liver, the ovary, and/or the intestine. In other embodiments, the metastatic cancer comprises metastasis of kidney cancer to the liver, the lung, the pancreas, the stomach, the brain, and/or the spleen. In other embodiments, the metastatic cancer comprises metastasis of bladder cancer to the bone, the liver and/or the lung. In other embodiments, the metastatic cancer comprises metastasis of thyroid cancer to the bone, the liver and/or the lung.

### EXAMPLES

The present invention is further illustrated by reference to the following Examples. However, it should be noted that these Examples, like the embodiments described above, are illustrative and are not to be construed as restricting the scope of the invention in any way.

Examples 1-8 below investigate conditions and procedures for improved extraction and activity assays of NE, PR3, and CatG from patient samples comprising white blood cells (WBCs).

### Materials

Table 1A shows the compositions of various lysis buffers tested for extracting NE PR3, and CatG from human WBC samples, and of the reagents and buffers for measuring the enzymatic activity of NE, PR3, and CatG in wash fractions as well as cell lysates containing extracted NE, PR3 and CatG, respectively.

TABLE 1A
Lysis buffers for extracting NE, PR3, and CatG and reagents
and buffers for NE, PR3, and CatG activity assays

| | Reference Name | Formulation |
|---|---|---|
| Lysis | 0.02% Triton ® X-100¹ | 1xPBS + 150 nM NaCl + 0.02% (v/v) |
| Buffers | Lysis Buffer | Triton ® X-100¹ |
| | 1% Triton ® X-100¹ | 1xPBS + 150 nM NaCl + 1% (v/v) |
| | Lysis Buffer | Triton ® X-100¹ |
| | 10% Triton ® X-100¹ | 1xPBS + 150 nM NaCl + 10% (v/v) |
| | Lysis Buffer | Triton ® X-100¹ |
| | Abcam Lysis Buffer | 1xAbcam mammalian cell lysis buffer |
| | | (Cat. No. Ab179835) |
| | NP-40 Lysis Buffer | 50 mM HEPES buffer + 0.75M NaCl + 0.05% |
| | | (v/v) Nonidet ® P-40 (IUPAC name: |
| | | octylphenoxypolyethoxyethanol) |
| Assay | Assay Buffer | 100 mM Tris, 100 mM NaCl in H₂O, pH 7.5 |
| Reagents & | Enzyme Buffer | 0.05% (v/v) Triton ® X-100¹ in Assay Buffer |
| Buffers | NE Substrate | 2M NaCl in Assay Buffer |
| | Diluent | |
| | NE Substrate | Methoxysuccinyl-ala-ala-pro-val-AMC² |
| | PR3 Substrate | Abz³-VADCADQ-Lys(DNP) |
| | Human NE Stock | Elastase from Human Leukocytes (100 ng/μL in |
| | Protein | 50% glycerol-PBS v/v) |
| | Human PR3 Stock | PR3 from Human Neutrophils (50 ng/μL in 50% |
| | Protein | glycerol-PBS v/v) |
| | Human CatG Stock | CatG from Human Leukocytes (100 ng/μL in 50% |
| | Protein | glycerol-PBS v/v) |

¹The IUPAC name of Triton ® X-100 is 2-[4-(2,4,4-trimethylpentan-2-yl)phenoxy]ethanol.
²AMC = 7-amino 4-methyl coumarin
³Abz = 2-Aminobenzoyl or Anthraniloyl

### Methods

### 1. Sample Processing

Prior to performing NE PR3, and CatG extractions, whole blood samples were processed into a white blood cell (WBC) pellet by lysing 2 mL whole blood samples with 40 mL 1× red blood cell (RBC) lysis buffer (Abcam, Cat No. Ab204733), inverting 5 times, and incubating at room temperature for 20 minutes. Samples were then spun down at 400 g for 5 minutes and 4° C., followed by carefully decanting the liquid without disrupting the WBC pellet. WBC pellets intended for NE and PR3 extraction were frozen and stored at −80° C. To ready the WBC pellets for NE and PR3 extraction, the pellets were thawed and subjected to pellet lysis with a lysis buffer. In certain studies where NP-40 Lysis Buffer was used for NE and PR3 extraction, the thawed pellets were washed prior to pellet lysis (i.e., pre-lysis wash, described below). For WBC pellets intended for CatG extraction, some of them were likewise frozen and stored at −80° C., and later thawed and washed prior to pellet lysis with NP-40 Lysis Buffer. Some of the WBC pellets intended for CatG extraction were subjected to post-RBC lysis wash before freezing and storage at −80° C. Namely, WBC pellets obtained following RBC lysis were washed with either 40 mL Assay Buffer or 40 mL 0.9% saline, inverted 5 times to mix, and centrifuged (400×g for 5 minutes, 4° C.), followed by carefully decanting the liquid without disrupting the WBC pellets. The washed pellets were then frozen and stored at −80° C. To extract CatG, the frozen post-RBC lysis washed WBC pellets were thawed and subjected to pellet lysis with NP-40 Lysis Buffer or 0.02% Triton X-100 Lysis Buffer, without undergoing pre-lysis wash even when NP-40 lysis buffer was used. See Table 7 for a summary of cell pellet processing and lysis conditions in Example 8 related to CatG extraction from WBC pellets. For experiments where different lysis conditions were compared, multiple WBC pellets were generated from each donor whole blood sample.

### 2. Pre-Lysis Wash of WBC Pellets

In some experiments where NP-40 Lysis Buffer was used to extract NE, PR3, and CatG from a WBC pellet, prior to pellet lysis with NP-40 Lysis Buffer, a frozen WBC pellet was thawed at room temperature and washed with 1 mL ice-cold Assay Buffer by gently pipetting the mixture of the pellet and the Assay Buffer. The mixture was centrifuged at 16,000 g for 3 minutes at 4° C. to obtain a washed pellet and a supernatant (i.e., the wash fraction). The washed pellet was subjected to lysis to extract NE, PR3, and CatG. The wash fraction was collected and transferred to an empty microfuge tube for NE, PR3, and CatG activity assay. In order to account for varying volumes of residual RBC lysis buffer that might be present in the WBC pellet sample prior to washing, the microcentrifuge tube was weighed before and after the wash fraction was transferred, and the weight difference was calculated. The volume of the wash fraction was obtained based on the weight difference, which was converted to volume using the density of 1 g/mL (the density of water). As noted above, pre-lysis wash was not performed with WBC pellets that had previously been washed post-RBC lysis for CatG extraction.

### 3. WBC Pellet Lysis

WBC pellet lysis was performed by adding 1 mL lysis buffer to an unwashed WBC pellet that had previously been frozen and thawed at room temperature, or a pre-lysis washed WBC pellet, or a post-RBC lysis washed WBC pellet, followed by agitation with pipetting. Debris was pelleted via centrifugation at 16,000 g for 3-10 minutes at 4° C. and the supernatant (referred to as “1° cell lysate”) was collected and stored at −80° C. for NE, PR3, and CatG activity assays. In multi-extraction experiments where a WBC pellet was subjected to a multi-step repeated pellet lysis process, the debris was subjected to additional steps (rounds or cycles) of lysis. During each additional lysis step, the debris from the previous lysis step was lysed by the same or a different lysis buffer with agitation. Thereafter, the remaining debris was likewise pelleted via centrifugation and subjected to the next lysis step, while the supernatant was collected as an additional cell lysate, likewise referred to as 2°, 3°, 4°, 5° cell lysate, etc., with the number in the nomenclature matching the number of the originating lysis step. The amount of mechanical agitation applied during a lysis step, and the number of lysis steps in the repeated pellet lysis process were varied to assess their effects on NE and PR3 recovery. For CatG extraction and activity determination, each WBC pellet was subjected to a total of three lysis cycles, with 20 times of mechanical pipetting applied during each lysis cycle, and with the resultant 1°, 2°, and 3° cell lysates pooled (see Table 7 in Example 8 for a summary of cell pellet processing and lysis conditions). To minimize variation as well as reduce the total extraction time, multiple WBC pellets from each donor were processed at the same time by using a multichannel pipette and under substantially the same conditions, e.g., the same duration of lysis and same amount of mechanical agitation.

### 4. NE, PR3, and CatG Assays

The activity of NE, PR3, and CatG was measured in one of two ways: (1) ELISA-based assays, i.e., ProteaseTag® Active NE Immunoassay, ProteaseTag® Active PR3 Immunoassay, and ProteaseTag® Active CatG Immunoassay from ProAxsis (Belfast, Northern Ireland), or (2) enzymatic kinetic assays, each of which uses an exogenous peptide substrate specific to NE, PR3, or CatG. For comparison, CatG activity was also measured by using SensoLyte® Rh110 Cathepsin G Assay Kit (AnaSpec, Fremont, CA), which is fluorometric enzymatic assay that detects and quantifies CatG activity in biological samples.

The ELISA-based assays, each of which detects and quantifies active NE, PR3, or CatG, but not the latent or inhibitor bound counterpart, were performed according to the manufacturer's instructions.

In the NE kinetic assay, an exogenous peptide substrate with high specificity for NE (shown in Table 1A) was cleaved by NE present in the sample, generating a fluorophore reaction product 7-amino 4-methyl coumarin (AMC). The initial rate of this reaction, which is proportional to the amount of active NE in the sample, was measured in relative fluorescence units (RFU) and converted to the concentration of active NE in the sample. Specifically, standards were created via serial dilution of Human NE Stock Protein (Sigma, Cat. No E8140-1UN, see Table 1A) in a standard diluent, which matched the ratio of lysis buffer to Enzyme Buffer (sample diluent) of the sample dilutions on a multi-well plate. Sample dilutions were created on the plate using Enzyme Buffer as the diluent. A 1:25 dilution of either DMSO or NE inhibitor (Abcam, Cat. No ab142154, final concentration 80 μM) in Assay Buffer was added to all wells and allowed to incubate at 37° C. for 15 minutes. Addition of DMSO was used as a placeholder in case future testing required addition of inhibitors for subtraction of nonspecific activity-induced cleavage. Following incubation, NE Substrate (Methoxysuccinyl-ala-ala-pro-val-AMC, Sigma, Cat No M9771, final concentration 100 μM) diluted in NE Substrate Diluent was added to the sample and control wells in that order, and briefly mixed via pipetting 2-3 times. The plate was immediately read at 350/450 nm (Excitation/Emission) post substrate addition in kinetic mode every 5 minutes for up to 3 hours (minimum 30 minutes) at 37° C. on a BioTek Gen5 Plate Reader. The raw data of the readings were exported into an Excel file for data analysis described below.

Except for using an exogenous peptide substrate specific for PR3 instead of NE, the PR3 kinetic assay is based on the same principle as the NE kinetic assay described above. Cleavage of PR3 Substrate by PR3 present in the sample generated the fluorophore reaction product 2-Aminobenzoyl or Anthraniloyl (Abz). The initial rate of this reaction, which is proportional to the amount of active PR3 in the sample, was measured in RFU and converted to the concentration of active PR3 in the sample. Specifically, standards were created via serial dilution of Human PR3 Stock Protein (Sigma, Cat. No SRP6309-25UG, see Table 1A) in a standard diluent which matched the ratio of lysis buffer to Enzyme Buffer (sample diluent) of the sample dilutions on a multi-well plate. Sample dilutions were created on the plate using Enzyme Buffer as the diluent. A 1:20 dilution of either DMSO or PR3 inhibitor (Abcam, Cat. No ab146184, final concentration 500 μM) in Assay Buffer was added to all wells and allowed to incubate at 37° C. for 15 minutes. Addition of DMSO was used as a placeholder in case future testing required addition of inhibitors for subtraction of nonspecific activity-induced cleavage. Following incubation, PR3 Substrate (Abz-VADCADQ-Lys(DNP), final concentration 100 μM) diluted in Assay Buffer was added to the sample and control wells in that order, and briefly mixed via pipetting 2-3 times. The plate was immediately read at 340/430 nm (Excitation/Emission) post substrate addition in kinetic mode every 5 minutes for up to 3 hours (minimum 30 minutes) at 37° C. on a BioTek Gen5 Plate Reader. The raw data of the readings were exported into an Excel file for data analysis described below.

CatG activity was measured in an enzymatic assay using an exogenous peptide substrate. Cleavage of the substrate generated the chromophore or fluorophore reaction product, p-Nitroaniline (pNA), 6-Carboxytetramethylrhodamine (6-TAMRA) or 7-Methoxycoumarin-4-acetic acid (MCA). The initial rate of this reaction is proportional to the amount of active CatG in a sample and was measured in absorbance (ABS) or fluorescence (RFU), depending on the substrate, and converted to the concentration of active CatG in the sample. Specifically, standards were created via serial dilution of stock human CatG protein (Sigma, Cat. No C4428-.25UN) in a standard diluent which matched the ratio of lysis buffer to Enzyme Buffer (sample diluent) of the sample dilutions on plate. Sample dilutions were created on the plate using Enzyme Buffer as the diluent and/or run neat. A 1:10 dilution of either DMSO or CatG inhibitor (Cayman Chemical, Cat. No 14928, final concentration 200 μM) in Assay Buffer was added to all wells and allowed to incubate at 37° C. for 15 minutes. Addition of DMSO was used as a placeholder in case future testing required addition of inhibitors for subtraction of nonspecific activity cleavage. Following incubation, CatG substrate diluted in Assay Buffer was added to the sample and control wells in that order, and briefly mixed via pipetting 2-3 times (see Table 1B for list of tested substrates). The plate was immediately read at appropriate wavelength according to Table 1B post substrate addition in kinetic mode every 5 minutes for 1.5 hours at 37° C. on a BioTek's Synergy Neo or BioTek's Synergy HIM plate reader with Gen5 Software. The experiment was saved, and the raw data was exported into an Excel file for data analysis (see Data Analysis methods below).

TABLE 1B
CatG Substrates

| | | | Catalog | Final Assay | | Absorbance or | |
|---|---|---|---|---|---|---|---|
| Substrate | Type | Vendor | Number | Concentration | | Excitation/Emission | |
| Suc-AAPF- | Colorimetric | Sigma | S7388 | 400 | μM | 405 | nm |
| pNA | | | | | | | |
| [5-FAM]- | Fluorometric | Discovery | crb1100420 | 40, 20, 8, 4 | μM | 492/514 | nm |
| Glu-Pro- | | Peptides | | | | | |
| Phe-Trp- | | | | | | | |
| Glu-Asp- | | | | | | | |
| Gln-Lys[(6- | | | | | | | |
| TAMRA)]- | | | | | | | |
| NH₂ | | | | | | | |
| Mca-FVT- | Fluorometric | Millipore | 219474 | 40, 10 | μM | 325/400 | nm |
| Gnf-SW- | | Sigma | | | | | |
| Anb-NH₂ | | | | | | | |

### 5. Data Analysis

To analyze the data from the ELISA-based assays, standard curves were created using the standard absorbance values and their respective known concentrations. Multiple standard curves were created if multiple standard diluents were used in the assay. The unknown sample concentrations were then calculated using the second-degree polynomial line of best fit formula from the appropriate standard curve (when available). Sample concentrations were corrected for dilution and duplicates were averaged.

To analyze the data from the NE and PR3 kinetic assays, two methods were used and the results were compared to ensure consistency. Specifically, the linear portions of the kinetic slopes were either (1) visually determined and calculated using Excel's slope formula, or (2) automatically determined and calculated using an internally developed macro Excel program. Standard curves were created using the standard slope values and their respective known concentrations. Multiple standard curves were created if multiple standard diluents were used in the assay. The unknown sample concentrations were then calculated using the second-degree polynomial line of best fit formula from the appropriate standard curves (when available). Sample concentrations were corrected for dilution and duplicates were averaged.

To analyze the data from the CatG kinetic assays, readings from BioTek's Synergy Neo and H1 plate reader with Gen5 software and Imager Software were directly exported into an Excel file containing the raw data. These raw data readings from each plate were then copied over to a second Excel file for data analysis. The linear portion of the kinetic slopes were visually determined and calculated using Excel's slope formula. Standard curves were created using the standard slope values and their respective known concentrations. Multiple standard curves were created if multiple standard diluents were used in the assay. The unknown sample concentrations were then calculated using the second-degree polynomial line of best fit formula from the appropriate standard curves (when available). Sample concentrations were corrected for dilution and duplicates were averaged.

Unless otherwise noted, all of the WBC sample concentrations of active NE, PR3, and CatG presented in the examples are normalized to the volume of whole blood from which a WBC sample was derived, and are expressed as mass (e.g., in ng or μg) of active NE, PR3, or CatG per mL of whole blood.

### 6. Statistical Analysis

Statistical analysis of WBC pellet extraction results was performed using Dunnett's multiple comparison test. The alpha value was set at 0.05.

### Example 1—Lysis Buffer Screening for Extraction of NE and PR3 from Human WBC Samples

Neutrophil serine proteases (NSPs), such as NE and PR3, are encapsulated inside the azurophilic granules of neutrophils and can be released to provide a rapid immune response. A portion of NSPs may also be present generally within neutrophils. In order to extract these enzymes for quantitation, both the cell and the granules must be lysed. Traditional methods of lysis include physical disruption (e.g., agitation) and chemical means (e.g., by using a detergent) to break open the cell membrane and expose the NSPs. The suitability of different detergents at different concentrations for the extraction of NSPs is unpredictable, since the choice of detergent and its concentration may not only affect the recovery of the NSP, but also interfere with a downstream NSP quantification or activity assay. To compare NE and PR3 recovery under different detergent conditions, 0.02% Triton® X-100 Lysis Buffer, 1% Triton® X-100 Lysis Buffer, and a commercially available Abcam Lysis Buffer were tested using multiple blood donor samples (n=5). Additionally, in a two-step repeated pellet lysis process, a WBC pellet was first lysed with Abcam Lysis Buffer and then with 10% Triton® X-100 Lysis Buffer during the second lysis step. Table 1A shows the formulations of the lysis buffers used in the screening. Following pellet lysis, cell lysates were obtained, and the NE and PR3 activity, expressed as concentrations of active NE and PR3, respectively, in the cell lysates was quantified using the ProAxsis ELISA-based assays and the kinetic assays.

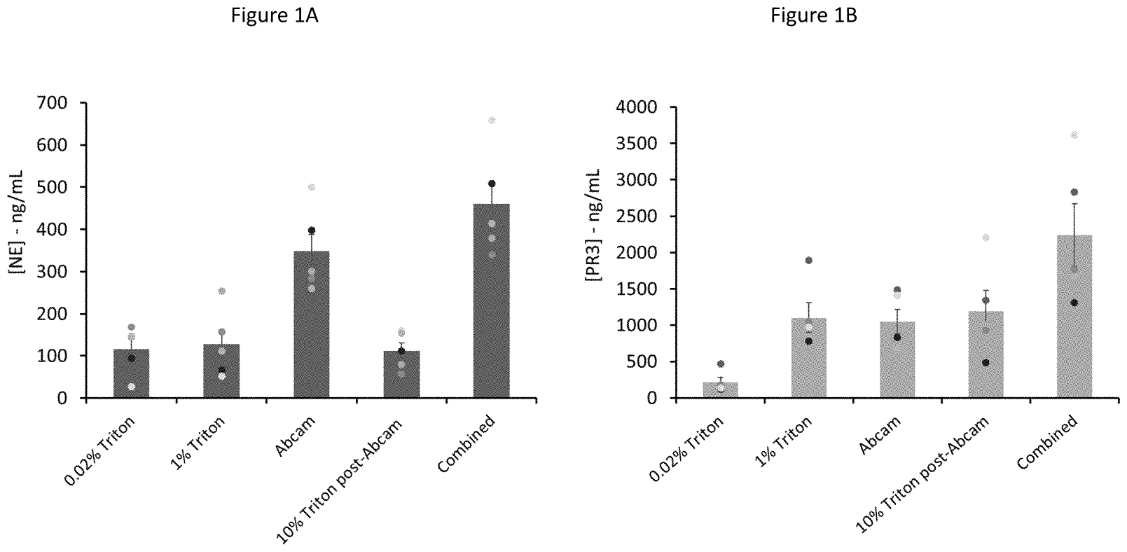

Compared to the ELISA-based assays, the kinetic assays exhibited greater sensitivity for NE and PR3 activity and less interference by the detergent and other agents carried over from the lysis buffers. As a result, the kinetic assays were performed and their results shown throughout the examples. As shown in FIG. 1A, 0.02% Triton® X-100 Lysis Buffer and 1% Triton® X-100 Lysis Buffer recovered similar amounts of active NE, whereas Abcam Lysis Buffer recovered more than three times as much active NE. As shown in FIG. 1B, 1% Triton® X-100 Lysis Buffer and Abcam Lysis Buffer achieved an equivalent recovery of active PR3, and five times more active PR3 recovery than 0.02% Triton® X-100 Lysis Buffer. The additional (second) lysis step with 10% Triton® X-100 Lysis Buffer following Abcam Lysis Buffer resulted in an additional recovery of active NE or active PR3 (FIGS. 1A and 1B).

### Example 2—Multi-Extractions of NE and PR3 from Human WBC Samples with Combinations of Lysis Buffers

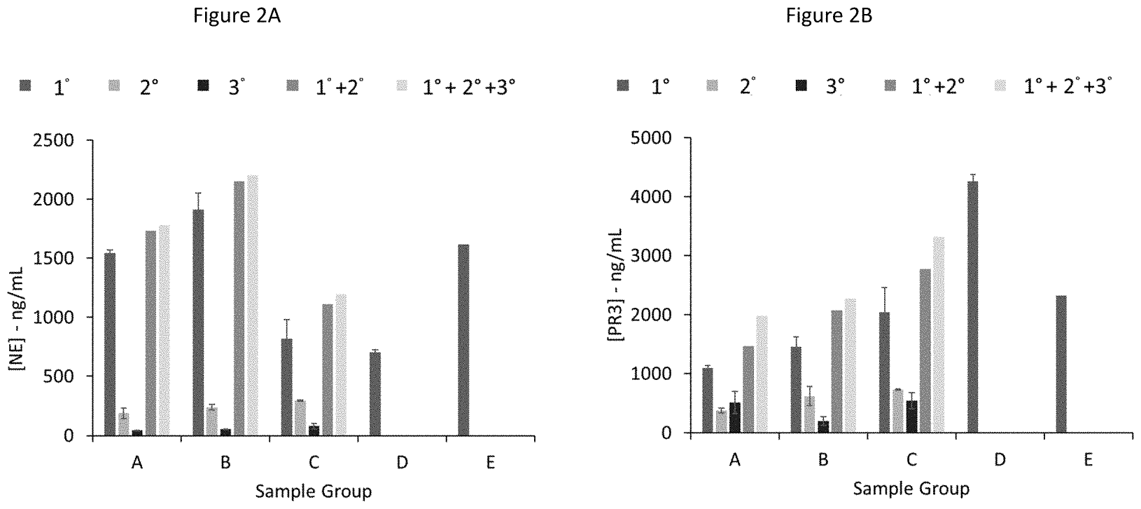

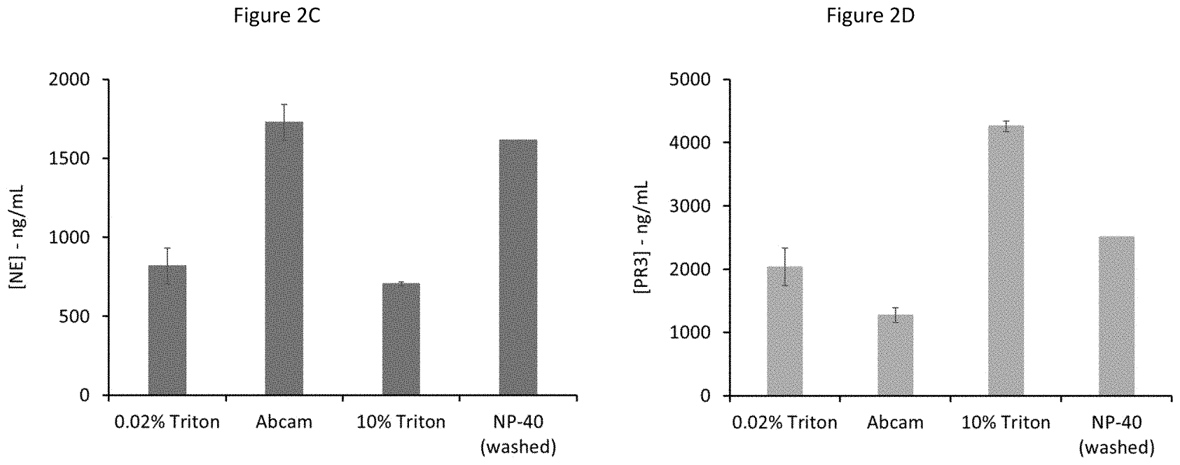

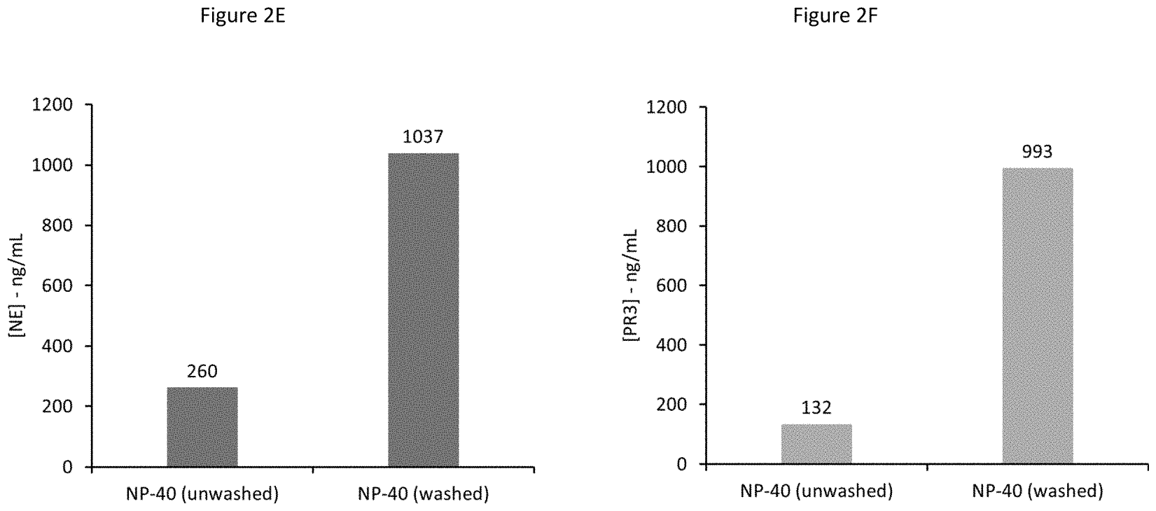

In the lysis buffer screening study, some buffers worked well in extracting NE, but worked poorly in extracting a different NSP or interfered with quantifying its activity. For example, Abcam Lysis Buffer showed the best recovery of active NE but the worst recovery for active PR3 (FIGS. 2C and 2D). Conversely, 10% Triton® X-100 Lysis Buffer recovered the most amount of active PR3 but the least amount of active NE (FIGS. 2C and 2D). To increase the recovery of NSPs while not sacrificing the quality of assay data due to interference, a multi-extraction process was explored that used different combinations of lysis buffers for extraction. Three different combinations of lysis buffers were tested in sample groups A, B, and C, respectively. Of each combination, the order of the lysis buffers used in a three-step repeated WBC pellet lysis process is shown in Table 2.

TABLE 2
Sample groups of human WBC pellets subjected to multi-extractions
of NE and PR3 with lysis buffer combinations

| Sample | Lysis buffer for pellet | Lysis buffer for pellet | Lysis buffer for pellet |
|---|---|---|---|
| Group | lysis step 1 | lysis step 2 | lysis step 3 |
| A | Abcam Lysis Buffer | 10% Triton ® X-100 | 0.02% Triton ® X-100 |
| | | Lysis Buffer | Lysis Buffer |
| B | Abcam Lysis Buffer | 0.02% Triton ® X-100 | 10% Triton ® X-100 |
| | | Lysis Buffer | Lysis Buffer |
| C | 0.02% Triton ® X-100 | Abcam Lysis Buffer | 10% Triton ® X-100 |
| | Lysis Buffer | | Lysis Buffer |

Additionally, a single (step) extraction with 10% Triton® X-100 Lysis Buffer was used as a control (sample group D). Lastly, NP-40 Lysis Buffer, which had not been previously evaluated, was tested under a single (step) extraction condition (sample group E). Two pellets from different donors were lysed in each sample group. Except for sample group E where NP-40 Lysis Buffer was used, the WBC pellets were unwashed. For sample group E using NP-40 Lysis Buffer, one of the two pellets was washed with PBS directly after RBC lysis during sample processing. Depending on the sample group, 1°, 2°, and 3° cell lysates or only 1° cell lysates were obtained following pellet lysis, and the NE and PR3 activity in the cell lysates was quantified using the NE and PR3 kinetic assays. Shown in FIGS. 2A and 2B, as well as Tables 3A and 3B, are data for the recovery of active NE and active PR3, respectively, from sample groups A-E based on the kinetic assay results. FIGS. 2A and 2B also show that the recovery of active NE and active PR3 from different donor WBC pellets exhibited normal variations.

TABLE 3A
Recovery of active NE following multi-extractions
or single extractions of sample groups A-E
Average [NE] - ng/mL (% of total)

| Sample | 1° cell | | 2° cell | 3° cell | 1° + 2° cell | |
|---|---|---|---|---|---|---|
| Group | lysate | | lysate | lysate | lysates | Total |
| A (Donor 12) | 1544 | (87%) | 188 (11%) | 44 (2%) | 1732 (98%) | 1776 (100%) |
| B (Donor 12) | 1913 | (87%) | 238 (11%) | 55 (2%) | 2151 (98%) | 2206 (100%) |
| C (Donor 12) | 819 | (69%) | 293 (25%) | 80 (7%) | 1113 (93%) | 1192 (100%) |
| D (Donor 12) | 706 | (100%) | — | — | — | — |
| E ((Donor 12, | 1614 | (100%) | — | — | — | — |
| pellet washed) | | | | | | |

TABLE 3B
Recovery of active PR3 following multi-extractions
or single extractions of sample groups A-E
Average [PR3] - ng/mL (% of total)

| Sample | 1° cell | 2° cell | 3° cell | 1° + 2° cell | |
|---|---|---|---|---|---|
| Group | lysate | lysate | lysate | lysates | Total |
| A (Donor 12) | 1092 (55%) | 375 (19%) | 510 (26%) | 1467 (74%) | 1978 (100%) |
| B (Donor 12) | 1455 (64%) | 618 (27%) | 200 (9%) | 2073 (91% | 2272 (100%) |
| C (Donor 12) | 2038 (62%) | 733 (22%) | 539 (16%) | 2772 (84%) | 3311 (100%) |
| D (Donor 12) | 4257 (100%) | — | — | — | — |
| E ((Donor 12, | 2321 (100%) | — | — | — | — |
| pellet washed) | | | | | |

Data from FIG. 2A and Table 3A show that multi-extractions of sample groups A and B with the buffer combinations, where Abcam Lysis Buffer was used in the first pellet lysis step, achieved comparable recovery of active NE, obtaining approximately 90-100% of total recoverable active NE after first two lysis steps (see active NE recovered in 1°+2° cell lysates).

Data from FIG. 2B and Table 3B show that multi-extractions of sample groups A-C with the buffer combinations recovered approximately 75-90% of total recoverable active PR3 after the first two lysis steps (see active PR3 recovered in 1°+2° cell lysates). As for total recovery of active PR3, multi-extraction of sample group C with its lysis buffer combination was approximately 1.5-fold better than multi-extraction of sample group A or B with their respective lysis buffer combinations. Among all of the extraction processes tested in sample groups A-E, single extraction of group D with 10% Triton® X-100 Lysis Buffer recovered the most amount of active PR3. By comparison, multi-extractions of groups A-C recovered approximately 40-80% of active PR3 recovered from the single extraction of group D.

When single extraction of group E using NP-40 Lysis Buffer was performed, a gel-like substance was formed, causing difficulty in pelleting the cell debris and the gel-like substance and in fully isolating and recovering the non-viscous supernatant. The difficulty was more severe with the unwashed WBC pellet. Accordingly, the washed WBC pellet lysed with NP-40 Lysis Buffer gave rise to four times more recovery of active NE and eight times more recovery of active PR3 than the unwashed pellet counterpart, as shown in FIGS. 2E and 2F. Without wishing to be bound by theory, pre-lysis wash of a WBC pellet may have removed interference or background from the RBC lysis, and/or reduced the formation of the gel-like substance, thus contributing to the increased recovery of NE and PR3. As shown in FIGS. 2C and 2D, single extraction (with a single, first lysis step) of a washed WBC pellet using NP-40 Lysis Buffer resulted in a comparable amount of active NE and more active PR3 recovered as compared to single extraction of unwashed WBC pellets with Abcam Lysis Buffer. As shown in FIG. 1A previously, single extraction with Abcam Lysis Buffer gave rise to better recovery of active NE as compared to single extraction with 0.02% Triton® X-100 Lysis Buffer or 1% Triton® X-100 Lysis Buffer.

### Example 3—Double Extractions of NE and PR3 from Human WBC Samples with NP-40 Lysis Buffer or NP-40 Lysis Buffer Followed by 10% Triton® X-100 Lysis Buffer

As shown in FIG. 1B previously, two-step WBC pellet lysis with Abcam Lysis Buffer followed by 10% Triton® X-100 Lysis Buffer gave rise to much improved recovery of active PR3 as compared to single-step lysis with Abcam Lysis Buffer. To evaluate NP-40 Lysis Buffer and 10% Triton® X-100 Lysis Buffer used alone or in combination in a two-step pellet lysis process, we determined recovery of active NE and active PR3 from WBC pellets subjected to double extractions with NP-40 Lysis Buffer at both lysis steps 1 and 2, with NP-40 Lysis Buffer at lysis step 1 followed by 10% Triton® X-100 Lysis Buffer at lysis step 2, or with 10% Triton® X-100 Lysis Buffer at both lysis steps 1 and 2. 1° and 2° cell lysates were obtained following lysis step 1 and 2, respectively, and the NE and PR3 activity, expressed as concentrations of active NE and active PR3, in the cell lysates was quantified using the NE and PR3 kinetic assays. The WBC pellets were washed with Assay Buffer prior to double extractions, since the previous experiments showed that the recovery of NSPs was higher when a washed pellet was lysed with NP-40 Lysis Buffer (FIGS. 2E and 2F). The wash fractions were saved for NE and PR3 kinetic assays also, since WBCs may have ruptured and released cytoplasmic material during the freeze-thaw process.

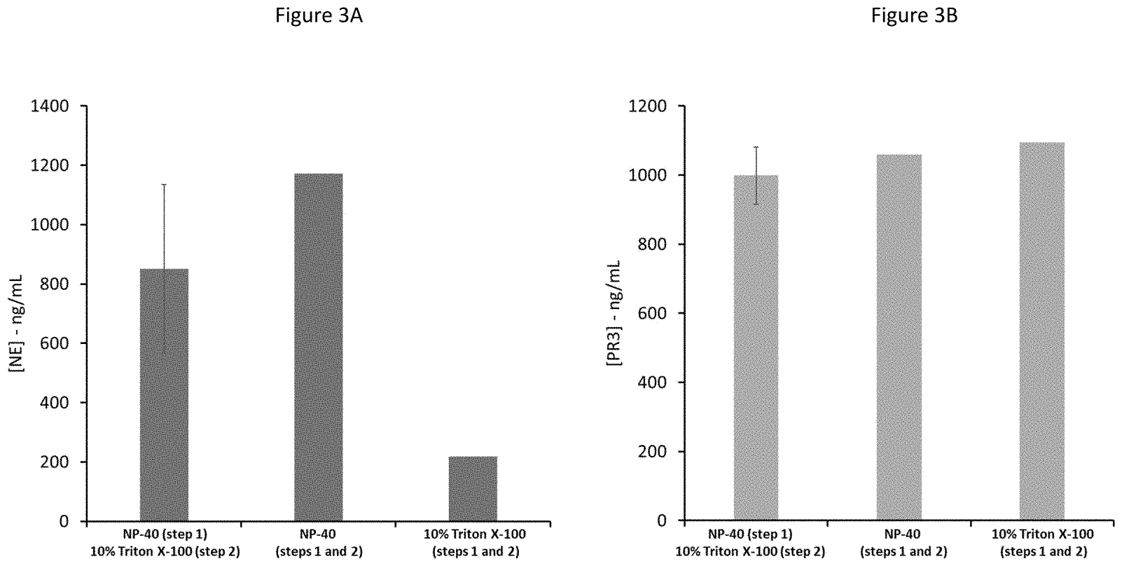

With regard to the recovery of active NE, the wash fraction showed approximately 10-25% of total recoverable NE activity (Table 4A). For samples lysed with NP-40 Lysis Buffer during pellet lysis step 1 and then with 10% Triton® X-100 Lysis Buffer during pellet lysis step 2, 2° cell lysate from lysis step 2 yielded less than 5% additional NE activity. For samples that underwent two-step pellet lysis with 10% Triton® X-100 Lysis Buffer, 2° cell lysate from lysis step 2 yielded less than 10% additional NE activity. For samples undergoing two-step pellet lysis with NP-40 Lysis Buffer, 2° cell lysate from lysis step 2 yielded approximately 30% additional NE activity (Table 4A). Overall, there was an approximately 1.4-fold and 5.4-fold greater recovery of active NE by double extractions with NP-40 Lysis Buffer than by double extractions with NP-40 Lysis Buffer followed by 10% Triton® X-100 Lysis Buffer, and by double extractions with 10% Triton® X-100 Lysis Buffer, respectively (FIG. 3A, Table 4A).

TABLE 4A
Recovery of active NE by double extractions with NP-40 Lysis
Buffer followed by 10% Triton ® X-100 Lysis Buffer, NP-40
Lysis Buffer only, or 10% Triton ® X-100 Lysis Buffer only
Average [NE] - ng/mL (% of total)

| Lysis buffer(s) used in two-step | Wash | 1° cell | 2° cell | |
|---|---|---|---|---|
| WBC pellet lysis | Fraction | lysate | lysate | Total |
| NP-40 Lysis Buffer (step 1) | 109 (13%) | 731 (86%) | 11 (1%) | 851 (100%) |
| 10% Triton ® X-100 Lysis Buffer | | | | |
| (step 2) | | | | |
| NP-40 Lysis Buffer | 100 (8%) | 760 (65%) | 311 (27%) | 1171 (100%) |
| (steps 1 and 2) | | | | |
| 10% Triton ® X-100 Lysis Buffer | 50 (23%) | 149 (68%) | 19 (9%) | 218 (100%) |
| (steps 1 and 2) | | | | |

TABLE 4B
Recovery of active PR3 by double extractions with NP-40 Lysis
Buffer followed by 10% Triton ® X-100 Lysis Buffer, NP-40
Lysis Buffer only, or 10% Triton ® X-100 Lysis Buffer only
Average [PR3] - ng/mL (% of total)

| Lysis buffer(s) used in two-step | Wash | 1° cell | 2° cell | |
|---|---|---|---|---|
| WBC pellet lysis | Fraction | lysate | lysate | Total |
| NP-40 Lysis Buffer (step 1) | 194 (19%) | 448 (45%) | 357 (36%) | 999 (100%) |
| 10% Triton ® X-100 Lysis Buffer | | | | |
| (step 2) | | | | |
| NP-40 Lysis Buffer | 248 (23%) | 600 (57%) | 212 (20%) | 1060 (100%) |
| (steps 1 and 2) | | | | |
| 10% Triton ® X-100 Lysis Buffer | 74 (7%) | 769 (70%) | 251 (23%) | 1094 (100%) |
| (steps 1 and 2) | | | | |

With regard to recovery of active PR3, the wash fraction showed approximately 10-25% of total PR3 activity (Table 4B). For samples lysed with NP-40 Lysis Buffer during pellet lysis step 1 and then with 10% Triton® X-100 Lysis Buffer during pellet lysis step 2, 2° cell lysate from lysis step 2 yielded approximately 35% additional PR3 activity. For samples subjected to double extractions with 10% Triton® X-100 Lysis Buffer, 2° cell lysate from lysis step 2 yielded approximately 25% additional PR3 activity. For samples doubly extracted with NP-40 Lysis Buffer, 2° cell lysate from lysis step 2 yielded 20% additional PR3 activity (Table 4B). All three double extraction designs exhibited comparable recovery of active PR3 (FIG. 3B, Table 4B). Taken together, our data show that pre-lysis wash of WBC pellets, collection of the resultant wash fractions for NE and PR3 activity assay, and double extractions of washed WBC pellets with NP-40 Lysis Buffer give rise to unexpected superior NE and PR3 recovery from human WBC samples.

### Example 4—Evaluation of the Effect of Enhanced Agitation Vs. An Additional Lysis Step with NP-40 Lysis Buffer on NE and PR3 Recovery from Human WBC Samples

Since physical disruption (e.g., agitation) of WBCs may also affect NSP recovery, we determined the effect of enhanced agitation via manual pipetting on NSP recovery from four different donor WBC pellet samples (B01-B04). In the previous examples, WBC pellets were physically agitated by pipetting the lysis buffer/pellet mixture ten times during each pellet lysis step. In this example, half the pellet from each donor sample was lysed with ten manual pipette agitations (referred to as “control half pellet”), and the other half was lysed with twenty manual pipette agitations (referred to as “half pellet with enhanced agitation”). The control half pellet was subjected to a three-step repeated pellet lysis process using NP-40 Lysis Buffer, with 1°, 2°, and 3° cell lysates collected following lysis step 1, 2, and 3, respectively. The half pellet with enhanced agitation was subjected to a two-step repeated pellet lysis process, also using NP-40 Lysis Buffer, with 1° and 2° cell lysates collected following lysis step 1, and 2, respectively. Prior to lysis step 1 with NP-40 Lysis Buffer, both the control half pellet and the half pellet with enhanced agitation were washed with Assay Buffer, with the wash fractions collected. The cell lysates and wash fractions were assayed for NE and PR3 activity to determine the recovery of active NE and PR3.

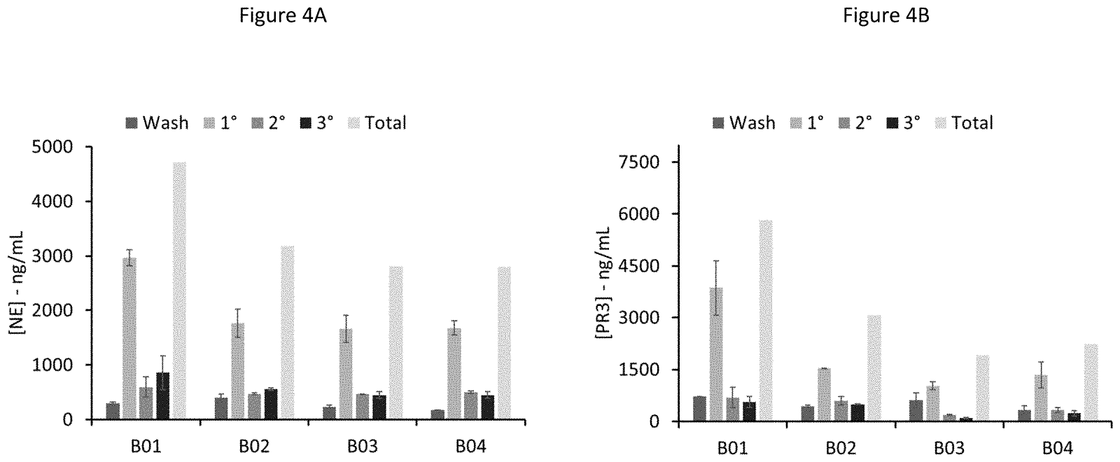

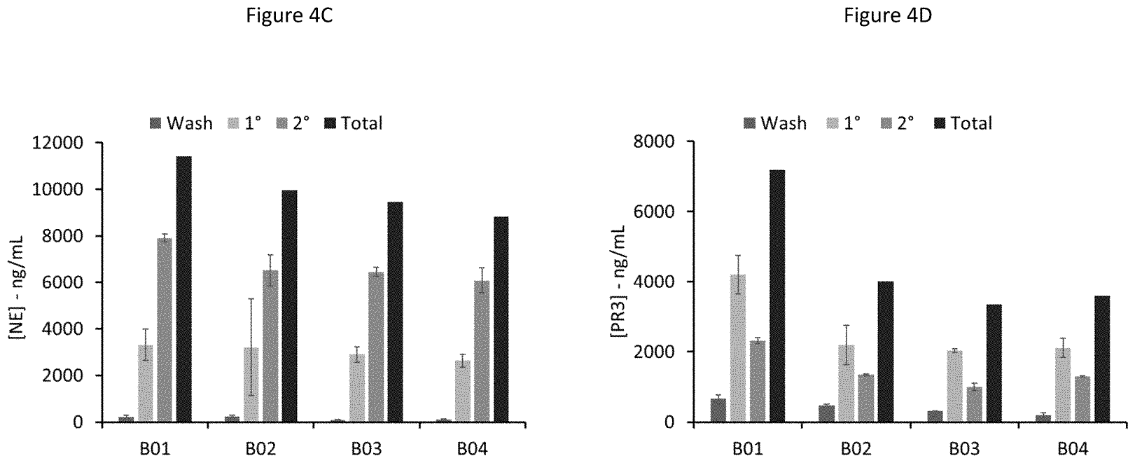

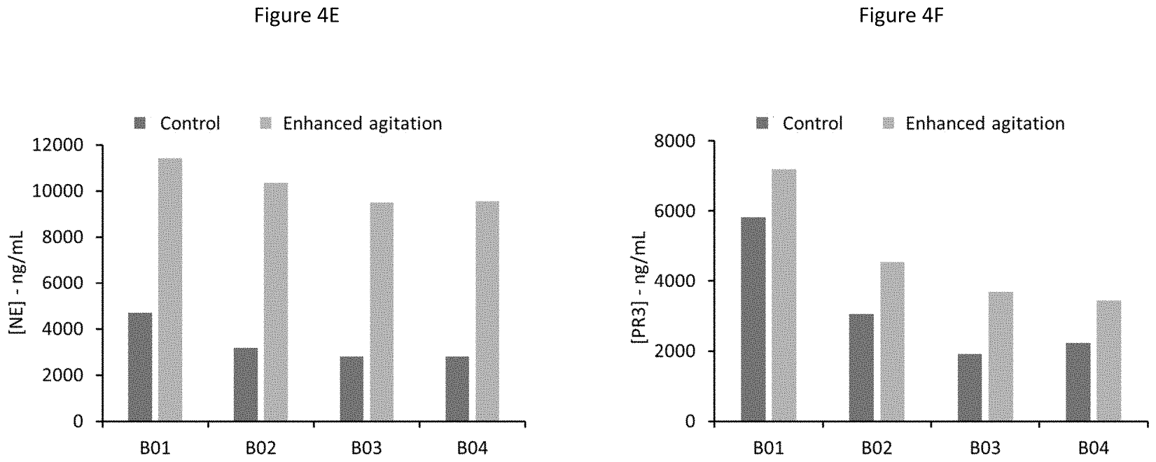

With the control half pellet, the wash fraction contained approximately 10% of the total recoverable active NE (Table 5A). 1°, 2°, and 3° cell lysates contained approximately 60%, 16%, and 17% of total active NE recovered, respectively (FIG. 4A, Table 5A). With the half pellet with enhanced agitation, the wash fraction contained less than 5% of the total active NE recovered. 1° and 2° cell lysates contained approximately 30% and 70% of the total active NE recovered, respectively (FIG. 4C, Table 5A). With respect to the amount of active NE recovered in the wash fraction and 1° cell lysate combined, enhanced agitation with twice the amount of pipetting during the first lysis step led to an average of 55% more recovery of active NE for 3 of the 4 donors (i.e., B02, B03, and B04), and approximately 8% more recovery of active NE for the remaining donor (B01, Table 5A). Two-step pellet lysis with enhanced agitation resulted in an approximately 3-fold greater total recovery of active NE than three-step pellet lysis with half the amount of agitation at each lysis step (FIG. 4E, Table 5A).

TABLE 5A
Effect of enhanced agitation vs. an additional lysis step using NP-40 Lysis Buffer on the
recovery of active NE from human WBC samples
Average [NE] - ng/mL (% of total)

| | Control half pellet subjected to three-step | | | | | Half pellet with enhanced agitation | | | |
|---|---|---|---|---|---|---|---|---|---|
| | repeated pellet lysis with 10 agitations per lysis | | | | | subjected to two-step repeated pellet | | | |
| | step | | | | | lysis with 20 agitations per lysis step | | | |
| Donor | Wash | 1º cell | 2º cell | 3º cell | | Wash | 1º cell | 2º cell | |
| ID | Fraction | lysate | lysate | lysate | Total | Fraction | lysate | lysate | Total |
| B01 | 294 (6%) | 2964 | 595 | 856 | 4707 | 207 (2%) | 3310 | 7909 | 11425 |
| | | (63%) | (13%) | (18%) | (100%) | | (29%) | (69%) | (100%) |
| B02 | 399 | 1762 | 471 | 552 | 3184 | 239 (2%) | 3201 | 6514 | 9954 |
| | (13%) | (55%) | (15%) | (17%) | (100%) | | (31%) | (67%) | (100%) |
| B03 | 235 (8%) | 1661 | 466 | 449 | 2812 | 88 (1%) | 2903 | 6459 | 9449 |
| | | (59%) | (17%) | (16%) | (100%) | | (31%) | (68%) | (100%) |
| B04 | 176 (6%) | 1675 | 499 | 449 | 2798 | 105 (1%) | 2638 | 6088 | 8831 |
| | | (60%) | (18%) | (16%) | (100%) | | (28%) | (71%) | (100%) |

Similar results were obtained for the recovery of active PR3. Specifically, with the control half pellet, the wash fraction contained approximately 20% of the total active PR3 recovered (Table 5B). 1°, 2°, and 3° cell lysates contained approximately 60%, 14%, and 10% of the total active PR3 recovered, respectively (FIG. 4B, Table 5B). With the half pellet with enhanced agitation, the wash fraction contained approximately 10% of the total active PR3 recovered, and 1° and 2° cell lysates contained approximately 55% and 35% of the total active PR3 recovered, respectively (FIG. 4D, Table 5B). With respect to the amount of active PR3 recovered in the wash fraction and 1° cell lysate combined, enhanced agitation with twice the amount of pipetting during the first lysis step led to approximately 40% more recovery of active PR3 on average for 3 of the 4 donors (i.e., B02, B03, and B04), and approximately 7% more recovery of active PR3 for the remaining donor (B01, Table 5B). Two-step pellet lysis with enhanced agitation resulted in 1.5-fold greater total active PR3 recovered than three-step pellet lysis with half the amount of agitation at each lysis step (FIG. 4F, Table 5B).

TABLE 5B
Effect of enhanced agitation vs. an additional lysis step using NP-40 Lysis Buffer on the
recovery of active PR3 from human WBC samples
Average [PR3] - ng/mL (% of total)

| | Control half pellet subjected to three-step | | | | | Half pellet with enhanced agitation | | | |
|---|---|---|---|---|---|---|---|---|---|
| | repeated pellet lysis with 10 agitations per lysis | | | | | subjected to two-step repeated pellet | | | |
| | step | | | | | lysis with 20 agitations per lysis step | | | |
| Donor | Wash | 1º cell | 2º cell | 3º cell | | Wash | 1º cell | 2º cell | |
| ID | Fraction | lysate | lysate | lysate | Total | Fraction | lysate | lysate | Total |
| B01 | 711 | 3860 | 690 | 554 | 5815 | 675 | 4200 | 2308 | 7183 |
| | (12%) | (66%) | (12%) | (10%) | (100%) | (9%) | (58%) | (32%) | (100%) |
| B02 | 434 | 1534 | 595 | 495 | 3058 | 481 | 2194 | 1343 | 4018 |
| | (14%) | (50%) | (19%) | (16%) | (100%) | (11%) | (48%) | (41%) | (100%) |
| B03 | 611 | 1024 | 181 | 101 | 1918 | 313 (8%) | 2035 | 1001 | 3349 |
| | (32%) | (53%) | (9%) | (5%) | (100%) | | (55%) | (36%) | (100%) |
| B04 | 325 | 1338 | 328 | 232 | 2224 | 200 (6%) | 2106 | 1296 | 3603 |
| | (15%) | (60%) | (15%) | (10%) | (100%) | | (61%) | (33%) | (100%) |

### Example 5—Evaluation of NE and PR3 Recovery from Human WBC Samples Via Five-Step Repeated WBC Pellet Lysis with NP-40 Lysis Buffer Under Enhanced Agitation

In this example, pre-lysis washed WBC pellets were subjected to a five-step repeated pellet lysis process using NP-40 Lysis Buffer under enhanced agitation (i.e., twenty pipette agitations during each lysis step). Wash fractions, and 1°, 2°, 3°, 4° and 5° cell lysates were collected and assayed for NE and PR3 activity to determine the recovery of active NE and active PR3. Additionally, in accordance with a reference extraction method currently practiced by the contract research industry, unwashed WBC pellets from the same donors were subjected to single (step) lysis using 0.02% Triton® X-100 Lysis Buffer under half the amount of agitation (i.e., ten pipette agitations during the single lysis step). 1° cell lysates were collected and likewise assayed for NE and PR3 activity to determine the recovery of active NE and active PR3.

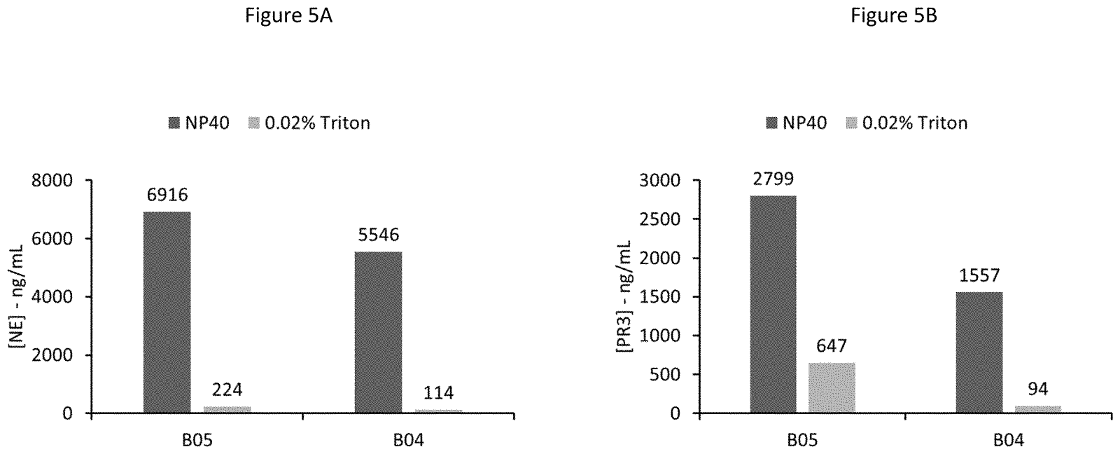

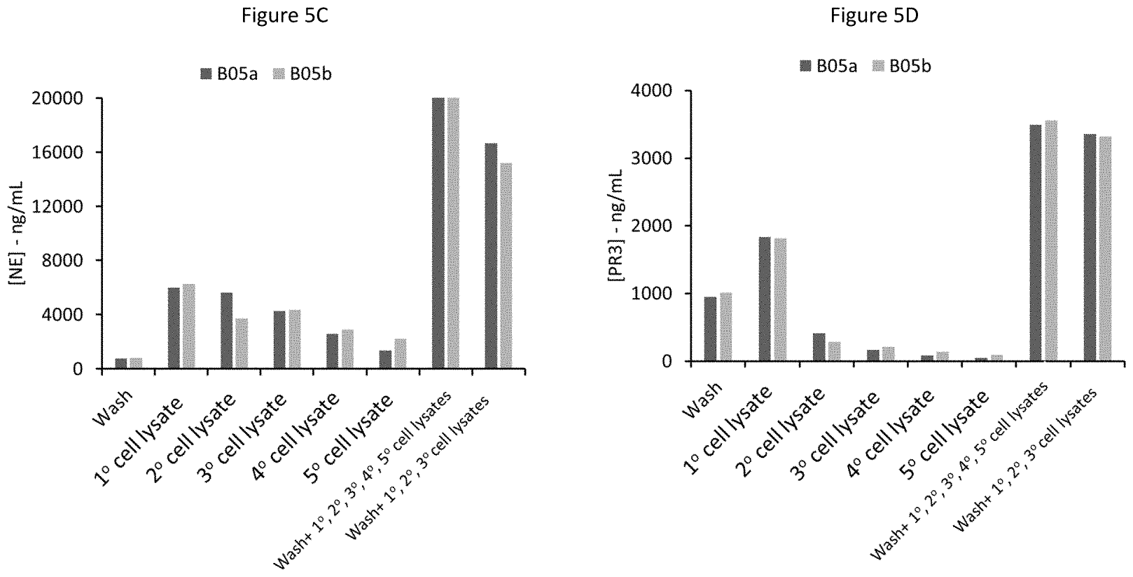

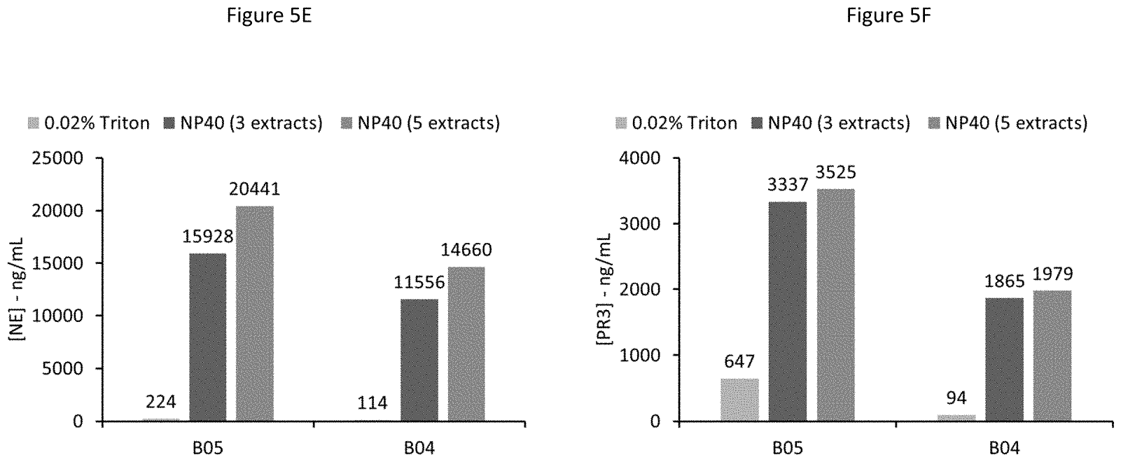

As shown in FIGS. 5A and 5B, 1° cell lysates obtained following the first lysis step of the repeated pellet lysis process with NP-40 Lysis Buffer under enhanced agitation contained as much as over 40 times the amount of active NE and over 15 times the amount of active PR3, as compared to 1° cell lysates obtained from single pellet lysis with 0.02% Triton® X-100 Lysis Buffer and reduced agitation, in accordance with the reference extraction method. Further, during the repeated pellet lysis process with NP-40 Lysis Buffer, additional active NE was recovered following each successive lysis step, with the wash fraction and 1°, 2°, and 3° cell lysates from the first three lysis steps recovering 70-80% of the total recoverable active NE (FIG. 5C). The total recoverable active NE was the sum of individual active NE amounts present in the wash fraction and 1°, 2°, 3°, 4°, and 5° cell lysates.

As for PR3 recovery with the five-step repeated pellet lysis process using pre-lysis washed WBC pellets and NP-40 Lysis Buffer under enhanced agitation, the wash fraction recovered approximately 30% of the total recoverable active PR3 (FIG. 5D). Additionally, the wash fraction combined with the 1°, 2°, and 3° cell lysates obtained from first three lysis steps recovered greater than 90% of the total recoverable active PR3 (FIG. 5D). The total recoverable active PR3 was the sum of individual active PR3 amounts present in the wash fraction and 1°, 2°, 3°, 4°, and 5° cell lysates.

We observed an overall increase in the recovery of active NE and active PR3 by as much as 100-fold and 20-fold, respectively, when washed WBC pellets were subjected to three or five repeated lysis steps with NP-40 Lysis Buffer under enhanced agitation, as compared to the recovery of active NE and active PR3 with the reference extraction method, where unwashed WBC pellets were lysed only once with 0.02% Triton® X-100 Lysis Buffer under 50% less agitation (FIGS. 5E and 5F). Additionally, the pre-lysis wash and multi-step repeated pellet lysis process yielded highly consistent results for the recovery of active NE and active PR3 when implemented on duplicate donor pellet samples (B05a and B05b), indicating that the NE and PR3 extraction method is robust and reproducible (FIGS. 5C and 5D). Taken together, our data show that pre-lysis wash of a WBC pellet and a multi-step repeated pellet lysis process with NP-40 Lysis Buffer and enhanced agitation contribute to superior recovery of NE and PR3 from human WBC samples.

### Example 6—Evaluation of the Effect of Antifoam on Kinetic NE and PR3 Assays

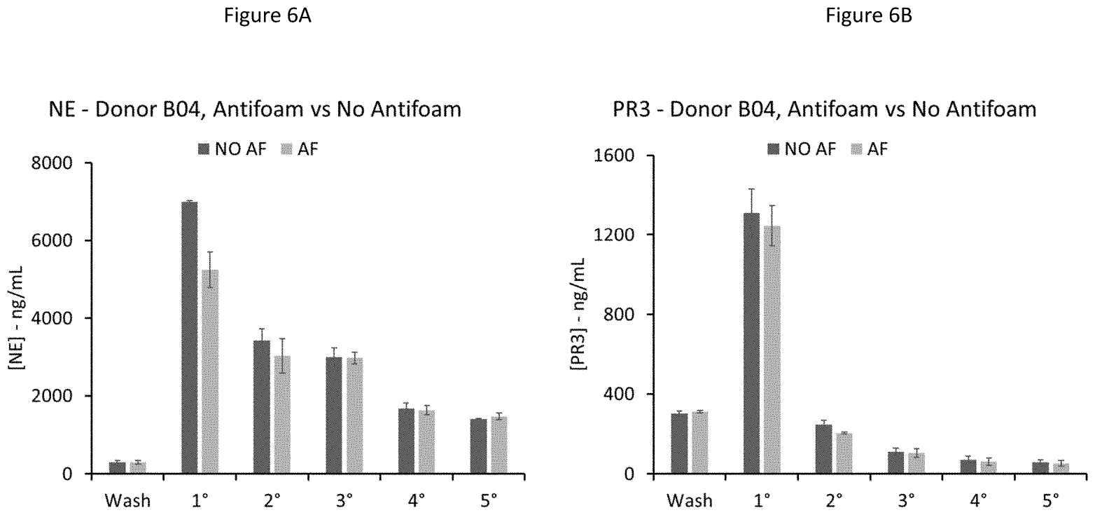

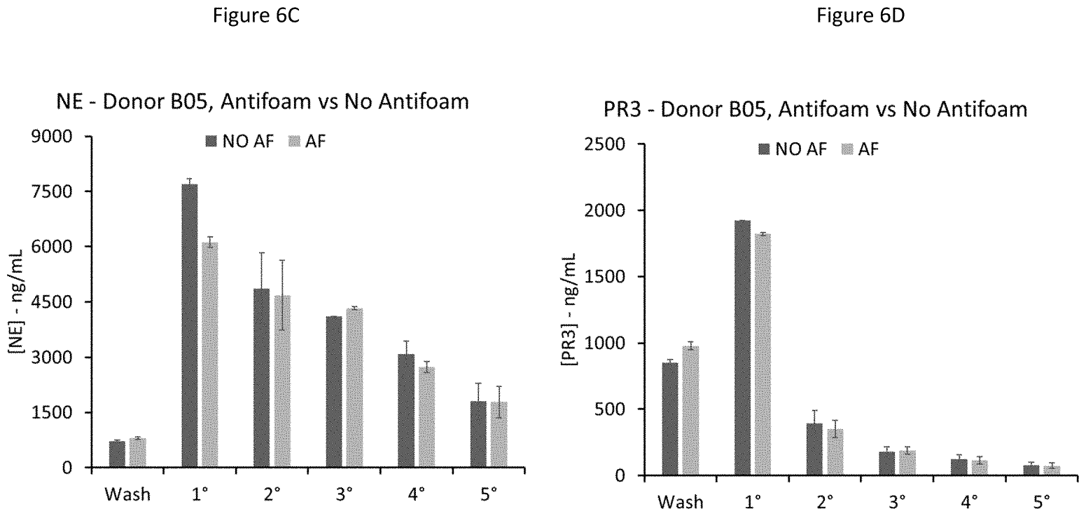

Since detergent (e.g., NP-40) was used to lyse WBC pellets, the detergent was present in cell lysates and hence carried over to the kinetic NE and PR3 assays, where the detergent may form bubbles that could alter the fluorescence readings by a plate reader. In order to mitigate that risk, we determined if use of an antifoam could decrease bubble formation in the wells of a plate and if the antifoam would interfere with the assays. To that end, WBC pellets from two different donors (B04 and B05) were washed, the wash fractions were collected, and cell lysates were made with the washed pellets according to the five-step repeated pellet lysis process using NP-40 Lysis Buffer under enhanced agitation, as described in Example 5. When the standards and the samples were prepared with the wash fractions and cell lysates for the kinetic NE and PR3 assays, an antifoam was added to the DMSO diluent for half the samples and standards. The samples and standards with the antifoam were compared to their counterparts without the antifoam. Except for the kinetic NE assay with 1° cell lysates (obtained from the first lysis step), the presence of the antifoam exhibited no interference with the NE and PR3 kinetic assays (FIGS. 6A-6D). Thus, addition of antifoam to prevent bubble formation improves the reliability of the NE and PR3 kinetic assays.

### Example 7—Evaluation of Cell Lysate Pooling for Determination of NE and PR3 Activity

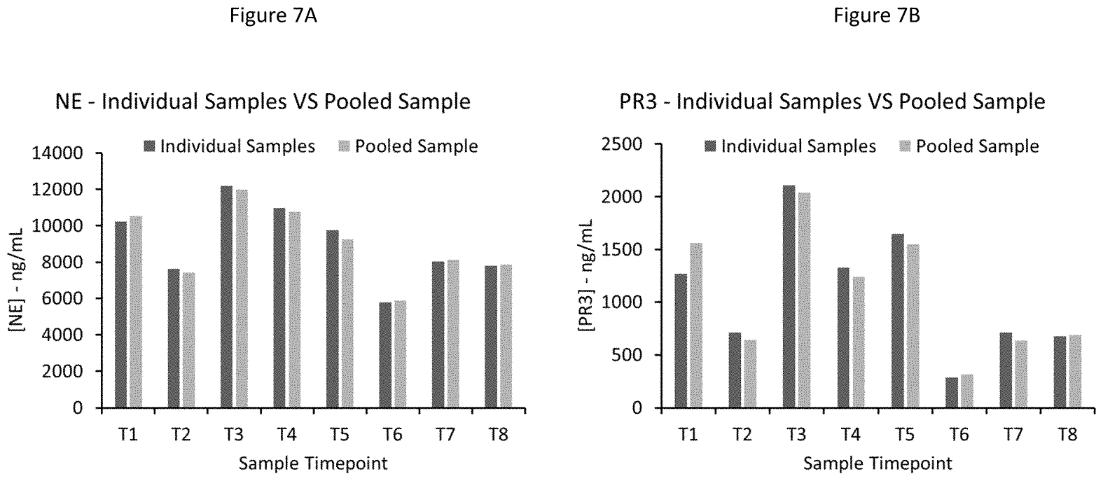

After NE and PR3 are extracted from a WBC pellet using a multi-step repeated pellet lysis process, total NE and PR3 activity in cell lysates may be determined by individually assaying each cell lysate from each lysis step for the enzyme activity and calculating the total activity. Alternatively, the total activity may be determined by pooling the cell lysates for a single NE or PR3 activity assay. Since assaying individual cell lysates would increase the number of assays needed and thus require more materials, reagents, and time, we determined whether the cell lysate pooling approach would produce a comparable result. WBC pellets were washed and then subjected to a three-step repeated pellet lysis process using NP-40 Lysis Buffer with enhanced agitation. Cell lysate from each lysis step was collected and assayed individually for NE and PR3 activity. Additionally, equal volumes of individual cell lysates were combined to yield a pooled cell lysate for a single NE or PR3 activity assay. As shown in FIGS. 7A and 7B, similar data for the recovery of active NE and active PR3, as well as a similar recovery trend across all of the sample timepoints, were obtained either by assaying the NE or PR3 activity of individual cell lysates and summing the activity, or by pooling individual cell lysates and assaying the NE or PR3 activity of the pooled cell lysate. In FIGS. 7A and 7B, each sample timepoint (from T1 to T8) corresponded to a different day in the course of a clinical trial when a whole blood sample was collected from a human subject.

### Example 8—Kinetic CatG Assay Development

This example describes the development of a kinetic CatG assay using various CatG substrates shown in Table 1B and both mouse bone marrow lysate samples and human WBC lysate samples comprising active CatG. This example also compares the kinetic CatG assay being developed with the commercially available AnaSpec's SensoLyte® Rh110 Cathepsin G Assay and ProteaseTag® Active CatG Immunoassay from ProAxsis with respect to specificity, sensitivity and accuracy. Specificity was determined by testing the substrate's ability to be cleaved by other NSP enzymes, including pure NE protein (Sigma, Cat. No E8140-1UN) and pure PR3 protein (Sigma, Cat. No SRP6309-25UG). The ability to be cleaved by enzymes other than CatG suggests low specificity. Sensitivity was assessed via differentiation of standard slopes and expressed as the slope of one standard as a percentage of the slope of the next higher concentration standard. Therefore, larger values indicate small changes in slope between standard concentrations indicating low sensitivity, whereas smaller values indicate more differentiation between slope values and potentially higher sensitivity. Additionally, the standard was created via 2-fold serial dilutions starting at 1 μg/mL; therefore, a linear curve would be expected to show 50% differentiation of standard slopes. Lastly, assay accuracy was assessed by spiking samples with pure CatG. 1. Testing of CatG substrates among Sigma kinetic CatG substrate (colorometric), Discovery Peptides kinetic CatG substrate (fluorometric), and Millipore Sigma kinetic CatG substrate (fluorometric) for the development of kinetic CatG assay

### Sigma Kinetic CatG Substrate (Colorometric)

The standard curve was observed to be relatively linear with consistent differentiation between standard slopes (49.9±3%, Table 6). This suggests that the assay is sensitive at distinguishing different sample concentrations over the range of 0.015625 to 1 μg/mL. Sigma's CatG substrate also showed minimal to no cleavage by NE and PR3 as the activity calculated was close to the negative control blank. Additionally, wash fractions did not show activity. Despite not being able to detect CatG activity in the wash fractions, there was measurable activity from human lysate samples. Samples tested with the CatG inhibitor showed less than 5% remaining CatG activity, further indicating minimal nonspecific cleavage of the substrate.

TABLE 6
Sensitivity and Standard Linearity for 2-fold Serial Dilution 0-1 μg/mL

| | | Discovery | Millipore |
|---|---|---|---|
| [Std], μg/mL | Sigma* | Peptides* | Sigma* |
| 0.5 | 52.7% | 29.3% | 60.6% |
| 0.25 | 50.0% | 23.4% | 64.6% |
| 0.125 | 51.4% | 35.6% | 95.5% |
| 0.0625 | 45.1% | 25.7% | 76.0% |
| 0.03125 | 48.2% | 58.9% | 72.3% |
| 0.015625 | 51.9% | 57.1% | 55.6% |
| Mean ± SD | 49.9 ± 2.8% | 38.4 ± 15.8% | 70.8 ± 14.3% |

$\begin{matrix}* Differentiation of standard slopes expressed as the slope of one \\ standard as a percentage of the slope of the next higher concentration \\ standard ( 1 , 0.5 , 0.25 , 0.125 , 0.0625 , 0.03125 , 0.015625 µg / mL ) , i . e . , \\ \frac{[ Std X ]}{[ Std X + 1 ]} \% .\end{matrix}$

Assay accuracy was assessed by spiking samples with 250 ng/mL CatG protein. In contrast to the unspiked wash fractions that exhibited no CatG activity, the spiked wash fractions showed measurable activity, at approximately 270 ng/mL, similar to the target concentration of the spiked CatG protein.

### Discovery Peptides Kinetic CatG Substrate (Fluorometric)

The standard curve showed less consistent differentiation between standards, and therefore was less linear (38.4±15.8%, Table 6). Discovery Peptides' CatG substrate also showed minimal to no cleavage by NE and PR3 as the activity calculated was close to the negative control blank. Additionally, the wash fractions and lysate samples that had been subjected to prolonged storage and multiple freeze-thaw cycles showed measurable activity. Samples tested with the CatG inhibitor showed less than 10% remaining CatG activity, indicating minimal nonspecific cleavage of the substrate.

Assay accuracy was assessed by spiking samples with 250 ng/mL CatG protein. The spiked samples showed measurable activity, at approximately 260 ng/mL, similar to the target concentration of the spiked CatG protein.

### Millipore Sigma Kinetic CatG Substrate (Fluorometric)

The standard curve showed varying slope trends, and it was noted that the substrate precipitated out of solution slightly when diluted, which may be a contributing factor. Additionally, the standard curve R-squared values were less than 0.985 instead of above 0.995 expected generally. The low R-squared value is most likely due to minimal differentiation between standard concentrations, further indicating the low assay sensitivity (70.8±14.3%, Table 6). While PR3 did not appear to cleave this substrate, the substrate was cleavable by NE. This suggests that the substrate is not specific to CatG, as only 80% inhibition was observed in the samples containing the CatG inhibitor. Therefore the 20% remaining activity may be due to NE cleavage of the substrate. Since the substrate did not demonstrate high specificity or high sensitivity, accuracy was not tested via the spiking of samples with CatG protein.

Based on the above findings, the Sigma substrate and Discovery Peptide substrate were the only substrates shown to be both specific and sensitive. In addition, both showed high accuracy in measuring spiked samples. Because the higher sensitivity (i.e., more consistent slope differentiation) and greater linearity of the standard curve were observed with the Sigma substrate compared to the Discovery Peptide substrate, the Sigma substrate was chosen for the CatG kinetic assay used in further studies.

### 2. Evaluation of SensoLyte® Rh110 Cathepsin G Assay Kit (Fluorometric)

Despite following the kit's instructions for standard curve preparation, overflow errors were observed. Additionally, the NE-containing sample showed overflow error due to too high fluorescence readings, indicating that the kit's CatG substrate can be cleaved by NE and is therefore not specific to CatG. Moreover, PR3 showed a minimal cleavage of substrate, further indicating that the kit's substrate has low specificity, and the activity detected by the assay kit in a sample may not be exclusively derived from CatG. In fact, the CatG inhibitor-containing samples did not show a reduction in activity, indicating that the activity detected is mainly that of NE and PR3 present in the samples. Since the kit did not demonstrate high specificity, accuracy was not tested via the spiking of samples with CatG protein.

3. Evaluation of the Kinetic CatG Assay Vs ELISA-Based ProteaseTag® Active CatG Immunoassay from ProAxsis

WBC pellets were processed for CatG extraction and activity determination using the kinetic CatG assay and the ProAxsis' ELISA-based assay, as summarized in Table 7.

TABLE 7
Summary of Cell Pellet Processing and Lysis

| | | Wash condition | | | | | | |
|---|---|---|---|---|---|---|---|---|
| | | Pre-lysis washed | | Washed immediately post-RBC lysis | | | | |
| | | Pellet Groups | | | | | | |
| | | A | B | C | D | E | F | G |
| RBC | RBC | + | + | + | + | + | + | + |
| Lysis | Lysis | | | | | | | |
| | Post RBC | — | — | +AB | +S | +S | +S | +S |
| | Lysis | | | | | | | |
| | Wash | | | | | | | |
| | Saline | — | — | — | — | — | +500 μL | — |
| | Added | | | | | | | |
| WBC | Wash | + | + | — | — | — | — | — |
| Pellet | with AB | | | | | | | |
| Lysis | Lysis | NP-40 | NP-40 | NP-40 | NP-40 | NP-40 | NP-40 | 0.02% |
| | Buffer | | | | | | | Triton X- |
| | | | | | | | | 100 |
| | # of Lysis | 3 | 3 | 3 | 3 | 3 (250 | 3 | 3 (250 μL |
| | Cycles | (pooled) | (pooled) | (pooled) | (pooled) | μL taken | (pooled) | taken from |
| | | | | | | from | | each cycle |
| | | | | | | each | | before |
| | | | | | | cycle | | pooling) |
| | | | | | | before | | |
| | | | | | | pooling) | | |

*AB = Assay Buffer;
S = 0.9% Saline

WBC pellets in groups A and B were subjected to a dual assay design that included a wash fraction activity assay and a lysate fraction activity assay, similar to that for determining NE and PR3 activity when NP-40 Lysis Buffer was used for extraction, as described in the previous examples.

To decrease the number of assays per NSP extraction, a single assay processing procedure was tested with pellets in groups C-G. This process involved washing the WBC pellet immediately post-RBC lysis to remove the excess RBC lysate residue that might cause interference. Pellets in group C were washed post-RBC lysis with Assay Buffer. Pellets in group D were washed post-RBC lysis with 0.9% saline. Pellets in group E were processed similar to the pellets in group D with additional evaluations of enzyme recovery after each lysis cycle. This was accomplished by saving a small portion of cell lysate from each lysis cycle for activity analysis before pooling the cell lysates. The effects of incomplete decanting of wash buffer, which could occur at a clinical site, were evaluated with pellets in group F, in which 500 μL saline was added back to each pellet after washing and decanting of the supernatant. For pellets in group G, 0.02% Triton X-100 Lysis Buffer instead of NP-40 Lysis Buffer was used for CatG extraction. 0.02% Triton X-100 Lysis Buffer has been widely used in the contract research industry to extract NSPs from various types of biological samples. A set of five WBC pellets from five different donors (Donors 1-5) was used in groups A and B combined and in each of groups C-G.

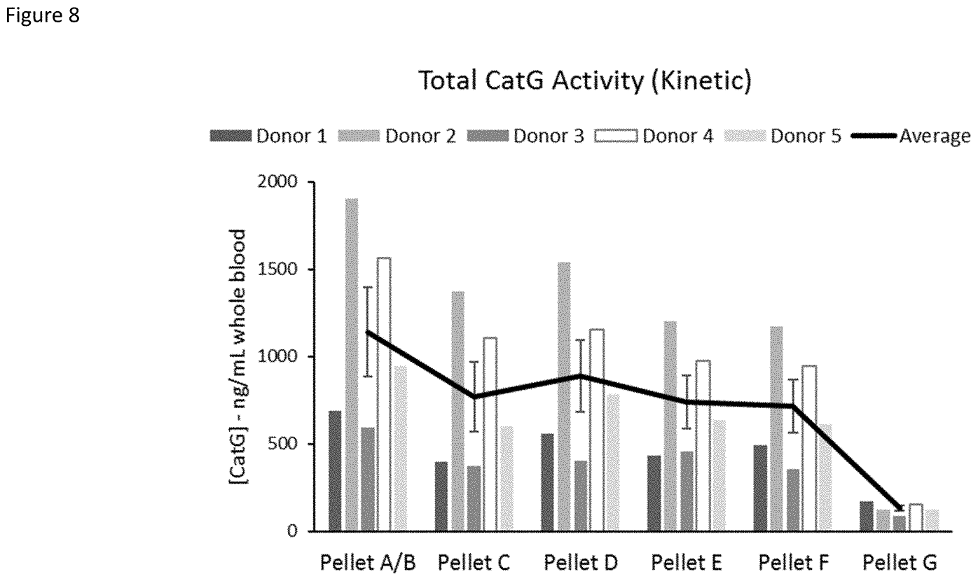

FIG. 8 shows the total CatG activity of WBC pellets in groups A-G determined by the kinetic CatG assay using Suc-AAPF-pNA peptide from Sigma as the substrate and expressed as active CatG concentrations. For pellets of groups A and B, the wash faction accounted for less than 15% of total CatG activity (data not shown). Different pellet processing procedures applied to the various pellet groups generated consistent inter-donor CatG activity results. Additionally, when comparing the dual assay procedure applied to pellets in groups A and B with the single assay procedure applied to pellets in groups C, D and E, washing immediately post RBC lysis resulted in approximately 25% loss of CatG activity, possibly due to a ‘dual assay’ artifact, i.e., a unidirectional system error caused by the conduct of two assays using different sample matrices (washing solution vs. cell lysate). This loss of activity was significant for group C pellets (P=0.0066) and group D pellets (P=0.0250); however, the activity of group E pellets was not found to be significantly lower than that of the group A/B pellets (P=0.0554).

Pellets in group F, whose processing procedure simulated an incomplete decanting of the wash buffer, a potential sample mishandling at a clinical site, exhibited a significantly diminished CatG activity by 20% as compared to its properly handled counterpart pellets of group D (P=0.0423). Lastly, group D pellets lysed with NP-40 Lysis Buffer showed a 3.5-fold better recovery of active CatG compared to group G pellets lysed with 0.02% Triton X-100 Lysis Buffer.

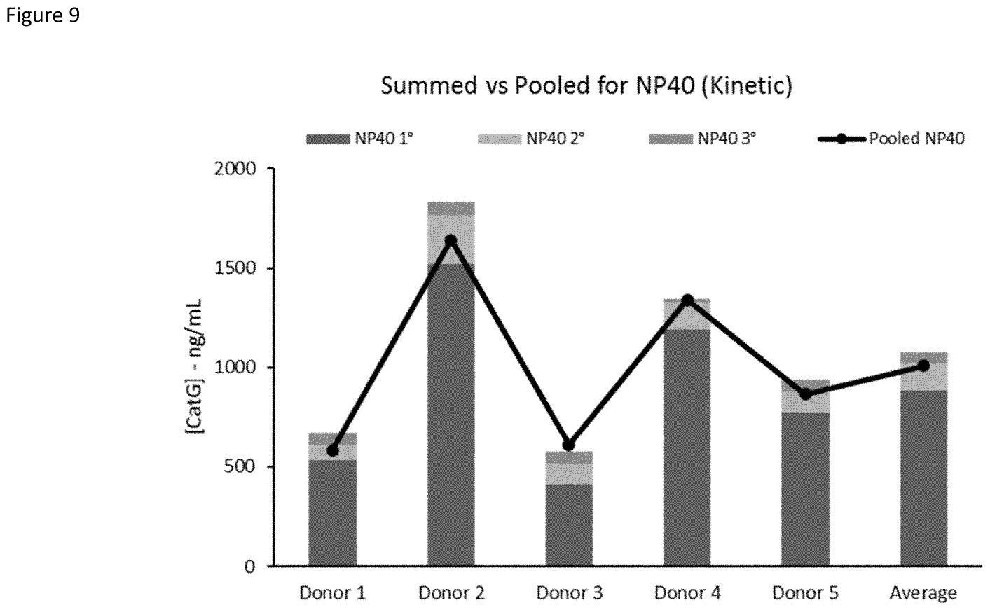

In addition to measuring the CatG activity with the pooled cell lysate fractions, CatG activity in individual cell lysate fractions from the primary, secondary and tertiary lysis of group E pellets was measured using the kinetic CatG assay. The purpose was two-fold: (1) to compare the sum of individual lysate fractions' CatG activity to the CatG activity of the pooled lysate fractions to determine the validity of the pooling approach; and (2) to determine if additional lysis steps were necessary to extract most of the active CatG. FIG. 9 shows the results, with the stacked bars representing the concentrations of active CatG in individual lysate fractions which were summed, and the line graph depicting the trend for the concentrations of active CatG in the pooled lysate fractions (corrected for pooling dilution) among WBC Donors 1-5, as well as the hypothetical “Average” donor with the corresponding average values of the five donors.

The summed and pooled active CatG concentrations are relatively close to each other and follow the same inter-donor trend as in FIG. 8, suggesting that pooling lysate fractions is a valid approach for the kinetic CatG assay (FIG. 9). Primary cell lysates contained approximately 81% of total active CatG. Secondary and tertiary cell lysates contained approximately 13% and 6% of total active CatG extracted, respectively, indicating that a near complete extraction was achieved after two lysis cycles.

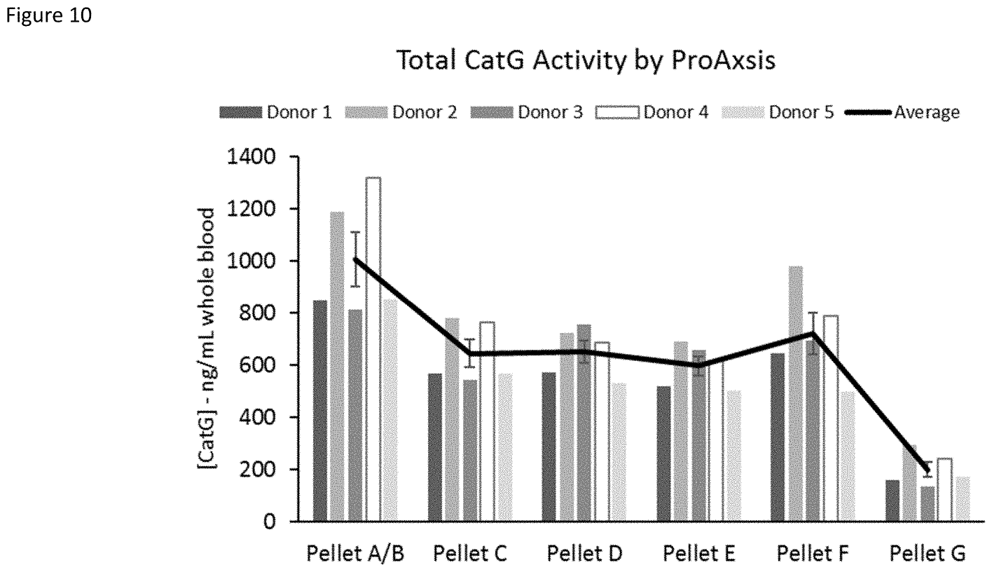

To compare the kinetic CatG Assay with the ELISA-based ProteaseTag® Active CatG Immunoassay from ProAxsis, duplicate WBC pellets of groups A-G were processed and lysed under the conditions prescribed in Table 7 and their CatG activity, also expressed as active CatG concentrations, determined by the ProAxsis' CatG assay. FIG. 10 shows the total CatG activity data. For pellets in groups A and B, the wash faction accounted for less than 20% of total CatG activity (data not shown). Additionally, when comparing the dual assay procedure applied to pellets of groups A and B with the single assay procedure applied to pellets of groups C, D and E, washing immediately post RBC lysis resulted in approximately 35% loss of CatG activity, possibly due to the ‘dual assay’ artifact discussed above. However, less consistency in inter-donor CatG activity results was observed among the various pellet groups subjected to different pellet processing procedures. Pellets of groups D and E showed an inter-donor trend different than pellets of the other groups processed and lysed under other conditions. This indicates that the ProAxsis' CatG assay is less reliable under different pellet processing procedures than the kinetic CatG assay.

Pellets of group F did not show a diminished CatG activity compared to pellets of group D, indicating that incomplete decanting of wash buffer would not affect the quantification of active CatG using the ProAxsis' CatG Assay.

Lastly, group D pellets lysed with NP-40 Lysis Buffer likewise showed a 3.5-fold better recovery of active CatG compared to group G pellets lysed with 0.02% Triton X-100 Lysis Buffer.

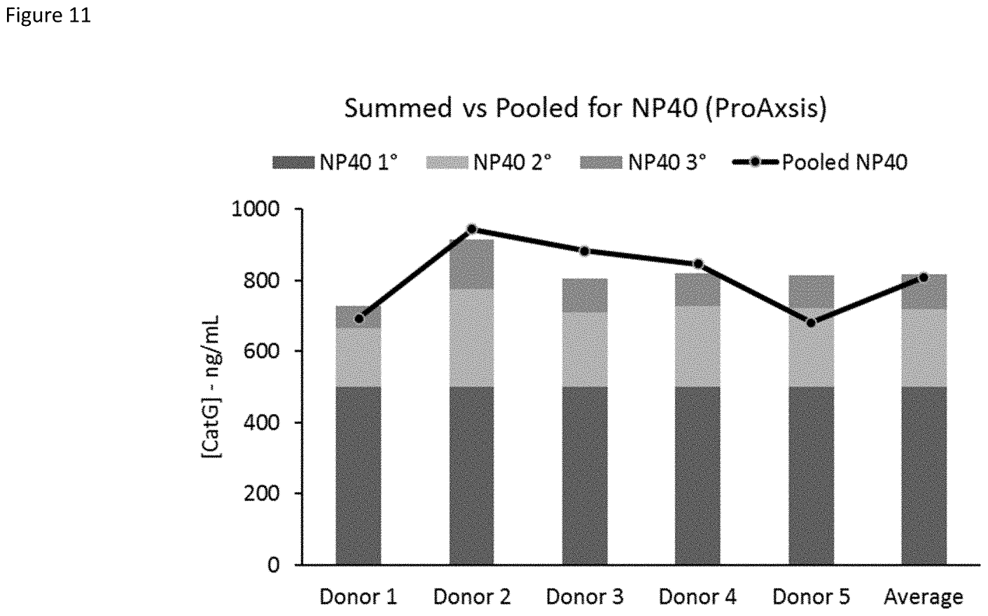

In addition to measuring the CatG activity with the pooled cell lysate fractions, CatG activity in individual cell lysate fractions from the primary, secondary and tertiary lysis of Pellet E was measured using the ProAxsis' CatG Assay. FIG. 11 shows the results, with the stacked bars representing the concentrations of active CatG in individual lysate fractions which were summed, and the line graph depicting the trend for the concentrations of active CatG in the pooled lysate fractions (corrected for pooling dilution) among WBC Donors 1-5, as well as the “Average” donor with the corresponding average values of the five donors.

The summed and pooled active CatG concentrations are relatively close to each other, suggesting that pooling lysate fractions is a valid approach for the ProAxsis' CatG Assay (FIG. 11). Primary cell lysate contained approximately 62% of total active CatG recovered. Secondary and tertiary cell lysates contained approximately 27 and 12% of total active CatG extracted, respectively. The primary cell lysates may actually have contained more than 62% of the total active CatG, as those samples resulted in overflow and therefore were approximated to be of the highest standard concentration.

In summary, the ProAxsis' CatG Assay and the kinetic CatG assay yielded similar results from the identical samples. However, the ProAxsis' CatG Assay appeared to have lower assay sensitivity, as different pellet processing procedures used led to less consistent inter-donor CatG activity results. Additionally, the ProAxsis' assay generated a sigmoidal standard curve that required multiple dilutions to ensure that samples fall within the standard range and thus required more time to prepare. In contrast, the kinetic CatG assay's standard curve was nearly linear, allowing for more flexibility of sample dilutions and standard curve range. Thus, the kinetic CatG assay provides improved sensitivity, consistency, and reliability over the ProAxsis' CatG Assay for the quantification of active CatG in WBC samples.

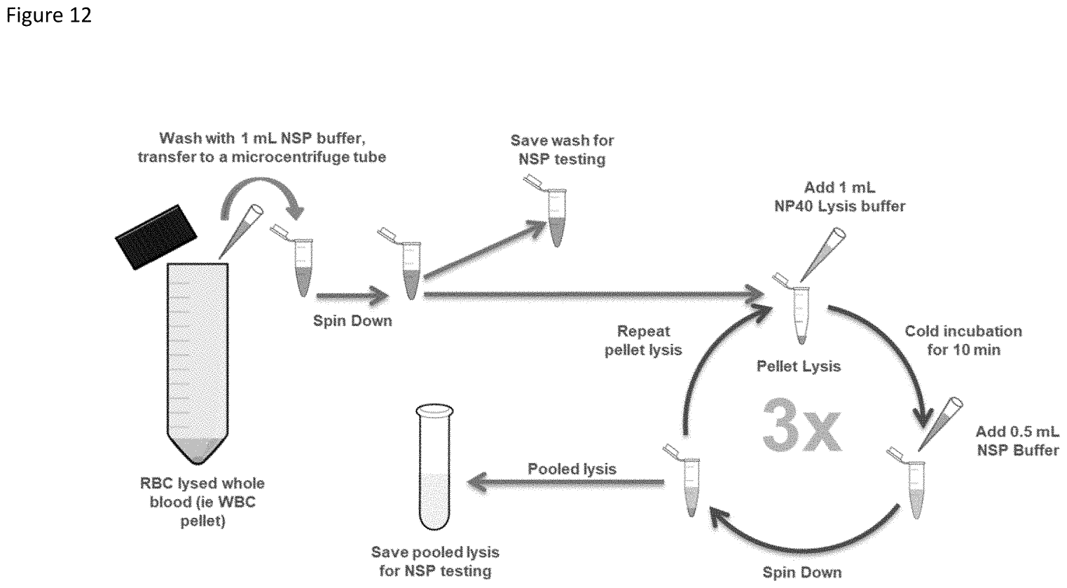

Taken together, the examples above demonstrate an efficient and reproducible method for extracting an NSP from WBC pellets, as illustrated in FIG. 12. Streamlined and compatible with downstream NSP activity assays, the method features pre-lysis or (immediate) post-RBC lysis wash of the pellets with NSP buffer (i.e., Assay Buffer) or 0.9% saline to decrease gelling and interference with the activity assays, collection and inclusion of the wash fraction for the NSP activity assays, multi-step (e.g., 3, 4, or 5-step) repeated lysis of the washed pellets using NP-40 Lysis Buffer to generate cell lysates containing extracted NSP, enhanced mechanical agitation during each lysis step, and addition of NSP buffer to each cell lysate to further reduce gelling and facilitate collection of cell lysate following spin-down, and pooling cell lysates for NSP activity assays.

### Example 9—Reduction of Active NSP Concentrations in WBC Samples Obtained from Patients with Non-Cystic Fibrosis Bronchiectasis Receiving Brensocatib Treatment was Associated with Improvements in Bronchiectasis Clinical Outcomes

We have conducted a phase 2, randomized, double-blind, placebo-controlled trial to assess the efficacy, safety and tolerability, and pharmacokinetics of (2S)—N-{(1S)-1-cyano-2-[4-(3-methyl-2-oxo-2,3-dihydro-1,3-benzoxazol-5-yl) phenyl] ethyl}-1,4-oxazepane-2-carboxamide (brensocatib) administered once daily for 24 weeks in patients with non-cystic fibrosis bronchiectasis (NCFBE). See the details and results of the trial in N Engl J Med. 383(22):2127-2137 (2020); incorporated herein by reference in its entirety for all purposes.

In the trial, subjects were randomized in a 1:1:1 ratio to 3 treatment arms to receive either (i) 10 mg brensocatib once daily; (ii) 25 mg brensocatib once daily, or (iii) matching placebo once daily. Following a screening visit (Visit 1) and a screening period of up to 6 weeks, subjects were randomized at Visit 2 (Day 1, “Baseline”) and returned thereafter for study visits at 2 weeks (Visit 3), 4 weeks (Visit 4), 8 weeks (Visit 5), 12 weeks (Visit 6), 16 weeks (Visit 7), 20 weeks (Visit 8), 24 weeks (Visit 9, end of treatment) and 28 weeks (Visit 10, end of study). During each visit, assessments and procedures were performed, including collection of blood and sputum samples at baseline and weeks 2, 4, 12, 24, and 28 for biomarker assessment. Study treatment occurred between Visits 2-9. The time to the first pulmonary exacerbation (primary end point), the rate of pulmonary exacerbations (secondary end point), change in concentration of active NE in sputum, and safety were assessed. Brensocatib treatment at both dosages prolonged the time to the first exacerbation as compared with placebo (p=0.03 for 10-mg brensocatib vs. placebo; p=0.04 for 25-mg brensocatib vs. placebo). In addition, brensocatib treatment resulted in a reduction in the frequency of pulmonary exacerbations as compared to placebo. Specifically, patients treated with brensocatib experienced a 36% reduction in the 10 mg arm (p=0.04) and a 25% reduction in the 25 mg arm (p=0.17). Change in concentration of active NE in sputum versus placebo from baseline to the end of the treatment period was also statistically significant (p=0.034 for 10 mg, p=0.021 for 25 mg), indicating an association between reduced NSP activity in the sputum and improvements in bronchiectasis clinical outcomes.

In this example, we further determined the changes from baseline in the concentrations of active PR3 and CatG in the same sputum samples obtained from the patients in the three treatment arms using the kinetic PR3 and CatG assays described in the previous examples, and compared the changes with those of active NE. Additionally, we extracted NE and PR3 from the white blood cells (WBCs) derived from the patients' blood samples, and determined the changes from baseline in the concentrations of active NE and PR3 in the WBC samples using the methods described in Example 7. We further studied the relationships between the changes in active NSP levels from the same sample type or from different sample types.

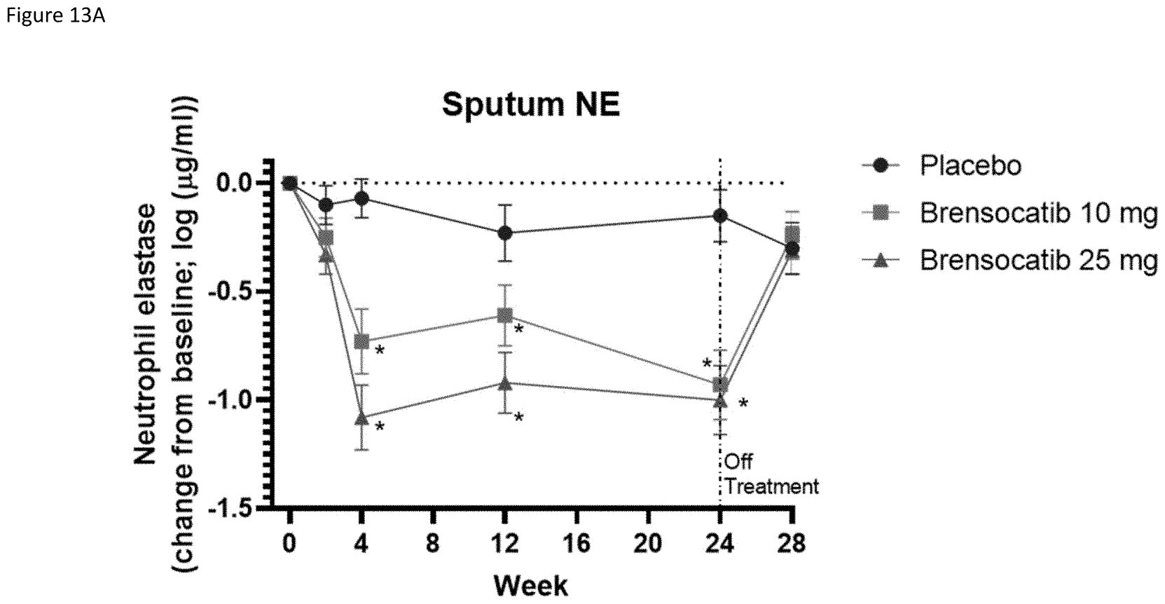

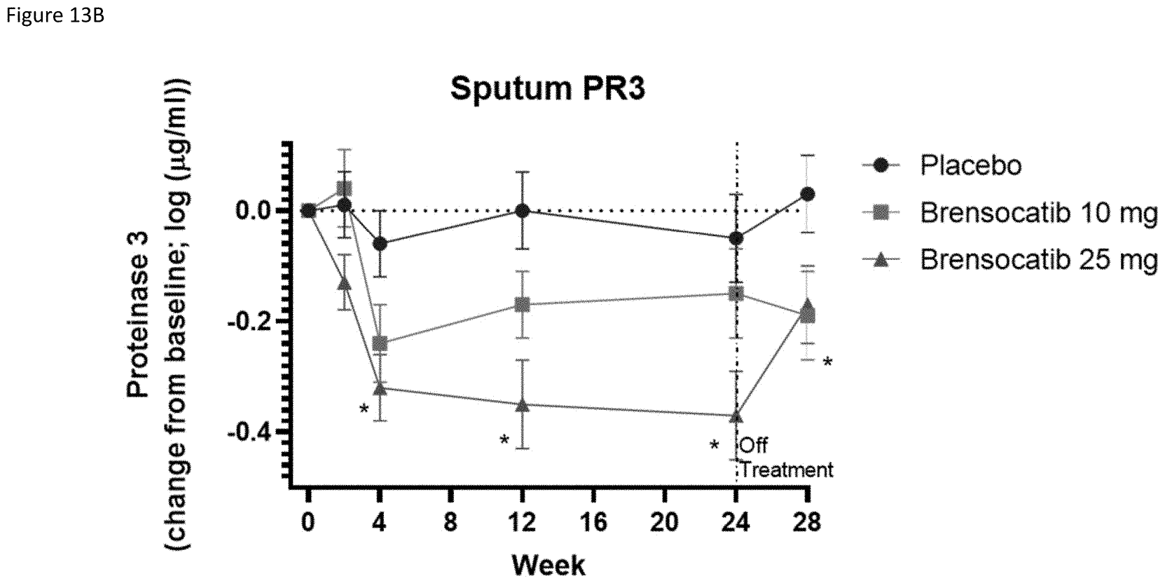

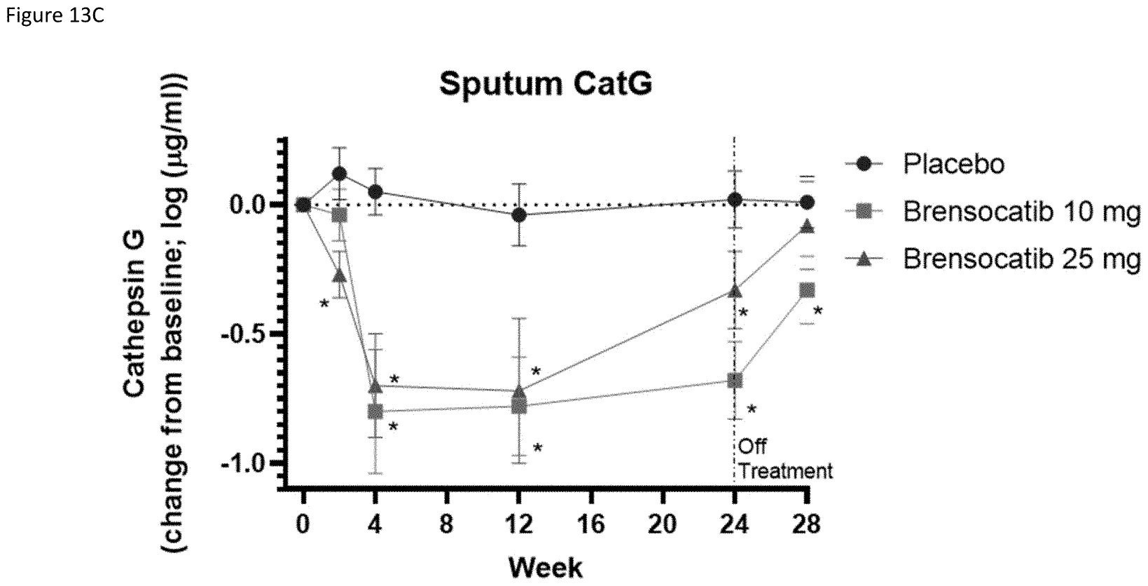

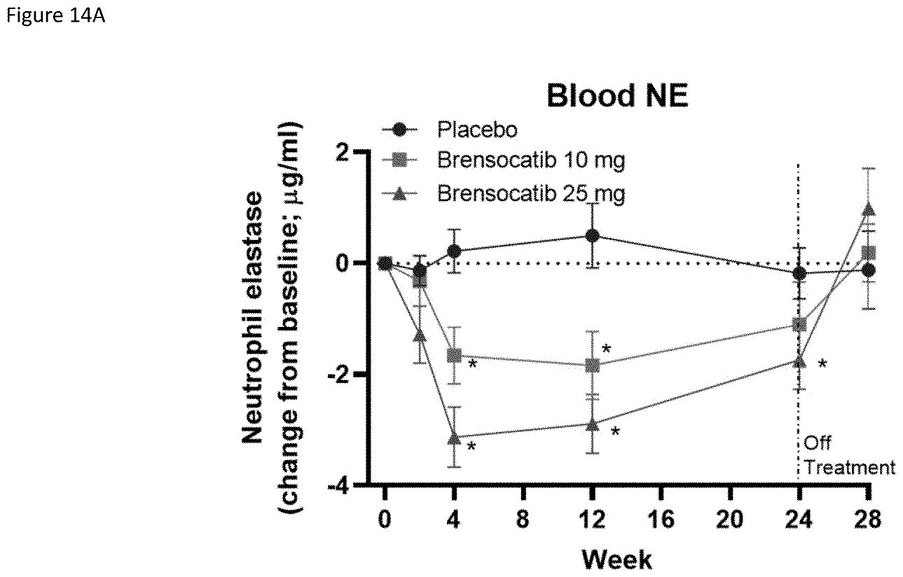

FIGS. 13A, 13B, and 13C show the changes from baseline (Week 0) in the sputum concentrations of active NE, PR3, and CatG, respectively. The baseline sputum concentrations of active NE, PR3, and CatG were each derived from the mean values observed during screening and day 1. The three active NSPs exhibited similar patterns of change in sputum concentrations. The sputum concentration of each active NSP was reduced by brensocatib treatment by week 4 in a dose-dependent manner, with a greater reduction in the 25 mg brensocatib arm than in the 10 mg brensocatib arm, and recovered through 4 weeks after the end of the treatment period. Among the three active NSPs, the concentration of active PR3 was the least reduced by the brensocatib treatment.

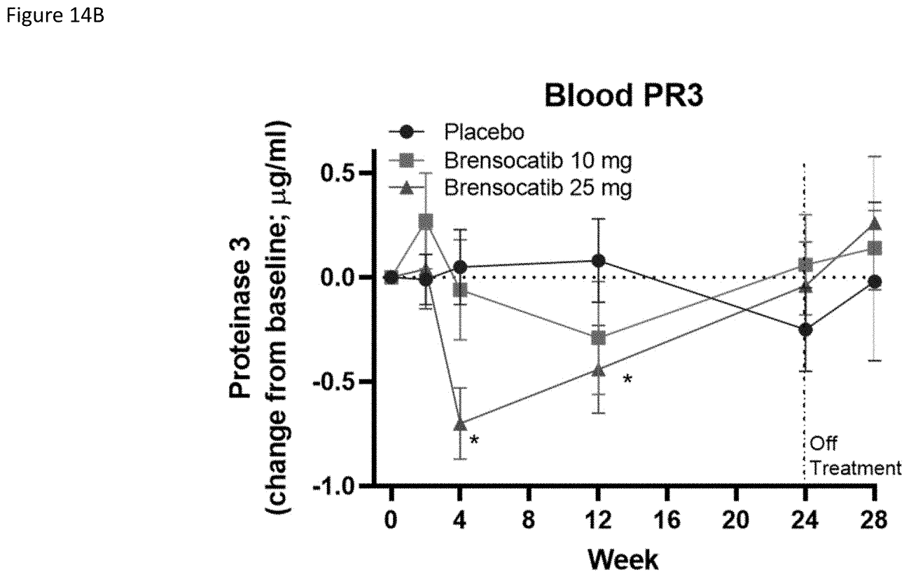

FIGS. 14A and 14B show the changes from baseline (Week 0) in the concentrations of active NE and PR3, respectively, in the patients' WBC samples. Active NE concentrations in the WBC samples were reduced by brensocatib treatment by week 4 in a dose-dependent manner, with the reductions persisting over the 24-week treatment period. The reduced active NE concentrations recovered to baseline levels about 4 weeks after the end of the treatment period (FIG. 14A). A similar trend was observed for the active PR3 concentrations, except that they were reduced by brensocatib treatment to a lesser extent than the active NE concentrations (FIG. 14B).

Taken together, brensocatib treatment reduced active NE, PR3, and CatG levels in the sputum samples originated from the lung, where active NE, PR3, and CatG are the primary drivers of chronic inflammation in NCFBE. Brensocatib treatment also reduced active NE and PR3 levels in WBCs with a similar time course and duration, although the reduction in the WBCs was less than the corresponding reduction in the sputum samples.

Table 8 shows percentage reductions from baseline of active NSP concentrations in WBC samples and in the sputum by week 4 of the brensocatib treatment period.

TABLE 8
Percentage reductions from baseline of active NSP concentrations
in WBC and sputum samples by week 4 of brensocatib treatment

| | Daily dose | % reduction from | % reduction from |
|---|---|---|---|
| Active | of brensocatib | baseline in | baseline in |
| NSP | treatment | WBC samples^{a} | sputum samples^{b} |
| NE | 10 mg | 19 | 86 |
| | 25 mg | 54 | 91 |
| PR3 | 10 mg | No reduction | 21 |
| | 25 mg | 34 | 53 |
| CatG | 10 mg | Not determined | 90 |
| | 25 mg | Not determined | 93 |

^{a}Reduction in arithmetic mean.
^{b}Reduction in geometric mean.

Week 4 was chosen as it was the first timepoint when we expected to see the full impact of brensocatib on the NSP activity. In both the WBC and sputum samples, brensocatib at the higher dose (25 mg) resulted in a greater reduction in active NSP levels. Additionally, the active NSP concentrations in the sputum were reduced to a greater extent than those in the WBCs. For example, in patients of the 10 mg brensocatib arm, active NE level was reduced 19% in the WBCs compared to 86% reduction in the sputum. In the 25 mg brensocatib arm, greater reductions at 5⁴% and 91% were observed in the WBCs and in the sputum, respectively.

Table 9 shows positive correlations between levels of active NSPs from the same sample type (i.e., from a WBC sample or from a sputum sample), as well as between levels of active NSPs from different sample types.

TABLE 9
Pearson r values of correlation between levels of active
NSPs from the same sample type and different sample types

| | Sputum | Sputum | Sputum | Blood | Blood |
|---|---|---|---|---|---|
| | NE | PR3 | CatG | NE | PR3 |
| Sputum NE | 1.00 | 0.64 | 0.87 | 0.31 | 0.18 |
| Sputum PR3 | 0.64 | 1.00 | 0.61 | 0.28 | 0.23 |
| Sputum | 0.87 | 0.61 | 1.00 | 0.36 | 0.22 |
| CatG | | | | | |
| Blood NE | 0.31 | 0.28 | 0.36 | 1.00 | 0.67 |
| Blood PR3 | 0.18 | 0.23 | 0.22 | 0.67 | 1.00 |

In Table 9, each of the five biomarkers (i.e., sputum NE, PR3, and CatG, and blood NE and PR3) are listed on both the top and the left side, with the perfect correlation of 1 along the diagonal line. Strong positive correlations were seen between two different NSPs from the sputum samples, ranging from 0.61 to 0.87. The strongest correlation was between the sputum levels of active CatG and active NE. We also observed a positive correlation between levels of blood NE and blood PR3. Additionally, we observed slightly lower positive correlations, between blood and sputum NSP levels, ranging from 0.18 to 0.36.

Because of the positive correlations among active NSP levels both within, and between, sputum and WBC samples, reductions of active NSP concentrations in WBC samples by brensocatib treatment in patients with bronchiectasis, like the corresponding reductions in the sputum, are associated with improvements in bronchiectasis clinical outcomes. Therefore, reduction in concentration of an active NSP (e.g., NE, PR3, CatG, and NSP4) in WBCs and the extent of the reduction can serve as a useful biomarker for determining effective brensocatib dosages and/or evaluating the efficacy of brensocatib treatment of NCFBE and other DPP1-mediated diseases as disclosed herein.

While the described invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the described invention. All such modifications are intended to be within the scope of the claims appended hereto.

Patents, patent applications, patent application publications, journal articles and protocols referenced herein are incorporated by reference in their entireties, for all purposes.

1. A method of extracting one or more neutrophil serine proteases (NSPs) from a sample comprising white blood cells (WBCs) obtained from a subject, the method comprising:
contacting the sample with a first aqueous medium comprising at least 0.01% (v/v) of a first nonionic surfactant to obtain a first cell lysate comprising a first NSP extract, and a first WBC residual, wherein the first NSP extract comprises the one or more NSPs,
separating the first cell lysate from the first WBC residual, to provide a first separated cell lysate comprising the first NSP extract,
contacting the first WBC residual with a second aqueous medium comprising at least 0.01% (v/v) of a second nonionic surfactant to obtain a second cell lysate comprising a second NSP extract, and a second WBC residual, wherein the second NSP extract comprises the one or more NSPs, and
separating the second cell lysate from the second WBC residual to provide a second separated cell lysate comprising the second NSP extract.

2. The method of claim 1, wherein contacting the sample with the first aqueous medium and contacting the first WBC residual with the second aqueous medium are each performed at a temperature of from about 0° C. to about 10° C.

3. The method of claim 1 or 2, wherein contacting the sample with the first aqueous medium comprises mixing the sample with the first aqueous medium.

4. The method of claim 3, wherein mixing the sample with the first aqueous medium comprises agitating the sample with the first aqueous medium.

5. The method of claim 4, wherein agitating is carried out by pipetting.

6. The method of claim 4, wherein agitating is carries out by vortexing or shaking.

7. The method of claim 4, wherein agitating is carried out by stirring.

8. The method of claim 4, wherein agitating is carried out with a paddle.

9. The method of claim 8, wherein the paddle is a USP apparatus 2.

10. The method of claim 1 or 2, wherein contacting the first WBC residual with the second aqueous medium comprises mixing the first WBC residual with the second aqueous medium.

11. The method of claim 10, wherein mixing the first WBC residual with the second aqueous medium comprises agitating the first WBC residual with the second aqueous medium.

12. The method of claim 11, wherein agitating is carried out by pipetting.

13. The method of claim 11, wherein agitating is carries out by vortexing or shaking.

14. The method of claim 11, wherein agitating is carried out by stirring.

15. The method of claim 11, wherein agitating is carried out with a paddle.

16. The method of claim 15, wherein the paddle is a USP apparatus 2.

17. The method of any one of claims 1-16, wherein the first nonionic surfactant and the second nonionic surfactant are the same.

18. The method of any one of claims 1-16, wherein the first nonionic surfactant and the second nonionic surfactant are different nonionic surfactants.

19. The method of any one of claims 1-18, wherein the first nonionic surfactant and the second nonionic surfactant are present in the same concentration.

20. The method of any one of claims 1-18, wherein the first nonionic surfactant and the second nonionic surfactant are present in different concentrations.

21. The method of any one of claims 1-20, further comprising measuring a concentration of an active form of the one or more NSPs of the first or second separated cell lysate.

22. The method of any one of claims 1-20, further comprising combining the first and second separated cell lysates to provide a first pooled cell lysate comprising a first pooled NSP extract, wherein the first pooled NSP extract comprises the one or more NSPs.

23. The method of claim 22, further comprising measuring a concentration of an active form of the one or more NSPs of the first pooled cell lysate comprising the first pooled NSP extract.

24. The method of any one of claims 1-16, further comprising:
contacting the second WBC residual with a third aqueous medium comprising at least 0.01% (v/v) of a third nonionic surfactant to obtain a third cell lysate comprising a third NSP extract, and a third WBC residual, wherein the third NSP extract comprises the one or more NSPs, and
separating the third cell lysate from the third WBC residual to provide a third separated cell lysate comprising the third NSP extract.

25. The method of claim 24, wherein contacting the second WBC residual with the third aqueous medium is performed at a temperature of from about 0° C. to about 10° C.

26. The method of claim 24 or 25, wherein contacting the second WBC residual with the third aqueous medium comprises mixing the second WBC residual with the third aqueous medium.

27. The method of claim 26, wherein mixing the second WBC residual with the third aqueous medium comprises agitating the second WBC residual with the third aqueous medium.

28. The method of claim 27, wherein agitating is carried out by pipetting.

29. The method of claim 27, wherein agitating is carries out by vortexing or shaking.

30. The method of claim 27, wherein agitating is carried out by stirring.

31. The method of claim 27, wherein agitating is carried out with a paddle.

32. The method of claim 31, wherein the paddle is a USP apparatus 2.

33. The method of any one of claims 24-32, wherein the first, second, and third nonionic surfactants are the same.

34. The method of any one of claims 24-32, wherein at least two of the first, second, and third nonionic surfactants are different nonionic surfactants.

35. The method of any one of claims 24-34, wherein the first, second, and third nonionic surfactants are present in the same concentration.

36. The method of any one of claims 24-34, wherein at least two of the first, second, and third nonionic surfactants are present in different concentrations.

37. The method of any one of claims 24-36, further comprising measuring a concentration of an active form of the one or more NSPs of the first, second, or third separated cell lysate.

38. The method of any one of claims 24-36, further comprising combining the third separated cell lysate with the first separated cell lysate, the second separated cell lysate, or the first and second separated cell lysates to provide a second pooled cell lysate comprising a second pooled NSP extract, wherein the second pooled NSP extract comprises the one or more NSPs.

39. The method of any one of claims 24-36, further comprising combining the third separated cell lysate with the first and second separated cell lysates to provide a second pooled cell lysate comprising a second pooled NSP extract, wherein the second pooled NSP extract comprises the one or more NSPs.

40. The method of claim 38 or 39, further comprising measuring a concentration of an active form of the one or more NSPs of the second pooled cell lysate comprising the second pooled NSP extract.

41. The method of any one of claims 24-32, further comprising:
contacting the third WBC residual with a fourth aqueous medium comprising at least 0.010% (v/v) of a fourth nonionic surfactant to obtain a fourth cell lysate comprising a fourth NSP extract, and a fourth WBC residual, wherein the fourth NSP extract comprises the one or more NSPs, and
separating the fourth cell lysate from the fourth WBC residual to provide a fourth separated cell lysate comprising the fourth NSP extract.

42. The method of claim 41, wherein contacting the third WBC residual with the fourth aqueous medium is performed at a temperature of from about 0° C. to about 10° C.

43. The method of claim 41 or 42, wherein contacting the third WBC residual with the fourth aqueous medium comprises mixing the third WBC residual with the fourth aqueous medium.

44. The method of claim 43, wherein mixing the third WBC residual with the fourth aqueous medium comprises agitating the third WBC residual with the fourth aqueous medium.

45. The method of claim 44, wherein agitating is carried out by pipetting.

46. The method of claim 44, wherein agitating is carries out by vortexing or shaking.

47. The method of claim 44, wherein agitating is carried out by stirring.

48. The method of claim 44, wherein agitating is carried out with a paddle.

49. The method of claim 48, wherein the paddle is a USP apparatus 2.

50. The method of any one of claims 41-49, wherein the first, second, third, and fourth nonionic surfactants are the same.

51. The method of any one of claims 41-49, wherein at least two of the first, second, third, and fourth nonionic surfactants are different nonionic surfactants.

52. The method of any one of claims 41-51, wherein the first, second, third, and fourth nonionic surfactants are present in the same concentration.

53. The method of any one of claims 41-51, wherein at least two of the first, second, third, and fourth nonionic surfactants are present in different concentrations.

54. The method of any one of claims 41-53, further comprising measuring a concentration of an active form of the one or more NSPs of the first, second, third, or fourth separated cell lysate.

55. The method of any one of claims 41-53, further comprising combining the fourth separated cell lysate with the first separated cell lysate, the second separated cell lysate, the third separated cell lysate, or a combination thereof to provide a third pooled cell lysate comprising a third pooled NSP extract, wherein the third pooled NSP extract comprises the one or more NSPs.

56. The method of any one of claims 41-53, further comprising combining the fourth separated cell lysate with the first, second, and third separated cell lysates to provide a third pooled cell lysate comprising a third pooled NSP extract, wherein the third pooled NSP extract comprises the one or more NSPs.

57. The method of claim 55 or 56, further comprising measuring a concentration of an active form of the one or more NSPs of the third pooled cell lysate comprising the third pooled NSP extract.

58. The method of any one of claims 41-49, further comprising:
contacting the fourth WBC residual with a fifth aqueous medium comprising at least 0.01% (v/v) of a fifth nonionic surfactant to obtain a fifth cell lysate comprising a fifth NSP extract, and a fifth WBC residual, wherein the fifth NSP extract comprises the one or more NSPs, and
separating the fifth cell lysate from the fifth WBC residual to provide a fifth separated cell lysate comprising the fifth NSP extract.

59. The method of claim 58, wherein contacting the fourth WBC residual with the fifth aqueous medium is performed at a temperature of from about 0° C. to about 10° C.

60. The method of claim 58 or 59, wherein contacting the fourth WBC residual with the fifth aqueous medium comprises mixing the fourth WBC residual with the fifth aqueous medium.

61. The method of claim 60, wherein mixing the fourth WBC residual with the fifth aqueous medium comprises agitating the fourth WBC residual with the fifth aqueous medium.

62. The method of claim 61, wherein agitating is carried out by pipetting.

63. The method of claim 61, wherein agitating is carries out by vortexing or shaking.

64. The method of claim 61, wherein agitating is carried out by stirring.

65. The method of claim 61, wherein agitating is carried out with a paddle.

66. The method of claim 65, wherein the paddle is a USP apparatus 2.

67. The method of any one of claims 58-66, wherein the first, second, third, fourth, and fifth nonionic surfactants are the same.

68. The method of any one of claims 58-66, wherein at least two of the first, second, third, fourth, and fifth nonionic surfactants are different nonionic surfactants.

69. The method of any one of claims 58-68, wherein the first, second, third, fourth, and fifth nonionic surfactants are present in the same concentration.

70. The method of any one of claims 58-68, wherein at least two of the first, second, third, fourth, and fifth nonionic surfactants are present in different concentrations.

71. The method of any one of claims 58-70, further comprising measuring a concentration of an active form of the one or more NSPs of the first, second, third, fourth, or fifth separated cell lysate.

72. The method of any one of claims 58-70, further comprising combining the fifth separated cell lysate with the first separated cell lysate, the second separated cell lysate, the third separated cell lysate, the fourth separated cell lysate, or a combination thereof to provide a fourth pooled cell lysate comprising a fourth pooled NSP extract, wherein the fourth pooled NSP extract comprises the one or more NSPs.

73. The method of any one of claims 58-70, further comprising combining the fifth separated cell lysate with the first, second, third, and fourth separated cell lysates to provide a fourth pooled cell lysate comprising a fourth pooled NSP extract, wherein the fourth pooled NSP extract comprises the one or more NSPs.

74. The method of claim 72 or 73, further comprising measuring a concentration of an active form of the one or more NSPs of the fourth pooled cell lysate comprising the fourth pooled NSP extract.

75. The method of any one of claims 58-66, further comprising,
contacting the fifth WBC residual with a sixth aqueous medium comprising at least 0.01% (v/v) of a sixth nonionic surfactant to obtain a sixth cell lysate comprising a sixth NSP extract, and a sixth WBC residual, wherein the sixth NSP extract comprises the one or more NSPs, and
separating the sixth cell lysate from the sixth WBC residual to provide a sixth separated cell lysate comprising the sixth NSP extract.

76. The method of claim 75, wherein contacting the fifth WBC residual with the sixth aqueous medium is performed at a temperature of from about 0° C. to about 10° C.

77. The method of claim 75 or 76, wherein contacting the fifth WBC residual with the sixth aqueous medium comprises mixing the fifth WBC residual with the sixth aqueous medium.

78. The method of claim 77, wherein mixing the fifth WBC residual with the sixth aqueous medium comprises agitating the fifth WBC residual with the sixth aqueous medium.

79. The method of claim 77, wherein agitating is carried out by pipetting.

80. The method of claim 77, wherein agitating is carries out by vortexing or shaking.

81. The method of claim 77, wherein agitating is carried out by stirring.

82. The method of claim 77, wherein agitating is carried out with a paddle.

83. The method of claim 82, wherein the paddle is a USP apparatus 2.

84. The method of any one of claims 75-83, wherein the first, second, third, fourth, fifth, and sixth nonionic surfactants are the same.

85. The method of any one of claims 75-83, wherein at least two of the first, second, third, fourth, fifth, and sixth nonionic surfactants are different nonionic surfactants.

86. The method of any one of claims 75-85, wherein the first, second, third, fourth, fifth, and sixth nonionic surfactants are present in the same concentration.

87. The method of any one of claims 75-85, wherein at least two of the first, second, third, fourth, fifth, and sixth nonionic surfactants are present in different concentrations.

88. The method of any one of claims 75-87, further comprising measuring a concentration of an active form of the one or more NSPs of the first, second, third, fourth, fifth, or sixth separated cell lysate.

89. The method of any one of claims 75-87, further comprising combining the sixth separated cell lysate with the first separated cell lysate, the second separated cell lysate, the third separated cell lysate, the fourth separated cell lysate, the fifth separated cell lysate, or a combination thereof to provide a fifth pooled cell lysate comprising a fifth pooled NSP extract, wherein the fifth pooled NSP extract comprises the one or more NSPs.

90. The method of any one of claims 75-87, further comprising combining the sixth separated cell lysate with the first, second, third, fourth, and fifth separated cell lysates to provide a fifth pooled cell lysate comprising a fifth pooled NSP extract, wherein the fifth pooled NSP extract comprises the one or more NSPs.

91. The method of claim 89 or 90, further comprising measuring a concentration of an active form of the one or more NSPs of the fifth pooled cell lysate comprising the fifth pooled NSP extract.

92. The method of any one of claims 1-91, wherein contacting the sample with a first aqueous medium comprises adding an aqueous wash solution to the sample to form a mixture of the aqueous wash solution and the sample, centrifuging the mixture of the aqueous wash solution and the sample to provide a supernatant and a pellet comprising the WBCs, collecting the supernatant, and contacting the pellet with the first aqueous medium.

93. The method of claim 92, wherein contacting the pellet with the first aqueous medium comprises mixing the pellet with the first aqueous medium.

94. The method of claim 93, wherein mixing the pellet with the first aqueous medium comprises agitating the pellet with the first aqueous medium.

95. The method of claim 94, wherein agitating is carried out by pipetting.

96. The method of claim 94, wherein agitating is carries out by vortexing or shaking.

97. The method of claim 94, wherein agitating is carried out by stirring.

98. The method of claim 94, wherein agitating is carried out with a paddle.

99. The method of claim 98, wherein the paddle is a USP apparatus 2.

100. The method of any one of claims 92-99, wherein the aqueous wash solution is a phosphate buffered saline solution, or a saline solution comprising about 0.9% NaCl.

101. The method of any one of claims 92-99, wherein the aqueous wash solution comprises a Tris-based alkaline buffer and NaCl.

102. The method of claim 101, wherein the aqueous wash solution comprises about 100 mM Tris and about 100 mM NaCl with a pH of about 7.5.

103. The method of any one of claims 92-102, wherein the supernatant comprises the one or more NSPs, and the method further comprises measuring a concentration of an active form of the one or more NSPs of the supernatant.

104. The method of any one of claims 1-103, wherein the first, second, third, fourth, fifth, or sixth aqueous medium, or a combination thereof comprises at least 0.02% (v/v) of the respective first, second, third, fourth, fifth, or sixth nonionic surfactant.

105. The method of claim 104, wherein the first or second aqueous medium, or a combination thereof comprises at least 0.02% (v/v) of the respective first or second nonionic surfactant.

106. The method of any one of claims 1-104, wherein the first, second, third, fourth, fifth, or sixth aqueous medium, or a combination thereof comprises at least 0.05% (v/v) of the respective first, second, third, fourth, fifth, or sixth nonionic surfactant.

107. The method of claim 106, wherein the first or second aqueous medium, or a combination thereof comprises at least 0.05% (v/v) of the respective first or second nonionic surfactant.

108. The method of any one of claims 1-104, wherein the first, second, third, fourth, fifth, or sixth aqueous medium, or a combination thereof comprises from about 0.02% (v/v) to about 1.5% (v/v) of the respective first, second, third, fourth, fifth, or sixth nonionic surfactant.

109. The method of claim 108, wherein the first or second aqueous medium, or a combination thereof comprises from about 0.02% (v/v) to about 1.5% (v/v) of the respective first or second nonionic surfactant.

110. The method of claim 108, wherein the first, second, third, fourth, fifth, or sixth aqueous medium, or a combination thereof comprises from about 0.03% (v/v) to about 1% (v/v) of the respective first, second, third, fourth, fifth, or sixth nonionic surfactant.

111. The method of claim 110, wherein the first or second aqueous medium, or a combination thereof comprises from about 0.03% (v/v) to about 1% (v/v) of the respective first or second nonionic surfactant.

112. The method of claim 110, wherein the first, second, third, fourth, fifth, or sixth aqueous medium, or a combination thereof comprises from about 0.04% (v/v) to about 0.8% (v/v) of the respective first, second, third, fourth, fifth, or sixth nonionic surfactant.

113. The method of claim 112, wherein the first or second aqueous medium, or a combination thereof comprises from about 0.04% (v/v) to about 0.8% (v/v) of the respective first or second nonionic surfactant.

114. The method of claim 112, wherein the first, second, third, fourth, fifth, or sixth aqueous medium, or a combination thereof comprises from about 0.05% (v/v) to about 0.6% (v/v) of the respective first, second, third, fourth, fifth, or sixth nonionic surfactant.

115. The method of claim 114, wherein the first or second aqueous medium, or a combination thereof comprises from about 0.05% (v/v) to about 0.6% (v/v) of the respective first or second nonionic surfactant.

116. The method of claim 114, wherein the first, second, third, fourth, fifth, or sixth aqueous medium, or a combination thereof comprises about 0.05% (v/v) of the respective first, second, third, fourth, fifth, or sixth nonionic surfactant.

117. The method of claim 116, wherein the first or second aqueous medium, or a combination thereof comprises about 0.05% (v/v) of the respective first or second nonionic surfactant.

118. The method of any one of claims 1-117, wherein the first, second, third, fourth, fifth, or sixth nonionic surfactant, or a combination thereof, is a nonionic polyoxyethylene surfactant.

119. The method of any one of claims 1-117, wherein the first nonionic surfactant is a nonionic polyoxyethylene surfactant.

120. The method of any one of claims 1-117, wherein the second nonionic surfactant is a nonionic polyoxyethylene surfactant.

121. The method of any one of claims 24-117, wherein the third nonionic surfactant is a nonionic polyoxyethylene surfactant.

122. The method of any one of claims 41-117, wherein the fourth nonionic surfactant is a nonionic polyoxyethylene surfactant.

123. The method of any one of claims 58-117, wherein the fifth nonionic surfactant is a nonionic polyoxyethylene surfactant.

124. The method of any one of claims 75-117, wherein the sixth nonionic surfactant is a nonionic polyoxyethylene surfactant.

125. The method of any one of claims 118-124, wherein the first, second, third, fourth, fifth, or sixth nonionic surfactant, or a combination thereof is a nonionic polyoxyethylene surfactant selected from the group consisting of octylphenoxypolyethoxyethanol, 2-[4-(2,4,4-trimethylpentan-2-yl)phenoxy]ethanol, polyoxyethylene nonylphenylether (branched), and polyethylene glycol sorbitan monolaurate.

126. The method of claim 125, wherein the first, second, third, fourth, fifth, or sixth nonionic surfactant, or a combination thereof is octylphenoxypolyethoxyethanol.

127. The method of claim 125 or 126, wherein the first nonionic surfactant is octylphenoxypolyethoxyethanol.

128. The method of claim 125 or 126, wherein the second nonionic surfactant is octylphenoxypolyethoxyethanol.

129. The method of claim 125 or 126, wherein the third nonionic surfactant is octylphenoxypolyethoxyethanol.

130. The method of claim 125 or 126, wherein the fourth nonionic surfactant is octylphenoxypolyethoxyethanol.

131. The method of claim 125 or 126, wherein the fifth nonionic surfactant is octylphenoxypolyethoxyethanol.

132. The method of claim 125 or 126, wherein the sixth nonionic surfactant is octylphenoxypolyethoxyethanol.

133. The method of any one of claims 126-132, wherein the first, second, third, fourth, fifth, or sixth aqueous medium, or a combination thereof comprises about 0.05% (v/v) of octylphenoxypolyethoxyethanol, about 0.75 M NaCl, and about 50 mM HEPES.

134. The method of claim 133, wherein the first or second aqueous medium, or a combination thereof comprises about 0.05% (v/v) of octylphenoxypolyethoxyethanol, about 0.75 M NaCl, and about 50 mM HEPES.

135. The method of any one of claims 1-134, wherein the one or more NSPs comprise neutrophil elastase (NE), proteinase 3 (PR3), cathepsin G (CatG), neutrophil serine protease 4 (NSP4), or a combination thereof.

136. The method of claim 135, wherein the one or more NSPs comprise NE.

137. The method of claim 135 or 136, wherein the one or more NSPs comprise PR3.

138. The method of any one of claims 135-137, wherein the one or more NSPs comprise CatG.

139. The method of any one of claims 135-138, wherein the one or more NSPs comprise NSP4.

140. The method of any one of claims 1-139, wherein the subject is a human subject.

141. A method of treating a DPP1-mediated condition in a patient in need thereof, comprising:
(a) measuring a baseline concentration of an active form of one or more NSPs extracted from a first sample comprising white blood cells obtained from the patient,
(b) orally administering to the patient daily for a first administration period of about 2 weeks to about 16 weeks, a pharmaceutical composition comprising a first daily dosage of about 10 mg to about 40 mg of a compound of formula (I), or a pharmaceutically acceptable salt thereof, 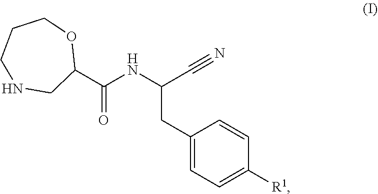
wherein,
R¹ is 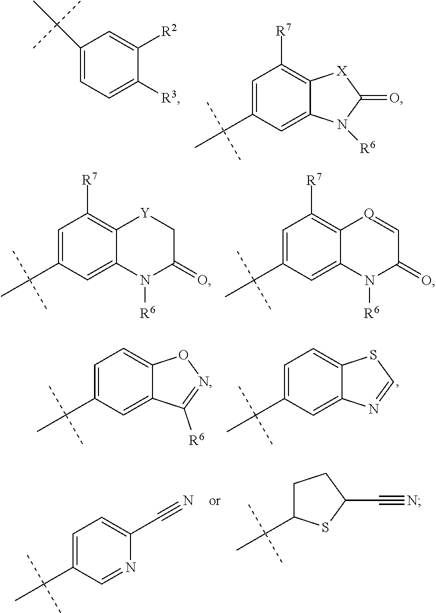
R² is hydrogen, F, Cl, Br, OSO₂C₁₋₃alkyl, or C₁₋₃alkyl;
R³ is hydrogen, F, Cl, Br, CN, CF₃, SO₂C₁₋₃alkyl, CONH₂ or SO₂NR⁴R⁵, wherein R⁴ and R⁵ together with the nitrogen atom to which they are attached form an azetidine, pyrrolidine or piperidine ring;
X is O, S or CF₂;
Y is O or S;
Q is CH or N;
R⁶ is C₁₋₃alkyl, wherein the C₁₋₃alkyl is optionally substituted by 1, 2 or 3 F and optionally by one substituent selected from OH, OC₁₋₃alkyl, N(C₁₋₃alkyl)₂, cyclopropyl, or tetrahydropyran; and
R⁷ is hydrogen, F, Cl or CH₃;
(c) measuring a concentration of the active form of the one or more NSPs extracted from a second sample comprising white blood cells, wherein the second sample is obtained from the patient during the first administration period, or about one week or less subsequent to the first administration period,
(d) comparing the concentration from the second sample with the baseline concentration from the first sample; and
if the concentration from the second sample is reduced by about 10% or more as compared to the baseline concentration from the first sample, then orally administering to the patient daily for a second administration period the same daily dosage as the first daily dosage of the compound of formula (I), or a pharmaceutically acceptable salt thereof, or
if the concentration from the second sample is not reduced by about 10% or more as compared to the baseline concentration from the first sample, then orally administering to the patient daily for a second administration period a second daily dosage of the compound of formula (I), or a pharmaceutically acceptable salt thereof, wherein the second daily dosage is about 1.5 times to about 7 times the first daily dosage.

142. The method of claim 141, wherein the compound of formula (I), or a pharmaceutically acceptable salt thereof, is the S,S diastereomer:

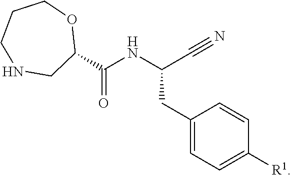

143. The method of claim 141, wherein the compound of formula (I), or a pharmaceutically acceptable salt thereof, is the S,R diastereomer:

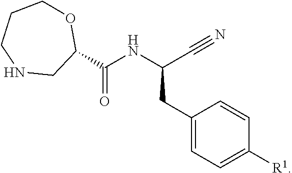

144. The method of claim 141, wherein the compound of formula (I), or a pharmaceutically acceptable salt thereof, is the R,S diastereomer:

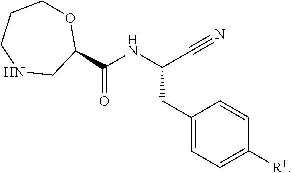

145. The method of claim 141, wherein the compound of formula (I), or a pharmaceutically acceptable salt thereof, is the R,R diastereomer:

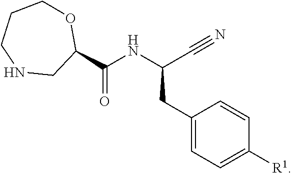

146. The method of claim 141, wherein the composition comprises a mixture of an S,S diastereomer of a compound of formula (I) and an S,R diastereomer of a compound of formula (I).

147. The method of claim 141, wherein the composition comprises a mixture of an S,S diastereomer of a compound of formula (I) and an R,S diastereomer of a compound of formula (I).

148. The method of claim 141, wherein the composition comprises a mixture of an S,S diastereomer of a compound of formula (I) and an R,R diastereomer of a compound of formula (I).

149. The method of any one of claims 141-148, wherein R¹ is 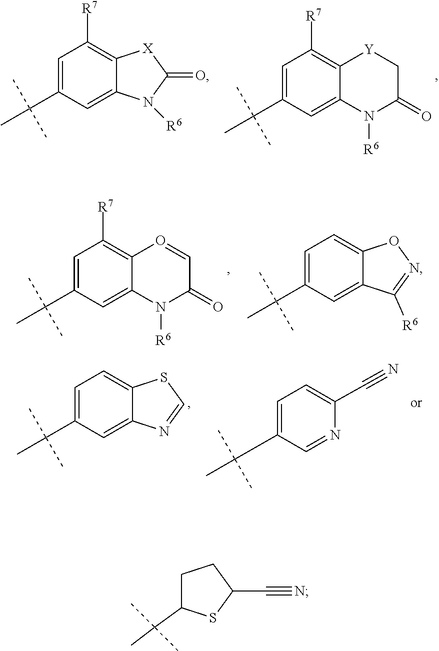
X is O, S or CF₂;
Y is O or S;
Q is CH or N;
R⁶ is C₁₋₃alkyl, wherein the C₁₋₃alkyl is optionally substituted by 1, 2 or 3 F and optionally by one substituent selected from OH, OC₁₋₃alkyl, N(C₁₋₃alkyl)₂, cyclopropyl, or tetrahydropyran; and
R⁷ is hydrogen, F, Cl or CH₃.

150. The method of any one of claims 141-149, wherein,
R₁ is 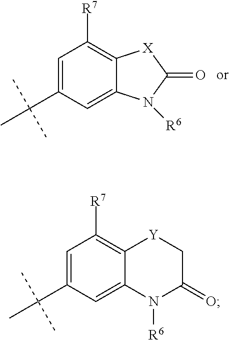
X is O, S or CF₂;
Y is O or S;
R⁶ is C₁₋₃alkyl, wherein the C₁₋₃alkyl is optionally substituted by 1, 2 or 3 F and optionally by one substituent selected from OH, OC₁₋₃alkyl, N(C₁₋₃alkyl)₂, cyclopropyl, or tetrahydropyran; and
R⁷ is hydrogen, F, Cl or CH₃.

151. The method of any one of claims 141-150, wherein, R¹ is

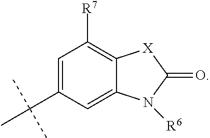

152. The method of any one of claims 141-151, wherein X is O, S or CF₂; R⁶ is C₁₋₃alkyl, wherein the C₁₋₃alkyl is optionally substituted by 1, 2 or 3 F; and R⁷ is hydrogen, F, Cl or CH₃.

153. The method of any one of claims 141-151, wherein X is O; R⁶ is C₁₋₃alkyl, wherein the C₁₋₃alkyl is optionally substituted by 1, 2 or 3 F; and R⁷ is hydrogen.

154. The method of any one of claims 141-151, wherein X is O; R⁶ is C₁₋₃alkyl; and R⁷ is hydrogen.

155. The method of claim 141 or 142, wherein the compound of formula (I) is selected from the group consisting of
(2S)—N-[(1S)-1-Cyano-2-(4′-cyanobiphenyl-4-yl)ethyl]-1,4-oxazepane-2-carboxamide;
(2S)—N-{(1S)-1-Cyano-2-[4-(3-methyl-2-oxo-2,3-dihydro-1,3-benzoxazol-5-yl)phenyl]ethyl}-1,4-oxazepane-2-carboxamide;
(2S)—N-{(1S)-1-Cyano-2-[4-(3,7-dimethyl-2-oxo-2,3-dihydro-1,3-benzoxazol-5-yl)phenyl]ethyl}-1,4-oxazepane-2-carboxamide;
4′-[(2S)-2-Cyano-2-{[(2S)-1,4-oxazepan-2-ylcarbonyl]amino}ethyl]biphenyl-3-yl methanesulfonate;
(2S)—N-{(1S)-1-Cyano-2-[4-(3-methyl-1,2-benzoxazol-5-yl)phenyl]ethyl}-1,4-oxazepane-2-carboxamide;
(2S)—N-{(1S)-1-Cyano-2-[4′-(trifluoromethyl)biphenyl-4-yl]ethyl}-1,4-oxazepane-2-carboxamide;
(2S)—N-[(1S)-1-Cyano-2-(3′,4′-difluorobiphenyl-4-yl)ethyl]-1,4-oxazepane-2-carboxamide;
(2S)—N-{(1S)-1-Cyano-2-[4-(6-cyanopyridin-3-yl)phenyl]ethyl}-1,4-oxazepane-2-carboxamide;
(2S)—N-{(1S)-1-Cyano-2-[4-(4-methyl-3-oxo-3,4-dihydro-2H-1,4-benzothiazin-6-yl)phenyl]ethyl}-1,4-oxazepane-2-carboxamide;
(2S)—N-{(1S)-1-Cyano-2-[4-(3-ethyl-7-methyl-2-oxo-2,3-dihydro-1,3-benzoxazol-5-yl)phenyl]ethyl}-1,4-oxazepane-2-carboxamide;
(2S)—N-[(1S)-1-Cyano-2-{4-[3-(2-hydroxy-2-methylpropyl)-2-oxo-2,3-dihydro-1,3-benzoxazol-5-yl]phenyl}ethyl]-1,4-oxazepane-2-carboxamide;
(2S)—N-[(1S)-1-Cyano-2-{4-[3-(2,2-difluoroethyl)-7-fluoro-2-oxo-2,3-dihydro-1,3-benzoxazol-5-yl]phenyl}ethyl]-1,4-oxazepane-2-carboxamide;
(2S)—N-[(1S)-1-Cyano-2-(4-{3-[2-(dimethylamino)ethyl]-2-oxo-2,3-dihydro-1,3-benzoxazol-5-yl}phenyl)ethyl]-1,4-oxazepane-2-carboxamide;
(2S)—N-{(1S)-1-Cyano-2-[4-(3,3-difluoro-1-methyl-2-oxo-2,3-dihydro-1H-indol-6-yl)phenyl]ethyl}-1,4-oxazepane-2-carboxamide;
(2S)—N-{(1S)-1-Cyano-2-[4-(7-fluoro-3-methyl-2-oxo-2,3-dihydro-1,3-benzoxazol-5-yl)phenyl]ethyl}-1,4-oxazepane-2-carboxamide;
(2S)—N-{(1S)-1-Cyano-2-[4-(3-ethyl-2-oxo-2,3-dihydro-1,3-benzoxazol-5-yl)phenyl]ethyl}-1,4-oxazepane-2-carboxamide;
(2S)—N-[(1S)-1-Cyano-2-{4-[3-(cyclopropylmethyl)-2-oxo-2,3-dihydro-1,3-benzoxazol-5-yl]phenyl}ethyl]-1,4-oxazepane-2-carboxamide;
(2S)—N-[(1S)-1-Cyano-2-{4-[3-(2-methoxyethyl)-2-oxo-2,3-dihydro-1,3-benzothiazol-5-yl]phenyl}ethyl]-1,4-oxazepane-2-carboxamide;
(2S)—N-[(1S)-1-Cyano-2-{4-[2-oxo-3-(propan-2-yl)-2,3-dihydro-1,3-benzoxazol-5-yl]phenyl}ethyl]-1,4-oxazepane-2-carboxamide;
(2S)—N-{(1S)-1-Cyano-2-[4-(4-methyl-3-oxo-3,4-dihydro-2H-1,4-benzoxazin-6-yl)phenyl]ethyl}-1,4-oxazepane-2-carboxamide;
(2S)—N-[(1S)-1-Cyano-2-{4-[3-(2-methoxyethyl)-2-oxo-2,3-dihydro-1,3-benzoxazol-5-yl]phenyl}ethyl]-1,4-oxazepane-2-carboxamide;
(2S)—N-{(1S)-1-Cyano-2-[4-(5-cyanothiophen-2-yl)phenyl]ethyl}-1,4-oxazepane-2-carboxamide;
(2S)—N-[(1S)-2-(4′-Carbamoyl-3′-fluorobiphenyl-4-yl)-1-cyanoethyl]-1,4-oxazepane-2-carboxamide;
(2S)—N-{(1S)-1-Cyano-2-[4-(1-methyl-2-oxo-1,2-dihydroquinolin-7-yl)phenyl]ethyl}-1,4-oxazepane-2-carboxamide;
(2S)—N-[(1S)-1-Cyano-2-{4-[2-oxo-3-(tetrahydro-2H-pyran-4-ylmethyl)-2,3-dihydro-1,3-benzoxazol-5-yl]phenyl}ethyl]-1,4-oxazepane-2-carboxamide;
(2S)—N-{(1S)-2-[4-(7-Chloro-3-methyl-2-oxo-2,3-dihydro-1,3-benzoxazol-5-yl)phenyl]-1-cyanoethyl}-1,4-oxazepane-2-carboxamide;
(2S)—N-[(1S)-1-Cyano-2-{4-[3-(2,2-difluoroethyl)-2-oxo-2,3-dihydro-1,3-benzoxazol-5-yl]phenyl}ethyl]-1,4-oxazepane-2-carboxamide;
(2S)—N-[(1S)-1-Cyano-2-{4-[2-oxo-3-(2,2,2-trifluoroethyl)-2,3-dihydro-1,3-benzoxazol-5-yl]phenyl}ethyl]-1,4-oxazepane-2-carboxamide;
(2S)—N-{(1S)-1-Cyano-2-[4-(3-methyl-2-oxo-2,3-dihydro-1,3-benzothiazol-5-yl)phenyl]ethyl}-1,4-oxazepane-2-carboxamide;
(2S)—N-{(1S)-1-Cyano-2-[4′-(methylsulfonyl)biphenyl-4-yl]ethyl}-1,4-oxazepane-2-carboxamide;
(2S)—N-{(1S)-2-[4′-(Azetidin-1-ylsulfonyl)biphenyl-4-yl]-1-cyanoethyl}-1,4-oxazepane-2-carboxamide;
(2S)—N-[(1S)-1-Cyano-2-(4′-fluorobiphenyl-4-yl)ethyl]-1,4-oxazepane-2-carboxamide;
(2S)—N-{(1S)-2-[4-(1,3-Benzothiazol-5-yl)phenyl]-1-cyanoethyl}-1,4-oxazepane-2-carboxamide;
(2S)—N-[(1S)-1-Cyano-2-(4′-cyanobiphenyl-4-yl)ethyl]-1,4-oxazepane-2-carboxamide;
and pharmaceutically acceptable salts thereof.

156. The method of claim 141 or 142, wherein the compound of formula (I) is brensocatib; or a pharmaceutically acceptable salt thereof.

157. The method of claim 141 or 142, wherein the compound of formula (I) is brensocatib.

158. The method of claim 141 or 143, wherein the compound of formula (I) is (2S)—N-{(1R)-1-Cyano-2-[4-(3-methyl-2-oxo-2,3-dihydro-1,3-benzoxazol-5-yl)phenyl]ethyl}-1,4-oxazepane-2-carboxamide: 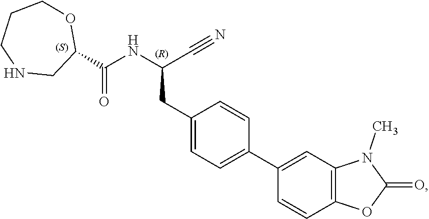
or a pharmaceutically acceptable salt thereof.

159. The method of claim 158, wherein the compound of formula (I) is (2S)—N-{(1R)-1-Cyano-2-[4-(3-methyl-2-oxo-2,3-dihydro-1,3-benzoxazol-5-yl)phenyl]ethyl}-1,4-oxazepane-2-carboxamide:

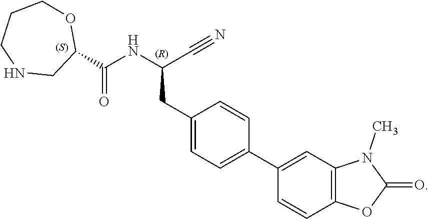

160. The method of claim 141 or 144, wherein the compound of formula (I) is (2R)—N-{(1S)-1-Cyano-2-[4-(3-methyl-2-oxo-2,3-dihydro-1,3-benzoxazol-5-yl)phenyl]ethyl}-1,4-oxazepane-2-carboxamide: 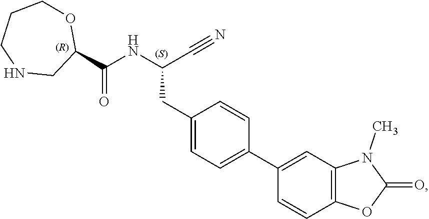
or a pharmaceutically acceptable salt thereof.

161. The method of claim 160, wherein the compound of formula (I) is (2R)—N-{(1S)-1-Cyano-2-[4-(3-methyl-2-oxo-2,3-dihydro-1,3-benzoxazol-5-yl)phenyl]ethyl}-1,4-oxazepane-2-carboxamide:

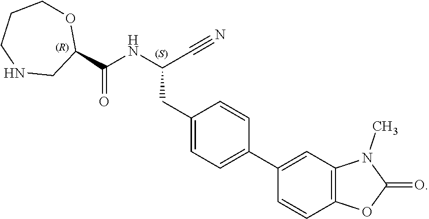

162. The method of claim 141 or 145, wherein the compound of formula (I) is (2R)—N-{(1R)-1-Cyano-2-[4-(3-methyl-2-oxo-2,3-dihydro-1,3-benzoxazol-5-yl)phenyl]ethyl}-1,4-oxazepane-2-carboxamide: 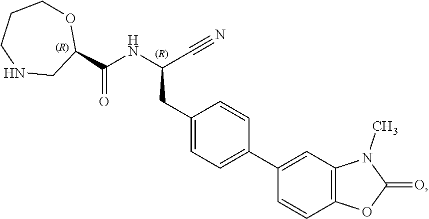
or a pharmaceutically acceptable salt thereof.

163. The method of claim 162, wherein the compound of formula (I) is (2R)—N-{(1R)-1-Cyano-2-[4-(3-methyl-2-oxo-2,3-dihydro-1,3-benzoxazol-5-yl)phenyl]ethyl}-1,4-oxazepane-2-carboxamide:

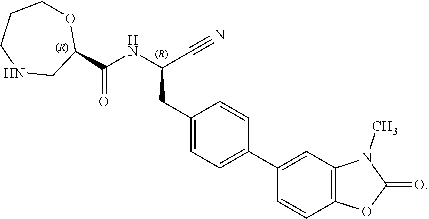

164. The method of claim 141, wherein the composition comprises a mixture of brensocatib, or a pharmaceutically acceptable salt thereof, and (2S)—N-{(1R)-1-Cyano-2-[4-(3-methyl-2-oxo-2,3-dihydro-1,3-benzoxazol-5-yl)phenyl]ethyl}-1,4-oxazepane-2-carboxamide: 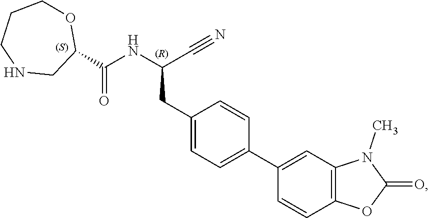
or a pharmaceutically acceptable salt thereof.

165. The method of claim 141, wherein the composition comprises a mixture of brensocatib, or a pharmaceutically acceptable salt thereof, and (2R)—N-{(1S)-1-Cyano-2-[4-(3-methyl-2-oxo-2,3-dihydro-1,3-benzoxazol-5-yl)phenyl]ethyl}-1,4-oxazepane-2-carboxamide: 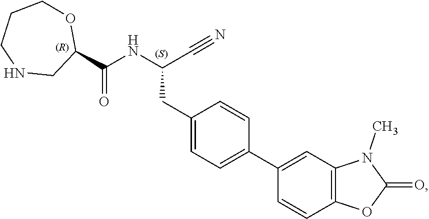
or a pharmaceutically acceptable salt thereof.

166. The method of claim 141, wherein the composition comprises a mixture of brensocatib, or a pharmaceutically acceptable salt thereof, and (2R)—N-{(1R)-1-Cyano-2-[4-(3-methyl-2-oxo-2,3-dihydro-1,3-benzoxazol-5-yl)phenyl]ethyl}-1,4-oxazepane-2-carboxamide: 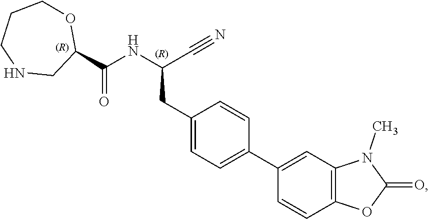
or a pharmaceutically acceptable salt thereof.

167. The method of any one of claims 141-166, wherein the composition comprises a pharmaceutically acceptable adjuvant, diluent or carrier.

168. The method of any one of claims 141-167, wherein the composition comprises:
(a) from about 1 to about 30 wt % of the compound of formula (I), or a pharmaceutically acceptable salt thereof,
(b) from about 55 to about 75 wt % of a pharmaceutical diluent,
(c) from about 15 to about 25 wt % of a compression aid,
(d) from about 3 to about 5 wt % of a pharmaceutical disintegrant,
(e) from about 0.00 to about 1 wt % of a pharmaceutical glidant; and
(f) from about 2 to about 6 wt % of a pharmaceutical lubricant,
wherein the component weights add up to 100 wt %.

169. The method of claim 168, wherein the pharmaceutical lubricant is glycerol behenate.

170. The method of claim 168 or 169, wherein the pharmaceutical diluent is microcrystalline cellulose.

171. The method of any one of claims 168-170, wherein the compression aid is dibasic calcium phosphate dihydrate.

172. The method of any one of claims 168-171, wherein the pharmaceutical disintegrant is sodium starch glycolate.

173. The method of any one of claims 168-172, wherein the pharmaceutical glidant is silicon dioxide.

174. The method of any one of claims 168-173, wherein the composition is in tablet form.

175. The method of claim 174, wherein the composition further comprises a tablet coating.

176. The method of any one of claims 168-175, wherein the compound of formula (I) is present at about 3 to about 10 wt % of the total weight of the pharmaceutical composition.

177. The method of claim 176, wherein the pharmaceutical lubricant is glycerol behenate and the glycerol behenate is present at about 2.5 to about 4.5 wt % of the total weight of the composition.

178. The method of claim 176 or 177, wherein the pharmaceutical glidant is silicon dioxide and the silicon dioxide is present at about 0.05 to about 0.25 wt % of the total weight of the composition.

179. The method of any one of claims 176-178, wherein the pharmaceutical disintegrant is sodium starch glycolate and the sodium starch glycolate is present at about 3.5 to about 4.5 wt % of the total weight of the composition.

180. The method of any one of claims 176-179, wherein the compression aid is dibasic calcium phosphate dihydrate and the dibasic calcium phosphate dihydrate is present at about 18 to about 22 wt % of the total weight of the composition.

181. The method of any one of claims 176-180, wherein the pharmaceutical diluent is microcrystalline cellulose and the microcrystalline cellulose is present at about 55 to about 70 wt % of the total weight of the composition.

182. The method of any one of claims 141-181, wherein the second daily dosage is about 1.5 times to about 6 times the first daily dosage.

183. The method of claim 182, wherein the second daily dosage is about 1.5 times to about 5 times the first daily dosage.

184. The method of claim 182, wherein the second daily dosage is about 1.5 times to about 4 times the first daily dosage.

185. The method of claim 182, wherein the second daily dosage is about 1.5 times to about 3 times the first daily dosage.

186. The method of claim 182, wherein the second daily dosage is about 1.5 times to about 2 times the first daily dosage.

187. The method any one of claims 141-186, wherein the first daily dosage of the compound of formula (I), or a pharmaceutically acceptable salt thereof is about 10 mg to about 25 mg.

188. The method any one of claims 141-187, wherein the first daily dosage of the compound of formula (I), or a pharmaceutically acceptable salt thereof is about 10 mg to about 15 mg.

189. The method of any one of claims 141-188, wherein the first daily dosage of the compound of formula (I), or a pharmaceutically acceptable salt thereof is about 10 mg to about 12 mg.

190. The method of any one of claims 184-186, wherein the first daily dosage of the compound of formula (I), or a pharmaceutically acceptable salt thereof is about 16 mg to about 25 mg.

191. The method of claim 185 or 186, wherein the first daily dosage of the compound of formula (I), or a pharmaceutically acceptable salt thereof is about 20 mg to about 25 mg.

192. The method of claim 185 or 186, wherein the first daily dosage of the compound of formula (I), or a pharmaceutically acceptable salt thereof is about 25 mg to about 40 mg.

193. The method of any one of claims 141-181, wherein the first daily dosage of the compound of formula (I), or a pharmaceutically acceptable salt thereof is about 10 mg, and the second daily dosage is about 2 times to about 6.5 times the first daily dosage.

194. The method of any one of claims 141-181, wherein the first daily dosage of the compound of formula (I), or a pharmaceutically acceptable salt thereof is about 25 mg, and the second daily dosage is about 1.6 times to about 2.6 times the first daily dosage.

195. The method of any one of claims 141-194, wherein the second sample is obtained from the patient during the first administration period.

196. The method of claim 195, wherein the second sample is obtained from the patient at the end of the first administration period.

197. The method of claim 195, wherein the second sample is obtained from the patient about seven days before the end of the first administration period.

198. The method of claim 195, wherein the second sample is obtained from the patient about six days before the end of the first administration period.

199. The method of claim 195, wherein the second sample is obtained from the patient about five days before the end of the first administration period.

200. The method of claim 195, wherein the second sample is obtained from the patient about four days before the end of the first administration period.

201. The method of claim 195, wherein the second sample is obtained from the patient about three days before the end of the first administration period.

202. The method of claim 195, wherein the second sample is obtained from the patient about two days before the end of the first administration period.

203. The method of claim 195, wherein the second sample is obtained from the patient about one day before the end of the first administration period.

204. The method of any one of claims 141-194, wherein the second sample is obtained from the patient about one week subsequent to the first administration period.

205. The method of any one of claims 141-194, wherein the second sample is obtained from the patient about one day subsequent to the first administration period.

206. The method of any one of claims 141-194, wherein the second sample is obtained from the patient about two days subsequent to the first administration period.

207. The method of any one of claims 141-194, wherein the second sample is obtained from the patient about three days subsequent to the first administration period.

208. The method of any one of claims 141-194, wherein the second sample is obtained from the patient about four days subsequent to the first administration period.

209. The method of any one of claims 141-194, wherein the second sample is obtained from the patient about five days subsequent to the first administration period.

210. The method of any one of claims 141-194, wherein the second sample is obtained from the patient about six days subsequent to the first administration period.

211. The method of any one of claims 141-194, wherein the second sample is obtained from the patient about seven days subsequent to the first administration period.

212. The method of any one of claims 141-211, wherein the first administration period is about 2 weeks to about 12 weeks.

213. The method of any one of claims 141-212, wherein the first administration period is about 2 weeks to about 8 weeks.

214. The method of any one of claims 141-213, wherein the first administration period is about 3 weeks to about 6 weeks.

215. The method of any one of claims 141-214, wherein the first administration period is about 3 weeks to about 5 weeks.

216. The method of claim 212, wherein the first administration period is about three weeks.

217. The method of claim 212, wherein the first administration period is about four weeks.

218. The method of claim 212, wherein the first administration period is about five weeks.

219. The method of claim 212, wherein the first administration period is about 6 weeks.

220. The method of claim 212, wherein the first administration period is about 7 weeks.

221. The method of claim 212, wherein the first administration period is about 8 weeks.

222. The method of claim 212, wherein the first administration period is about 9 weeks.

223. The method of claim 212, wherein the first administration period is about 10 weeks.

224. The method of claim 212, wherein the first administration period is about 11 weeks.

225. The method of claim 212, wherein the first administration period is about 12 weeks.

226. The method of any one of claims 141-195, wherein the first administration period is about 4 weeks, and the second sample is obtained from the patient at about 4 weeks during the first administration period.

227. The method of any one of claims 141-226, wherein the one or more NSPs comprise NE.

228. The method of claim 227, wherein if the concentration of the active form of NE from the second sample is reduced by about 19% or more as compared to the baseline concentration of the active form of NE from the first sample, then orally administering daily for the second administration period the same daily dosage as the first daily dosage of the compound of formula (I), or a pharmaceutically acceptable salt thereof, or if the concentration of the active form of NE from the second sample is not reduced by about 19% or more as compared to the baseline concentration of the active form of NE from the first sample, then orally administering daily for the second administration period the second daily dosage of the compound of formula (I), or a pharmaceutically acceptable salt thereof.

229. The method of any one of claims 141-228, wherein the one or more NSPs comprise PR3.

230. The method of any one of claims 141-229, wherein the one or more NSPs comprise CatG.

231. The method of any one of claims 141-230, wherein the one or more NSPs comprise NSP4.

232. The method of any one of claims 141-231, wherein the second administration period is at least 1 month.

233. The method of any one of claims 141-232, wherein the second administration period is from about 1 month to about 12 months.

234. The method of any one of claims 141-232, wherein the second administration period is from about 5 months to about 24 months.

235. The method of any one of claims 141-232, wherein the second administration period is from about 5 months to about 18 months.

236. The method of any one of claims 141-232, wherein the second administration period is from about 5 months to about 15 months.

237. The method of any one of claims 141-232, wherein the second administration period is from about 3 months to about 6 months.

238. The method of any one of claims 141-232, wherein the second administration period is from about 6 months to about 12 months.

239. The method of any one of claims 141-232, wherein the second administration period is from about 12 months to about 18 months.

240. The method of any one of claims 141-232, wherein the second administration period is from about 12 months to about 24 months.

241. The method of any one of claims 141-240, wherein orally administering to the patient daily during the first and second administration periods is carried out one time daily.

242. The method of any one of claims 141-240, wherein orally administering to the patient daily during the first and second administration periods is carried out two times daily.

243. The method of any one of claims 141-242, wherein the one or more NSPs are extracted from the first sample by the method of any one of claims 1-140.

244. The method of any one of claims 141-243, wherein the one or more NSPs are extracted from the second sample by the method of any one of claims 1-140.

245. The method of any one of claims 141-244, wherein the DPP1-mediated condition is an obstructive disease of the airways.

246. The method of claim 245, wherein the obstructive disease of the airways is asthma, chronic obstructive pulmonary disease (COPD), bronchitis, emphysema, bronchiectasis, cystic fibrosis, sarcoidosis, alpha-1 antitrypsin deficiency, farmer's lung or a related disease, hypersensitivity pneumonitis, lung fibrosis, acute or chronic rhinitis, perennial and seasonal allergic rhinitis, nasal polyposis, acute respiratory distress syndrome (ARDS), or an obstructive disease of the airways due to a respiratory syncytial virus, influenza, coronavirus or adenovirus infection.

247. The method of claim 246, wherein the obstructive disease of the airways is bronchiectasis.

248. The method of claim 247, wherein the bronchiectasis is non-cystic fibrosis bronchiectasis.

249. The method of claim 246, wherein the obstructive disease of the airways is cystic fibrosis.

250. The method of claim 246, wherein the obstructive disease of the airways is alpha-1 antitrypsin deficiency.

251. The method of claim 246, wherein the obstructive disease of the airways is COPD.

252. The method of claim 246, wherein the obstructive disease of the airways is asthma.

253. The method of claim 252, wherein the asthma is bronchial, allergic, intrinsic, extrinsic or dust asthma.

254. The method of claim 246, wherein the obstructive disease of the airways is acute respiratory distress syndrome (ARDS).

255. The method of any one of claims 141-244, wherein the DPP1-mediated condition is an antineutrophil cytoplasmic autoantibody (ANCA) associated vasculitis.

256. The method of claim 255, wherein the ANCA associated vasculitis is granulomatosis with polyangiitis (GPA).

257. The method of claim 255, wherein the ANCA associated vasculitis is microscopic polyangiitis (MPA).

258. The method of any one of claims 141-244, wherein the DPP1-mediated condition is cancer.

259. The method of claim 258, wherein the cancer is a primary solid tumor, a liquid tumor, or a metastatic cancer.

260. The method of claim 259, wherein the DPP1 is expressed by cancerous cells, neutrophils, macrophages, monocytes, or mast cells.

261. The method of claim 259 or 260, wherein the cancer is a metastatic cancer.

262. The method of claim 261, wherein the metastatic cancer comprises metastatic breast cancer.

263. The method of claim 262, wherein the metastatic breast cancer comprises metastasis of breast cancer to the lung, brain, bone, pancreas, lymph nodes, and/or liver.

264. The method of claim 263, wherein the metastatic breast cancer comprises metastasis of breast cancer to the lung.

265. The method of claim 263, wherein the metastatic breast cancer comprises metastasis of breast cancer to the brain.

266. The method of claim 263, wherein the metastatic breast cancer comprises metastasis of breast cancer to the bone.

267. The method of claim 263, wherein the metastatic breast cancer comprises metastasis of breast cancer to the pancreas.

268. The method of claim 263, wherein the metastatic breast cancer comprises metastasis of breast cancer to the lymph nodes.

269. The method of claim 263, wherein the metastatic breast cancer comprises metastasis of breast cancer to the liver.

270. The method of claim 261, wherein the metastatic cancer comprises metastasis of bone cancer to the lung.

271. The method of claim 261, wherein the metastatic cancer comprises metastasis of colorectal cancer to the peritoneum, the pancreas, the stomach, the lung, the liver, the kidney, and/or the spleen.

272. The method of claim 261, wherein the metastatic cancer comprises metastasis of stomach cancer to the mesentery, the spleen, the pancreas, the lung, the liver, the adrenal gland, and/or the ovary.

273. The method of claim 261, wherein the metastatic cancer comprises metastasis of leukemia to the lymph nodes, the lung, the liver, the hind limb, the brain, the kidney, and/or the spleen.

274. The method of claim 261, wherein the metastatic cancer comprises metastasis of liver cancer to the intestine, the spleen, the pancreas, the stomach, the lung, and/or the kidney.

275. The method of claim 261, wherein the metastatic cancer comprises metastasis of lymphoma to the kidney, the ovary, the liver, the bladder, and/or the spleen.

276. The method of claim 261, wherein the metastatic cancer comprises metastasis of hematopoietic cancer to the intestine, the lung, the liver, the spleen, the kidney, and/or the stomach.

277. The method of claim 261, wherein the metastatic cancer comprises metastasis of melanoma to lymph nodes and/or the lung.

278. The method of claim 261, wherein the metastatic cancer comprises metastasis of pancreatic cancer to the mesentery, the ovary, the kidney, the spleen, the lymph nodes, the stomach, and/or the liver.

279. The method of claim 261, wherein the metastatic cancer comprises metastasis of prostate cancer to the lung, the pancreas, the kidney, the spleen, the intestine, the liver, the bone, and/or the lymph nodes.

280. The method of claim 261, wherein the metastatic cancer comprises metastasis of ovarian cancer to the diaphragm, the liver, the intestine, the stomach, the lung, the pancreas, the spleen, the kidney, the lymph nodes, and/or the uterus.

281. The method of claim 261, wherein the metastatic cancer comprises metastasis of myeloma to the bone.

282. The method of claim 261, wherein the metastatic cancer comprises metastasis of lung cancer to the bone, the brain, the lymph nodes, the liver, the ovary, and/or the intestine.

283. The method of claim 261, wherein the metastatic cancer comprises metastasis of kidney cancer to the liver, the lung, the pancreas, the stomach, the brain, and/or the spleen.

284. The method of claim 261, wherein the metastatic cancer comprises metastasis of bladder cancer to the bone, the liver and/or the lung.

285. The method of claim 261, wherein the metastatic cancer comprises metastasis of thyroid cancer to the bone, the liver and/or the lung.

286. The method of claim 259 or 260, wherein the cancer is a primary solid tumor.

287. The method of claim 286, wherein the cancer is selected from the group consisting of breast cancer, bladder cancer, lung cancer, brain cancer, ovarian cancer, pancreatic cancer, colorectal cancer, prostate cancer, liver cancer, hepatocellular carcinoma, kidney cancer, stomach cancer, skin cancer, fibroid cancer, lymphoma, virus-induced cancer, oropharyngeal cancer, testicular cancer, thymus cancer, thyroid cancer, melanoma, and bone cancer.

288. The method of claim 287, wherein the cancer is bladder cancer.

289. The method of claim 287, wherein the cancer is lung cancer.

290. The method of claim 287, wherein the cancer is brain cancer.

291. The method of claim 290, wherein the brain cancer is astrocytoma, anaplastic astrocytoma, glioblastoma multiforme, oligodendroglioma, ependymoma, meningioma, schwannoma, or medulloblastoma.

292. The method of claim 291, wherein the brain cancer is astrocytoma.

293. The method of claim 291, wherein the brain cancer is anaplastic astrocytoma.

294. The method of claim 291, wherein the brain cancer is glioblastoma multiforme.

295. The method of claim 291, wherein the brain cancer is oligodendroglioma.

296. The method of claim 291, wherein the brain cancer is ependymoma.

297. The method of claim 291, wherein the brain cancer is meningioma.

298. The method of claim 291, wherein the brain cancer is schwannoma.

299. The method of claim 291, wherein the brain cancer is medulloblastoma.

300. The method of claim 287, wherein the cancer is ovarian cancer.

301. The method of claim 287, wherein the cancer is pancreatic cancer.

302. The method of claim 287, wherein the cancer is colorectal cancer.

303. The method of claim 287, wherein the cancer is prostate cancer.

304. The method of claim 287, wherein the cancer is liver cancer.

305. The method of claim 287, wherein the cancer is hepatocellular carcinoma.

306. The method of claim 287, wherein the cancer is kidney cancer.

307. The method of claim 287, wherein the cancer is stomach cancer.

308. The method of claim 287, wherein the cancer is skin cancer.

309. The method of claim 287, wherein the cancer is fibroid cancer.

310. The method of claim 309, wherein the fibroid cancer is leiomyosarcoma.

311. The method of claim 287, wherein the cancer is lymphoma.

312. The method of claim 311, wherein the lymphoma is Hodgkin's lymphoma, non-Hodgkin's lymphoma, diffuse large B-cell lymphoma, B-cell immunoblastic lymphoma, Natural Killer cell lymphoma, T-cell lymphoma, Burkitt lymphoma or Kaposi's Sarcoma.

313. The method of claim 312, wherein the lymphoma is Hodgkin's lymphoma.

314. The method of claim 312, wherein the lymphoma is non-Hodgkin's lymphoma.

315. The method of claim 312, wherein the lymphoma is diffuse large B-cell lymphoma.

316. The method of claim 312, wherein the lymphoma is B-cell immunoblastic lymphoma.

317. The method of claim 312, wherein the lymphoma is Natural Killer cell lymphoma.

318. The method of claim 312, wherein the lymphoma is T-cell lymphoma.

319. The method of claim 312, wherein the lymphoma is Burkitt lymphoma.

320. The method of claim 312, wherein the lymphoma is Kaposi's Sarcoma.

321. The method of claim 287, wherein the cancer is virus-induced cancer.

322. The method of claim 287, wherein the cancer is oropharyngeal cancer.

323. The method of claim 287, wherein the cancer is testicular cancer.

324. The method of claim 287, wherein the cancer is thymus cancer.

325. The method of claim 287, wherein the cancer is thyroid cancer.

326. The method of claim 287, wherein the cancer is melanoma.

327. The method of claim 287, wherein the cancer is bone cancer.

328. The method of claim 287, wherein the cancer is breast cancer.

329. The method of claim 328, wherein the breast cancer comprises ductal carcinoma, lobular carcinoma, medullary carcinoma, colloid carcinoma, tubular carcinoma, or inflammatory breast cancer.

330. The method of claim 329, wherein the breast cancer comprises ductal carcinoma.

331. The method of claim 329, wherein the breast cancer comprises lobular carcinoma.

332. The method of claim 329, wherein the breast cancer comprises medullary carcinoma.

333. The method of claim 329, wherein the breast cancer comprises colloid carcinoma.

334. The method of claim 329, wherein the breast cancer comprises tubular carcinoma.

335. The method of claim 329, wherein the breast cancer comprises inflammatory breast cancer.

336. The method of claim 259 or 260, wherein the cancer is liquid tumor.

337. The method of claim 336, wherein the liquid tumor is selected from the group consisting of acute myeloid leukemia (AML), acute lymphoblastic leukemia, acute lymphocytic leukemia, acute promyelocytic leukemia, chronic myeloid leukemia, hairy cell leukemia, myeloproliferative disorders, Natural Killer cell leukemia, blastic plasmacytoid dendritic cell neoplasm, chronic myelogenous leukemia (CML), mastocytosis, chronic lymphocytic leukemia (CLL), multiple myeloma (MM), and myelodysplastic syndrome (MDS).

338. The method of claim 337, wherein the liquid tumor is acute myeloid leukemia (AML).

339. The method of claim 337, wherein the liquid tumor is acute lymphoblastic leukemia.

340. The method of claim 337, wherein the liquid tumor is acute lymphocytic leukemia.

341. The method of claim 337, wherein the liquid tumor is acute promyelocytic leukemia.

342. The method of claim 337, wherein the liquid tumor is chronic myeloid leukemia.

343. The method of claim 337, wherein the liquid tumor is hairy cell leukemia.

344. The method of claim 337, wherein the liquid tumor is a myeloproliferative disorder.

345. The method of claim 337, wherein the liquid tumor is Natural Killer cell leukemia.

346. The method of claim 337, wherein the liquid tumor is blastic plasmacytoid dendritic cell neoplasm.

347. The method of claim 337, wherein the liquid tumor is chronic myelogenous leukemia (CML).

348. The method of claim 337, wherein the liquid tumor is mastocytosis.

349. The method of claim 337, wherein the liquid tumor is chronic lymphocytic leukemia (CLL).

350. The method of claim 337, wherein the liquid tumor is multiple myeloma (MM).

351. The method of claim 337, wherein the liquid tumor is myelodysplastic syndrome (MDS).

352. The method of claim 258, wherein the cancer is a pediatric cancer.

353. The method of claim 352, wherein the pediatric cancer is neuroblastoma, Wilms tumor, rhabdomyosarcoma, retinoblastoma, osteosarcoma or Ewing sarcoma.

354. The method of claim 353, wherein the pediatric cancer is neuroblastoma.

355. The method of claim 353, wherein the pediatric cancer is Wilms tumor.

356. The method of claim 353, wherein the pediatric cancer is rhabdomyosarcoma.

357. The method of claim 353, wherein the pediatric cancer is retinoblastoma.

358. The method of claim 353, wherein the pediatric cancer is osteosarcoma.

359. The method of claim 353, wherein the pediatric cancer is Ewing sarcoma.
